(19) United States
(12) Patent Application Publication
(10) Pub. No.: US 2003/0224364 A1
(43) Pub. Date: Dec. 4, 2003

(54) Compositions and methods for identifying and distinguishing orthosomycin biosynthetic loci

(76) Inventors: Chris M. Farnet, Outremont (CA); Emmanuel Zazopoulos, Montreal (CA); Alfredo Staffa, Saint-Laurent (CA)

Correspondence Address: George A. Loud, LORUSSO & LOUD, 3137 Mount Vernon Avenue, Alexandria, VA, 22305, US

(21) Appl. No.: 10/107,431

(22) Filed: Mar. 28, 2002

Related U.S. Application Data
(63) Continuation-in-part of application No. 09/769,734, filed on Jan. 26, 2001.
(60) Provisional application No. 60/279,095, filed on Mar. 28, 2001.
(60) Provisional application No. 60/279,709, filed on Mar. 30, 2001.
(60) Provisional application No. 60/285,214, filed on Apr. 20, 2001.

(51) Int. Cl. C12Q 1/68; G06F 19/00; G01N 33/48; G01N 33/50

(52) U.S. Cl. 435/6; 702/20

(57) ABSTRACT

The invention provides compositions and methods useful to identify orthsomycin biosynthetic gene clusters. The invention also provides compositions and methods useful to distinguish everninomicin-type orthsomycin gene clusters and avilamycin-type orthosomycin gene clusters. An orthosomycin gene cluster may be identified using compositions of the invention such as hybridization probes, PCR primers derived from specific protein families responsible for the unique structural features that distinguish orthosomycins, everninomycin-type orthsosomycins and avilamycin-type orthosomycins. An orthosomycin gene cluster may be identified using compositions of the invention such as the sequence code for the reference sequences stored on computer readable medium.

### CROSS-REFERENCE TO RELATED APPLICATIONS

This application claims benefit under 35 USC §119 of provisional applications U.S. Ser. No. 60/279,095 filed on Mar. 28, 2001, U.S. Ser. No. 60/279,709 filed on Mar. 30, 2001 and U.S. Ser. No. 60/285,214 filed on Apr. 20, 2001 which are hereby incorporated by reference in their entirety for all purposes. This application is a continuation is part of U.S. Ser. No. 09/769,734 filed on Jan. 26, 2001 and claims benefit under 35 USC §120 to U.S. Ser. No. 09/769,734 which is hereby incorporated by reference in its entirety for all purposes.

### FIELD OF INVENTION

The present invention relates to the field of microbiology, and more specifically to genes and organisms involved in the production of orthosomycins.

### BACKGROUND

Orthosomycins are oligosaccharide molecules containing two orthoester saccharide linkages. The general structure of orthosomycins is illustrated below. The saccharide residues in the above orthosomycin are labeled A-H and the key features of orthosomycins, the orthoester linkages are indicated below.

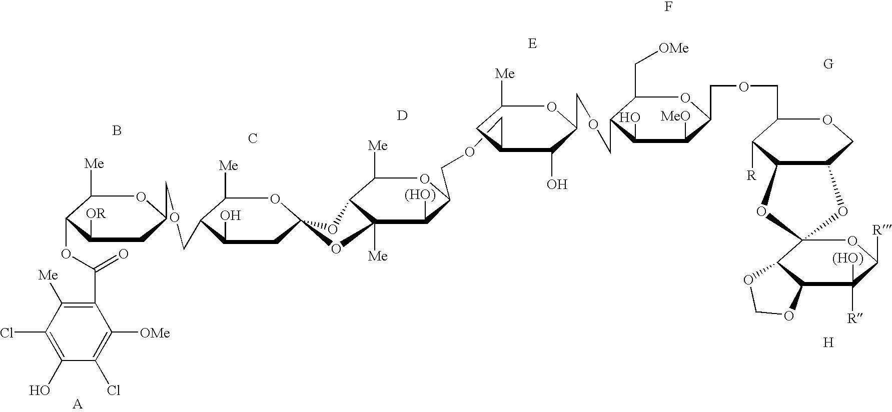

Known orthosomycin compounds can broadly be classified into two classes: (1) the everninomicins that contain an amino- or nitrosugar residue in the terminal position of the oligosaccharide chain, i.e. wherein R is evernitrose in the above molecule; and (2) the avilamycins, curamycins and flambamycins that do not contain an amino- or nitrosugar residue in the terminal position, i.e. wherein R is hydrogen in the above molecule. Within the second class of orthosomycins, the avilamycins and the curamycins differ only in the nature of the acyl side chain found in ester linkage to the C45-hydroxyl group of sugar residue G. Neither the avilamycins nor the curamycins carry a simple methyl group on this hydroxyl. In the everninomicin class, the hydroxyl is generally O-methylated. Flambamycins differ from the avilamycins only at position C23 of sugar residue D, which is a methylene carbon in the avilamycins but carries a hydroxyl group on the flambamycins. The everninomicins may or may not carry a hydroxyl at this position.

Many known orthosomycins have antibiotic activity. There is an urgent need for new anti-microbial agents given the emergence of bacteria resistant to conventional antibiotics. The oligosaccharide class of antibiotics has demonstrated a wide spectrum of antibacterial activity against gram-positive organisms, including methicillin-resistant Staphylococcus aureus, vancomycin-resistant enterococci, and penicillin-resistant pneumococci. It is therefore desirable to develop a means to identify new orthosomycin natural products. Orthosomycin-producing microbes represent an important source of new antibiotics. Accordingly, it is also desirable to develop a means to identify orthosomycin-producing organisms and to distinguish between the classes of orthomycins produced by such orgamisms.

Existing screening methods for identifying orthosomycin-producing microbes are laborious, time-consuming and have not provided sufficient discrimination to date to detect organisms producing orthosomycin natural products at low levels. There is a need for improved tools to detect orthosomycin-producing organisms. There is also a need for tools capable of detecting organisms that produce orthosomycins at levels that are not detected by traditional culture tests. There is also a need for tools that discriminate between the classes of orthosomycin molecules such as avilamycin and everninomicin classes of orthosomycins.

### SUMMARY OF THE INVENTION

The invention provides compositions and methods useful to identify orthsomycin biosynthetic genes. The invention also provides compositions and methods useful to distinguish everninomicin-type orthsomycin gene clusters and avilamycin-type orthosomycin gene clusters. Once target orthosomycin genes are identified, a full length or partial biosynthetic locus for the orthosomycin compound may be isolated according standard methods.

In one aspect of the invention, an orthosomycin gene cluster is identified using compositions of the invention such as hybridization probes or PCR primers. Hybridization probes or PCR primers according to the invention are derived from protein families responsible for the unique structural features that distinguish orthosomydns, everninomycin-type orthsosomycins and avilamycin-type orthosomycins. To identify orthosomycin gene clusters, the hybridization probes or PCR primers are derived from the nucleic acid sequences corresponding to the seventeen protein families GFTE, GFTG, GTFH, HOXG, MTFD, MTFE, MTFF, MTLA, MTIA, OXRV, OXRW, OXRW, PHOD, UNAJ, UEVA, UEVB and UNKU. To identify everninomicin-type orthosomydn gene clusters, the hybridization probes or PCR primers are derived from the nucleic acid sequences corresponding to the nine protein families DACT, DEPF, EPIM, GTFA, MTFG, MTFV, OXBN, OXCO and UNBB. To identify avilamycin-type orthosomycin gene clusters, the hybridization probes or PCR primers are derived from the nucleic acid sequences corresponding to six protein families ABCD, DEPN, MEMD, REBU, UNAI and UNBR.

The invention provides compositions for use in identifying orthosomycin biosynthetic genes, orthosomycin gene fragments, orthosomycin gene clusters or orthosomycin-producing organisms. In one aspect, the invention provides an isolated, purified, or enriched nucleic acid comprising one of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, the sequences complementary thereto, or a fragment comprising at least 10, 15, 20, 25, 30, 35, 40, 50, 76, 100, 150, 200, 300, 400 or 500 consecutive bases of one of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 or the sequences complementary thereto. In another aspect the invention provides the above nucleic acids for use in identifying orthosomycin biosynthetic genes, orthosomycin gene fragments, orthosomycin gene clusters or orthosomycin-producing organisms. The isolated, purified or enriched nucleic acids may comprise DNA, including cDNA, genomic DNA, and synthetic DNA. The DNA may be double stranded or single stranded, and If single stranded may be the coding or non-coding (anti-sense) strand. Alternatively, the isolated, purified or enriched nucleic acids may comprise RNA.

The isolated, purified or enriched nucleic acids of one of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 may be used to prepare one of the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 or fragments comprising at least 5, 10, 15, 20, 25, 30, 35, 40, 50, 75, 100 or 100 consecutive amino acids of one of the polypeptides of SEQ ID NO: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207.

Accordingly, present invention also provides an isolated, purified or enriched nucleic acid which encodes one or the polypeptides of SEQ ID NOS; 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 or fragments comprising at least 5, 10, 15, 20, 25, 30, 35, 40, 50, 75, 100 or 150 consecutive amino acids of one of the polypeptides of SEQ ID NOS: 51, 53, 56, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207. In another aspect, the invention provides the above nucleic adds for use in detecting orthosomycin biosynthetic genes, orthosomycin gene fragments, orthosomycin gene clusters, or orthosomycin producing organisms.

The coding sequences of these nucleic acids may be identical to one of the coding sequences of one of the nucleic acids of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 158, 158, 160, 162, 164, 166, 168, 170, 172, 174, 176, 178, 180, 194, 196, 198, 200, 202, 204, 206, 208 or a fragment thereof or may be different coding sequences which encode one of the polypeptides of SEQ ID NOS: 51, 53, 55, 57, 69, 61, 63, 65, 87, 69, 71, 73, 99, 101, 103, 105, 107, 109, 111, 113, 115, 123, 125, 127, 129, 131, 153, 155, 167, 159, 161, 163, 165, 167, 169, 171, 173, 175, 177, 179, 193, 195, 197, 199, 201, 203, 205, 207, or fragments comprising at least 5, 10, 15, 20, 26, 30, 35, 40, 50, 75, 100 or 150 consecutive amino acids of one of the polypeptides of SEQ ID NOS: 51, 53, 55, 57, 59, 81, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 as a result of the redundancy or degeneracy of the genetic code, for use in detecting orthosomycin biosynthetic genes or orthosomycin producing organisms.

The invention provides compositions for use in identifying everninomicin-type orthosomycin blosynthetic genes, everninomicin-type orthosomycin gene fragments, everninomicin-type orthosomycin gene clusters, and everninomicin and orthosomycin-producing organisms. In one aspect, the invention provides an isolated, purified, or enriched nucleic acid comprising one of the sequences of SEQ ID NOS: 210, 212, 214, 216, 218, 220, 222, 224, 226, 228, 230, 232, 234, 236, 238, 240, 242, 244, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 210, 212, 214, 216, 218, 220, 222, 224, 226, 228, 230, 232, 234, 236, 288, 240, 242, 244 or the sequences complementary thereto. In another aspect, the invention provides the above nucleic acids for use in identifying everninomicin-type orthosomycin genes, everninomicin-type orthosomycin gene fragments, everninomicin-type orthosomycin gene dusters and everninomicin-like orthosomycin producing organisms. The isolated, purified or enriched nucleic acids may comprise DNA, including cDNA, genomic DNA, and synthetic DNA. The DNA may be double stranded or single stranded, and if single stranded may be the coding or non-coding (anti-sense) strand. Alternatively, the isolated, purified or enriched nucleic acids may comprise RNA.

The isolated, purified or enriched nucleic acids of one of SEQ ID NOS: 210, 212, 214, 216, 218, 220, 222, 224, 226, 228, 230, 232, 234, 236, 238, 240, 242, 244 may be used to prepare one of the polypeptides of SEQ ID NOS: 209, 211, 213, 215, 217, 219, 221, 223, 225, 227, 229, 231, 233, 235, 237, 239, 241, 243 or fragments comprising at least 5, 10, 15, 20, 25, 30, 35, 40, 50, 75, 100 or 100 consecutive amino acids of one of the polypeptides of SEQ ID NO: 209, 211, 213, 215, 217, 219, 221, 223, 225, 227, 229, 231, 233, 235, 237, 239, 241, 243.

Accordingly, the present invention also provides an isolated, purified or enriched nucleic acid which encodes one or the polypeptides of SEQ ID NOS: 209, 211, 213, 215, 217, 219, 221, 223, 225, 227, 229, 231, 233, 235, 237, 239, 241, 243 or fragments comprising at least 5, 10, 15, 20, 25, 30, 35, 40, 50, 75, 100 or 150 consecutive amino adds of one of the polypeptides of SEQ ID NOS: 209, 211, 213, 215, 217, 219, 221, 223, 225, 227, 229, 231, 233, 235, 237, 239, 241, 243. In another aspect, the invention provides the above nucleic acids for use in identifying everninomicin-type orthosomycin genes, everninomicin-type orthosomycin gene fragments, everninomicin-type orthosomycin gene clusters, and everninomicin-type orthosomycin producing organisms. The coding sequences of these nucleic acids may be identical to one of the coding sequences of one of the nucleic acids of SEQ ID NOS: 210, 212, 214, 216, 218, 220, 222, 224, 226, 228, 230, 232, 234, 236, 238, 240, 242, 244 or a fragment thereof or may be different coding sequences which encode one of the polypeptides of SEQ ID NOS: 209, 211, 213, 215, 217, 219, 221, 223, 225, 227, 229, 231, 233, 235, 237, 239, 241, 243 or fragments comprising at least 5, 10, 15, 20, 25, 30, 35, 40, 50, 75, 100 or 150 consecutive amino acids of one of the polypeptides of SEQ ID NOS: 209, 211, 213, 215, 217, 219, 221, 223, 225, 227, 229, 231, 233, 235, 237, 239, 241, 243 as a result of the redundancy or degeneracy of the genetic code.

The invention provides compositions for use in identifying avilamycin-type biosynthetic genes avilamycin type orthosomycin gene fragments, avilamycin-type orthosomycin gene clusters, and avilamycin-type orthosomycin producing organisms. In one aspect, the invention provides an isolated, purified, or enriched nucleic acid comprising one of the sequences of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; the sequences complementary thereto; or a fragment comprising at least 10, 15, 20, 25, 30, 35, 40, 50, 75, 100, 150, 200, 300, 400 or 500 consecutive bases of one of the sequences of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; or the sequences complementary thereto. In another aspect, the invention provides the above nucleic acids for use in identifying avilamycin-type orthosomycin genes and avilamycin-type orthosomycin producing organisms. The isolated, purified or enriched nucleic acids may comprise DNA, including cDNA, genomic DNA, and synthetic DNA. The DNA may be double stranded or single stranded, and if single stranded may be the coding or non-coding (anti-sense) strand Alternatively, the isolated, purified or enriched nucleic acids may comprise RNA.

The isolated, purified or enriched nucleic acids of one of SEQ ID NOS: 246, 248, 250, 252, 254, 256 may be used to prepare one of the polypeptides of SEQ ID NOS: 245, 247, 249, 251, 253 and 255 or fragments comprising at least 5, 10, 15, 20, 25, 30, 35, 40, 50, 75, 100 or 100 consecutive amino acids of one of the polypeptides of SEQ ID NO: 245, 247, 249, 251, 253.

Accordingly, the present invention also provides an isolated, purified or enriched nucleic acid which encodes one or the polypeptides of SEQ ID NOS: 245, 2 47, 249, 251, 253 or Genbank accession nos: AAG32068, AAK83183, AAG32069, AAK83172, AAK83171 and AAK83175 or fragments comprising at least 5, 10, 15, 20, 25, 30, 35, 40, 50, 75, 100 or 150 consecutive amino acids of one of the polypeptides of SEQ ID NOS: 245, 247, 249, 251, 253 or Genbank accession nos: AAG32068, AAK83183, AAG32069, AAK83172, AAK83171 and AAK83175. In another aspect, the invention provides the above nucleic acids for use in identifying avilamycin-type orthosomycin genes, avilamycin-type orthosomycin gene fragments, avilamycin-type orthosomycin gene clusters, and avilamycin-type orthosomycin producing organisms. The coding sequences of these nucleic acids may be identical to one of the coding sequences of one of the nucleic acids of SEQ ID NOS: 246, 248, 250, 252, 254, 256 or a fragment thereof or may be different coding sequences which encode one of the polypeptides of SEQ ID NOS; 246, 247, 249, 251, 253 or Genbank accession nos. AAG32068, AAK83183, AAG32069, AAK83172, AAK83171 and AAK83175, or fragments comprising at least 5, 10, 15, 20, 25, 30, 36, 40, 50, 75, 100 or 150 consecutive amino acids of one of the polypeptides of SEQ ID NOS: 245, 247, 249, 251, 253, or GenBank accession nos: AAG32068, AAK83183, AAG32069, AAK83172, AAK83171 and AAK83175 as a result of the redundancy or degeneracy of the genetic code.

The isolated, purified or enriched nucleic acid which encodes one of the polypeptides of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256 may include, but is not limited to: (1) only the coding sequences of one of SEQ ID NOS: 52, 64, 56, 58, 60, 62, 64, 66, 68, 70, 72, 74, 100, 102, 104, 106, 108, 110, 112, 114, 118, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256; (2) the coding sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248; 250, 252, 254, 256 and additional coding sequences, such as leader sequences or proprotein; or (3) the coding sequences of SEQ ID NOS: and non-coding sequences, such as introns or non-coding sequences 5′ and/or 3′ of the coding sequence. Thus, as used herein, the term “polynucleotide encoding a polypeptide” encompasses a polynucleotide which includes only coding sequence for the polypeptide as well as a polynucleotide which includes additional coding and/or non-coding sequence.

The invention relates to polynucleotides which have polynucleotide changes that are “silent”, for example changes which do not alter the amino acid sequence encoded by the polynucleotides of SEQ ID NOS: 62, 64, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 116, 178, 180, 194, 196, 198, 200, 202, 204, 206, 208, 210,.212, 214, 216, 218, 220, 222, 224, 226, 228, 230, 232, 234, 236, 238, 240, 242, 244, 246, 248, 250, 252, 254, 256, for use in detecting orthosomycin biosynthetic genes and orthosomycin-producing organisms. The invention also relates to polynucleotides which have nucleotide changes which result in amino acid substitutions, additions, deletions, fusions and truncations of the polypeptides of SEQ ID NOS: 51, 63, 55, 57, 59, 61, 63, 65, 67, 69, 71, 73, 99, 101, 103, 106, 107, 109, 111, 113, 115, 123, 125, 127, 129, 131, 153, 155, 157, 159, 161, 163, 165, 167, 169, 171, 173, 175, 177, 179, 193, 145, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255 for use in identifying orthosomycin biosynthetic genes and orthosomycin producing organisms.

In one aspect the compositions of the invention are used as probes to identify samples harbouring orthosomycin biosynthetic genes and orthosomycin biosynthetic loci. Samples may be in the form of environmental biomass, pure or mixed microbial culture, isolated genomic DNA from pure or mixed microbial culture, genomic DNA libraries from pure or mixed microbial culture. The compositions are used in polymerase chain reaction, and nucleic acid hybridization techniques well known to those skilled in the art.

In another embodiment, environmental samples that harbour microorganisms with the potential to produce orthosomycins are identified by PCR methods. Nucleic acids contained within the environmental sample are contacted with primers derived from the invention so as to amplify target orthosomycin biosynthetic gene sequences. Environmental samples deemed to be positive by PCR are then pursued to identify and isolate the orthosomycin gene cluster and the microorganism that contains the target gene sequences. The orthosomycin gene cluster may be identified by generating genomic DNA libraries (for example, cosmid, BAC, etc.) representative of genomic DNA from the population of various microorganisms contained within the environmental sample, locating genomic DNA clones that contain the target sequences and possibly overlapping clones (for example, by hybridization techniques or PCR), determining the sequence of the desired genomic DNA clones and deducing the ORFs of the orthosomycin biosynthetic locus. The microorganism that contains the orthosomycin biosynthetic locus may be identified and isolated, for example, by colony hybridization using nucleic acid probes derived from either the invention or the newly identified orthosomycin biosynthetic locus. The isolated orthosomycin biosynthetic locus may be introduced into an appropriate surrogate host to achieve heterologous production of the orthosomycin compound(s); alternatively, if the microorganism containing the orthosomycin biosynthetic locus is identified and isolated it may be subjected to fermentation to produce the orthosomycin compound(s).

In another embodiment of the invention, a microorganism that harbours an orthosomycin gene cluster is first identified and isolated as a pure culture, for example, by colony hybridization using nucleic acid probes derived from the invention. Beginning with a pure culture, a genomic DNA library (for example, cosmid, BAC, etc.) representative of genomic DNA from this single species is prepared, genomic DNA clones that contain the target sequences and possibly overlapping clones are located using probes derived from the invention (for example, by hybridization techniques or PCR), the sequence of the desired genomic DNA clones is determined and the ORFs of the orthosomycin biosynthetic locus are deduced. The microorganism containing the orthosomycin biosynthetic locus may be subjected to fermentation to produce the orthosomycin compound(s) or the orthosomycin biosynthetic locus may be introduced into an appropriate surrogate host to achieve heterologous production of the orthosomycin compound(s).

In another aspect of the invention, an orthosomycin gene cluster is identified in silico using one or more sequences selected from orthosomycin-specific nucleic acid code, everninomicin-specific nucleic acid code, avilamycin-specific nucleic acid code, orthosomycin-specific polypeptide code, everninomicin-specific polypeptide code and avilamycin-specific polypeptide code as taught by the invention. A query from a set of query sequences stored on computer readable medium is read and compared to a subject selected from the reference sequences of the invention. The level of similarity between said subject and query is determined and queries sequences representing orthosomycin genes are identified.

It is understood that the invention, having provided, compositions and methods to identify othosomycin biosynthetic gene cluster, everninomycin-type biosynthetic gene clusters and avilamycin-type biosynthetic gene clusters, further provides orthosomycins, everninomicin-type orthosomycins, and avilamycin-type orthsomycins produced by the biosynthetic gene dusters identified.

### BRIEF DESCRIPTION OF THE DRAWINGS

FIG. 1 is a block diagram of a computer system which implements and executes software tools for the purpose of comparing a query to a subject, wherein the subject is selected from the reference sequences of the invention

FIGS. 2A, 2B, 2C and 2D are flow diagrams of a sequence comparison software that can be employed for the purpose of comparing a query to a subject, wherein the subject is selected from the reference sequences of the invention, wherein

FIG. 2A is the query initialization subprocess of the sequence comparison software,

FIG. 2B is the subject datasource initilization subprocess of the sequence comparison software,

FIG. 2C illustrates the comparison subprocess and the analysis subprocess of the sequence comparison software,

FIG. 2D is the Display/Report subprocess of the sequence comparison software.

FIG. 3 is a flow diagram of the comparator algorithm (238) of FIG. 2C which is one embodiment of a comparator algorithm that can be used for pairwise determination of similarity between a query/subject pair.

FIG. 4 is a flow diagram of the analyzer algorithm (244) of FIG. 2C which is one embodiment of an analyzer algorithm that can be used to assign identity to a query sequence, based on similarity to a subject sequence, where the subject sequence is a reference sequence of the invention.

FIG. 5 is a schematic representation comparing the an avilamycin-type biosynthetic locus from Streptomyces mobaraensis (AVIA) to the avilamycin A biosynthetic locus from Streptomyces viridochromogenes Tu57 (AVIL), ORFs in the loci are identified by a four-letter protein family designation.

FIG. 6 illustrates a biosynthetic scheme wherein members of the proteins families commonly found in orthosomycin biosynthetic loci, namely KASA (EVEA ORF 17, SEQ ID NO: 84; EVER ORF 14, SEQ ID NO: 83; AVIA ORF 13, SEQ ID NO: 81; and AVIL ORF 15, Genbank accession no: AAK83178), PKSO (EVEA ORF 16, SEQ ID NO: 185; EVER ORF 32, SEQ ID NO: 183; AVIA ORF 14, SEQ ID NO: 181; and AVIL ORF 16, Genbank accession no: AAK83194), MTFA (EVEA ORF 44, SEQ ID NO: 97; EVER ORF 11, SEQ ID NO: 95; AVIA ORF 38, SEQ ID NO: 93), and HOMX (EVEA ORF 20, SEQ ID NO: 79; EVER ORF 20, SEQ ID NO: 77; AVIA ORF 36, SEQ ID NO: 75) provide for the formation of the dichloroisoeverninic moiety found in the ester linkage to the sugar residue B of orthosomycin oligosaccharides.

FIG. 7 illustrates two alternative biosynthetic routes wherein members of protein families diagnostic of orthosomycin biosynthetic loci, namely OXRW (AVIA ORFs 24 and 33 (SEQ ID NOS: 153 and 159); AVIL GenBank accession no. AAK83187; EVER ORFs 18 and 26 (SEQ ID NOs: 155 and 161); EVEA ORFs 11 and 30 (SEQ ID NO: 157 and 163)), and OXRV (AVIA ORF 19 (SEQ ID NO: 167), EVEA ORF 6 (SEQ ID NO: 173), AVIL GenBank accession no. AAK83181), EVER ORF 31 (SEQ ID NO: 169)) provide for the formation of the orthoester linkages joining residues C and D of orthosomycin oligosaccharides.

FIG. 8 illustrates a biosynthetic scheme wherein members of the proteins families diagnostic of everninomicin-type orthosomycin gene clusters and everninomicin-type orthosomycin producers, including DATC (EVER ORF 43 (SEQ ID NO: 209); EVEA ORF 37 (SEQ ID NO: 211)); MTFV (EVER ORF 44 (SEQ ID NO: 229), EVEA ORF 38 (SEQ ID NO: 231)); EPIM (EVER ORF 45 (SEQ ID NO: 217), EVEA ORF 39 (SEQ ID NO: 219)), DEPF (EVER ORF 46 (SEQ ID NO: 213), EVEA ORF 40 (SEQ ID NO: 215)), and OXBN (EVER ORF 42 (SEQ ID NO: 233), EVEA 36 (SEQ ID NO: 235)) provide for the formation of amino- and nitrosugar residues characterisitc of everninomicin-type orthosomycins.

FIG. 9 is a represents a picture of a 1% agarose gel stained with ethidium bromide generated in the PCR amplification experiments described in Example 8.

FIG. 10 is a schematic representation comparing the everninomicin biosynthetic locus from Micromonospora carbonacae var. aurantiaca (EVER) to the everninomicin biosynthetic locus from Micromonospora carbonacea var. africana (EVEA), ORFs in the loci are identified by a four-letter protein family designation.

### DETAILED DESCRIPTION OF THE INVENTION

The invention provides compositions and methods for identifying orthosomycin gene clusters and orthosomycin producing organisms. The invention also provides compositions and methods for distinguishing between everninomicin-type orthosomycin gene clusters and avilamycin-type orthosomycin gene cluster, and to distinguish between everninomicin-type orthosomycin producers and avilamycin-type orthosomycin producers. To provide the compositions and methods of the invention, the full-length biosynthetic locus for a member of each of the two classes of orthosomycin compounds was identified, sequenced and annotated. The biosynthetic locus for everninomicin in Micromonospora carbonacea var. aurantiaca (EVER) spans approximately 60 kb and contains 49 ORFs encoding proteins involved in the biosynthesis of everninomicin. The biosynthetic locus for an avilamycin-like compound from Streptomyces mobaraensis (AVIA) spans approximately 50 kb and contains 42 ORFs encoding proteins involved in the biosynthesis of an avilamycin-type compound.

Analysis of EVER and AVIA has revealed seventeen (17) protein families responsible for structural features common to all orthosomycin molecules and indicative of an orthosomycin biosynthetic locus. A member of each of these 17 protein families has been found in EVER, namely EVER ORFs 5, 8, 9, 12, 13, 15, 17 to 19, 24 to 26, 31, 33, 35 and 40 (SEQ ID NOS: 113, 65, 201, 71, 125, 101, 195, 155, 107, 53, 205, 161, 169, 177, 59 and 129 respectively), and also in AVIA, namely ORFs 1 to 3, 5, 9, 18, 19, 22 to 26, 31 to 34 and 37 (SEQ ID NOS: 123, 203, 127, 57, 199, 165, 167, 99, 105, 153, 111, 193, 51, 63, 159, 175 and 69 respectively). In EVER two of the protein families are fused together to form ORF 31 (SEQ ID NO: 169). A member of the 17 protein families has also been found in the biosynthetic locus for everninomicin from Micromonospora carbonacea var. africana and the biosynthetic locus for an avilamycin compound from Streptomyces viridochromogenes Tu57. Sequences from these 17 protein families form the basis for compositions and methods for identifying gene clusters involved in the biosynthesis of orthosomycins and for compositions and methods for identifying orthosomycin-producing organisms.

Analysis of EVER and AVIA has revealed nine (9) protein families that distinguish everninomicin-type orthosomycin biosynthetic loci from avilamycin-type orthosomicin biosynthetic loci. A member of each of these nine protein families has been found in EVER, namely EVER ORFs 3, 4, 21, 42, 43, 44, 45, 46 and 47 (SEQ ID NOS: 225, 237, 221, 233, 209, 229, 217, 213 and 241 respectively). A member of each of the 9 protein families has also been found in the biosynthetic locus for everninomicin from Micromonospora carbonacea var. africana. No members of these nine protein families were found in biosynthetic loci for avilamycin-type orthosomycins, including AVIA, the biosynthetic locus for an avilamycin compound from Streptomyces viridochromogenes Tu57. Sequences from these nine protein families form the basis for compositions and methods for identifying gene clusters involved in the biosynthesis of everninomicin-type orthosomycins and for compositions and methods for identifying everninomicin-type orthosomycin producing organisms.

Analysis of EVER and AVIA has revealed six (6) protein families that distinguish avilamycin-type orthosomycin biosynthetic loci from everninomicin-type orthosomycin biosynthetic loci. A member of each of these six protein families has been found in AVIA, namely AVIA ORFs 6, 7, 10, 21, 27 and 28 (SEQ ID NOS: 253, 251, 255, 247, 245 and 249). A member of the 6 protein families has also been found in the biosynthetic locus for an avilamycin compound from Streptomyces viridochromogenes Tu57. No member of these six protein families were found in biosynthetic loci for everninomicin-type orthorsomycins, including EVER and the biosynthetic locus for everninomicin from Micromonospora carbonacea var. africana. Sequences from these six protein families form the basis for compositions and methods for identifying gene clusters involved in the biosynthesis of avilamycin-type orthosomycins and for compositions and methods for identifying avilamycin-type orthosomycin producing organisms.

The compositions and methods of the invention can be used to detect the presence of virtually any organism that contains DNA for the production of orthosomycins (both everninomicin-type orthosomycins and avilamycin-type orthosomycins) regardless of the level at which genes for orthosomycin production are expressed by the organism or the amount of orthosomycin produced by the organism. Detection of nucleic acid sequences or amino acid sequences involved in the production of orthosomycins allows for the detection of new orthosomycin natural products, which natural products may not be produced by the organism under standard laboratory conditions or under the typical environmental conditions in which the organism is found in nature. Detection of the nucleic acid sequences or amino acid sequences involved in the production of orthosomycins allows for the detection of new orthosomycins which are produced at levels too low for detection by culture tests. Detection of nucleic acid sequences or amino acid sequences involved in the production of orthosomycins allows for the detection of new orthosomycin producers (both everninomicin-type orthosomycin producers and avilamycin-type orthosomycin producers) representing a source of new orthosomycin natural products.

Detection of the presence or absence of open reading frames necessary for orthosomycin production can be accomplished by hybridization probes or PCR primers based upon the compositions and teachings of the invention. Screening with a probe can be done either in silico or by traditional hybridization screening techniques.

Throughout the description and the figures, the biosynthetic locus for everninomicin from Micromonospora carbonacae var. aurantiaca NRRL 2997 is sometimes referred to as EVER, the biosynthetic locus for everninomicin from Micromonospora carbonacea var. africana (ATCC 39149, SCC 1413) is sometimes referred to as EVEA, the biosynthetic locus for an avilamycin-like compound from Streptomyces mobarensis is sometimes referred to as AVIA, and the biosynthetic locus for an avilamycin compound from Streptomyces viridochromogenes Tu57 is sometimes referred to as AVIL.

The ORFs in EVER, EVEA, AVIA and AVIL are assigned a putative function and grouped together in families based on homology to known proteins, or lack of homology to any known proteins. To correlate structure and function, the protein families are given a four-letter designation used throughout the description and figures as indicated on Table I.

Family descriptions

| Families | Proposed function |
|---|---|
| ABCD | ABC transporter; ATP-binding cassette transmembrane transporter; includes |
| | proteins with similarity to Mdr proteins of mammalian tumor cells that confer |
| | resistance structurally unrelated chemotherapeutic agents; resistance determinant |
| DATC | dehydratase/aminotransferase; SMAT family (secondary metabolism |
| | aminotransferase); transaminase |
| DEPA | dehydratase/epimerase; dTDP-glucose 4,6-dehydratases, catalyze the second step |
| | in 6-deoxyhexose biosynthesis; includes AveBII, StrE, OleE, DesIV, UrdH, SpcJ |
| DEPD | dehydratase/epimerase; may be specific for dGDP-mannose; similar to DEPE |
| DEPE | dehydratase/epimerase, NAD-dependent; includes enzymes that may be specific |
| | for dGDP-mannose; similar to DEPD |
| DEPF | dehydratase/epimerase; includes NDP-hexose 4-ketoreductase TyICIV, AveBIV, |
| | EryBIV, MegDV |
| DEPG | dehydratase/epimerase; includes the dTDP-ketodeoxyhexose reductases SnoG, |
| | LanZ3, DnmV, AknM |
| DEPH | dehydratase/epimerase/ketoreductase; similar to glucose 4-epimerases involved in |
| | primary metabolism |
| DEPI | dehydratase/epimerase, NAD-dependent; 4-ketoreductase; most similar to NDP- |
| | glucose 4-epimerases; includes Spcl |
| DEPJ | dehydratase/epimerase/ketoreductase; most similar to UDP-glucose-4-epimerases |
| DEPN | dehydratase/epimerase; similar to many plant putative dTDP-glucose-4,6- |
| | dehydratases |
| DHYA | dehydratase, deoxysugar; 2,3-dehydratases; 2 similar (repeated) substrate or |
| | cofactor binding motifs in this class; includes EryBVI, DnmT, LanS |
| EFFA | efflux; transmembrane transporter |
| ENGA | endoglucanase; hydrolysis of 1,4-beta-D-glucosidic linkage; likely resistance |
| | determinant; believed to be a secreted protein |
| EPIM | epimerase; NDP-hexose epimerase; TDP-4-ketohexose-3,5-epimerases, convert |
| | TDP-4-keto-6-deoxy-D-glucose to TDP-4-keto-6-deoxy-L-mannose (TDP-4-keto-L- |
| | rhamnose); includes EryBVII, DnmU, AknL, OleL, LanZ1 |
| GTFA | glycosyl transferase; includes EryBV, EryCIII, DesVII, TyIMII, Dau/DnrH, MtmGI-IV, |
| | LanGT1-4 |
| GTFE | glycosyltransferase; no homology to other GTFs |
| GTFG | glycosyltransferase; no homology to other GTFs |
| GTFH | glycosyltransferase; no homology to other GTFs |
| HOXG | oxidase domain homology |
| HOXM | hydroxylase/halogenase; strong similarity to non-heme |
| | halogenase/oxygenase/hydroxylases |
| HYDH | haloacid dehalogenase-like hydrolase; similarity to haloalkanoic acid |
| | dehydrogenases, convert 2-halo acids to 2-hydroxy acids plus halide |
| KASA | ketoacyl synthase; homology to 3-oxoacyl-[acyl-carrier-protein synthase III (KAS III, |
| | acetylCoA ACP transacylase), the principle enzyme responsible for the initiation of |
| | branched-chain fatty acid biosynthesis |
| KINB | kinase; similar to glucose kinase |
| MEMD | membrane protein; includes DrrB daunorubicin resistance transmembrane protein; |
| | resistance determinant |
| MEMK | membrane protein; Na/proton antiporter-like |
| MTBA | O-methyltransferase; includes TylF macrocin-O-methyltransferase, spinosyn SpnH |
| | rhamnose O-methyltransferase |
| MTFA | methyltransferase, SAM-dependent; includes O-methyltransferases, N,N- |
| | dimethyltransferases (e.g. spinosyn SpnS N-dimethyltransferase), C- |
| | methyltransferases |
| MTFD | methyltransferase |
| MTFE | methyltransferase |
| MTFF | methyltransferase domain homology |
| MTFG | methyltransferase domain homomogy |
| MTFH | methyltransferase; includes SnoY, OleY, TylE, SpnI rhamnose O-methyltransferase |
| MTFV | C-methyltransferase, SAM-dependent; includes tylCIII NDP-hexose 3-C- |
| | methyltransferase, NovU C-methyltransferase, EryBIII, DnrX |
| MTIA | resistance methyltransferase; similarity to SpoU family of 23S rRNA |
| | methyltransferases |
| MTLA | rRNA methyltransferase; includes avilamycin AviRa; resistance determinant |
| NUTA | nucleotidyltransferase; dNDP-glucose synthase; alpha-D-glucose-1-phosphate |
| | thymidylyltransferase; catalyze the first step in 6-deoxyhexose biosynthesis |
| OXBN | flavin-dependent oxidoreductase; shows strong homology to eukaryotic acyl CoA |
| | dehydrogenases; includes DnmZ |
| OXCO | oxidoreductase; blue copper oxidoreductase; similar to bilirubin oxidase and B. |
| | subtilis outer spore coat protein involved in brown pigmentation during |
| | sporogenesis; weak similarity to phenoxazinone synthase and yeast laccases |
| OXRA | oxidoreductase; NAD(H)-dependent dehydrogenases; includes OleW putative 3- |
| | ketoreductase, RdmF |
| OXRF | oxidoreductase; includes strT, eryBII, tylCII NDP-hexose 2,3-enoyl reductase |
| OXRT | oxidoreductase; dehydrogenase E1 component beta subunit; strongest homology |
| | to acetoin: DCPIP oxidoreductases from a variety of organisms |
| OXRU | oxidoreductase; dehydrogenase E1 component alpha subunit; strongest homology |
| | to thiamine pyrophosphate-dependent acetoin dehydrogenases from a variety of |
| | organisms |
| OXRV | oxidoreductase; dioxygenase; has domain homology to Pseudomonas putative |
| | alpha-ketoglutarate-dependent hypophosphite dioxygenase, a family of alpha- |
| | ketoglutarate-dependent dioxygenases that catalyze the oxidation of their |
| | respective substrates by using molecular oxygen as the immediate electron |
| | acceptor |
| OXRW | oxidoreductase; dioxygenase; includes SnoN, SnoK; note that |
| | OXRX = UNAJ + OXRW; show homology to Pseudomonas putative alpha- |
| | ketoglutarate-dependent hypophosphite dioxygenase, a family of alpha- |
| | ketoglutarate-dependent dioxygenases that catalyze the oxidation of their |
| | respective substrates by using molecular oxygen as the immediate electron |
| | acceptor; also includes proteins with homology to proline 4-hydroxylase, |
| | mammalian phytanoyl-CoA alpha hydroxylase |
| OXRX | oxidoreductase; dioxygenase; fusion of UNAJ and OXRW (OXRX = UNAJ + OXRW); |
| | the UNAJ portion may contain SAM-binding (methyltransferase) motifs; has domain |
| | homology to Pseudomonas putative alpha-ketoglutarate-dependent hypophosphite |
| | dioxygenase, a family of alpha-ketoglutarate-dependent dioxygenases that catalyze |
| | the oxidation of their respective substrates by using molecular oxygen as the |
| | immediate electron acceptor |
| PHOD | phosphatase; domain homology to phosphoglycolate phosphatase and other |
| | hydrolases |
| PKSO | iterative type I polyketide synthase; includes orsellinic acid synthase AviM |
| REBU | regulator, putative; small proteins that consist mostly of a HTH domain of the LuxR- |
| | type; strong homology to the C-terminal (LuxR-type) HTH domains of AbsA2-type |
| | response regulators |
| REBV | regulator, putative; small proteins that contain a C-terminal HTH domain having |
| | some homology to the C-terminal (LuxR-type) HTH domains of AbsA2-type |
| | response regulators |
| REGL | regulator, similar to Lacl family transcriptional regulators (generally repressors that |
| | lose high-affinity DNA binding in the presence of small molecule effectors) |
| UEVA | contains domain found in MoaA/NifB/PqqA molybdenum cofactor biosynthesis |
| | proteins, coenzyme PQQ synthesis protein and NirJ heme biosynthesis protein |
| UEVB | contains central double-stranded beta helix domain found in a variety of proteins |
| | and proposed to be involved in carbohydrate binding or protein-protein interactions, |
| | depending on the context in which it is found; this domain is found in e.g. mannose- |
| | 1-phosphate guanylyltransferases(GDP), dTDP-4-keto-6-deoxyglucose-3,5- |
| | epimerase (dTDP-L-rhamnose synthase), tetracenomycin TcmJ putative b-ring |
| | cyclase, elloramycin ElmJ, Pseudomonas WbjC putative nulcleotide-binding protein |
| | involved in O-antigen biosynthesis |
| UNAI | unknown; homolog of hypothetical S. coelicolor protein |
| UNAJ | unknown; contains domain found in many bacterial proteins, including hypothetical |
| | proteins and a variety of methyltransferases (a portion of this domain is also found |
| | in eukaryotic RNA helicases); also occurs as a domain fused to an everninomicin |
| | oxidoreductase (OXRX = UNAJ + OXRW) |
| UNBB | unknown |
| UNBR | unknown; N-terminal domain homology to some sugar |
| | dehydratase/epimerase/ketoreductases |
| UNKU | unknown |

“Isolated” means that the material is removed from its original environment, e.g. the natural environment if it is naturally occurring. For example, a naturally-occurring polynucleotide or polypeptide present in a living organism is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The term “purified” does not require absolute purity; rather, it is intended as a relative definition. Individual nucleic acids obtained from a library have been conventionally purified to electrophoretic homogeneity. The sequences obtained from these clones could not be obtained directly from a large insert library, such as a cosmid library, or from total organism DNA. The purified nucleic acids of the present invention have been purified from the remainder of the genomic DNA in the organism by at least 10⁴ to 10⁶ fold. However, the term “purified” also includes nucleic acids which have been purified from the remainder of the genomic DNA or from other sequences in a library or other environment by at least one order of magnitude, preferably two or three orders of magnitude, and more preferably four or five orders of magnitude.

“Recombinant” means that the nucleic acid is adjacent to “backbone” nucleic acid to which it is not adjacent in its natural environment. “Enriched” nucleic acids represent 5% or more of the number of nucleic acid inserts in a population of nucleic acid backbone molecules. “Backbone” molecules include nucleic acids such as expression vectors, self-replicating nucleic acids, viruses, integrating nucleic acids, and other vectors or nucleic acids used to maintain or manipulate a nucleic acid of interest. Preferably, the enriched nucleic acids represent 15% or more, more preferably 50% or more, and most preferably 90% or more, of the number of nucleic acid inserts in the population of recombinant backbone molecules.

“Recombinant” polypeptides or proteins refers to polypeptides or proteins produced by recombinant DNA techniques, i.e. produced from cells transformed by an exogenous DNA construct encoding the desired polypeptide or protein. “Synthetic” polypeptides or proteins are those prepared by chemical synthesis.

The term “gene” means the segment of DNA involved in producing a polypeptide chain; it includes regions preceding and following the coding region (leader and trailer) as well as, where applicable, intervening regions (introns) between individual coding segments (exons).

A DNA “coding sequence” or “nucleotide sequence encoding” a particular polypeptide or protein, is a DNA sequence which is transcribed and translated into a polypeptide or protein when placed under the control of appropriate regulatory sequences

“Oligonucleotide” refers to a nucleic acid, generally of at least 10, preferably 15 and more preferably at least 20 nucleotides, preferably no more than 100 nucleotides, that are hybridizable to a genomic DNA molecule, a cDNA molecule, or an mRNA molecule encoding a gene, mRNA, cDNA or other nucleic acid of interest.

“Orthosomycin producer” or “orthosomycin-producing organism” refers to a microorganism which carries the genetic information necessary to produce an orthosomycin compound, whether or not the organism is known to produce an orthosomycin product. The terms apply equally to organisms in which the genetic information to produce an orthosomycin compound is found in the organism as it exists in its natural environment, and to organisms in which the genetic information is introduced by recombinant techniques. Orthosomycin producers include organisms of the family Micromonosporaceae, of which preferred genera include Micromonospora, Actinoplanes and Dactylosporangium; the family Streptomycetaceae, of which preferred genera include Streptomyces and Kitasatospora; and the family Pseudonocardiaceae, of which preferred genera are Amycolatopsis and Saccharopolyspora.

Deposits:

Three deposits of a E.coli DH10B strain each harboring a cosmid clone which together span the everninomicin biosynthetic locus from Micromonospora carbonacea aurantiaca were made on Jan. 24, 2001 with the International Depositary Authority of Canada (IDAC), 1015 Arlington Street, Winnipeg, Manitoba, Canada R3E 3R2. The deposits were assigned accession nos. IDAC 240101-1, IDAC 240101-2 and IDAC 240101-3. Two deposits of a E.coli DH1 OB strain each harboring a cosmid clone which together span the avilamycin-like biosynthetic locus from Streptomyces mobarensis were made on Feb. 27, 2001 with the International Depositary Authority of Canada (IDAC), 1015 Arlington Street, Winnipeg, Manitoba, Canada R3E 3R2. The deposits were assigned accession nos. IDAC 270201-1 and IDAC 270201-2. The E. coli strain deposits are referred to herein as “the deposited strains”.

The deposited strains together comprise the complete biosynthetic locus for everninomicin from Micromonospora carbonacae var. aurantiaca and the avilamycin-type compound from Streptomyces mobarensis. The sequence of the polynucleotides comprised in the deposited strains, as well as the amino acid sequence of any polypeptide encoded thereby are controlling in the event of any conflict with any description of sequences herein.

The deposits of the deposited strains have been made under the terms of the Budapest Treaty on the International Recognition of the Deposit of Micro-organisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement, such as that required under 35 U.S.C. §112. A license may be required to make, use or sell the deposited strains, and compounds derived therefrom, and no such license is hereby granted.

Structural features common to all orthosomycins require one or more proteins selected from a group of 17 specific protein families, namely GTFE, GTFG, GTFH, HOXG, MTFD, MTFE, MTFF, MTLA, MTIA, OXRV, OXRW, OXRW, PHOD, UNAJ, UEVA, UEVB and UNKU. These 17 protein families include two OXRW families, although in EVER the second OXRW family is designated OXRX as it is a fusion of proteins from the UNAJ and OXRW families. A polypeptide representing a member of any one of these 17 protein families or a polynucleotide encoding a polypeptide representing a member of any one of these 17 protein families is considered diagnostic of an orthosomycin gene cluster and an orthosomycin-producing organism.

It is not expected that an orthosomycin biosynthetic locus will contain a member of each of the 17 protein families considered diagnostic of orthosomycin biosynthetic loci. For example, the UEVB and MTIA protein families are not found in the EVEA locus. Nonetheless, the UEVB and MTIA protein families are considered to be indicative of an orthosomycin locus as they are found in the AVIA, AVIL and EVER loci and no other homologues have been found to date. The presence of at least one, preferably 2, more preferably 4, still more preferably 6, still more preferably 8, still more preferably 10 or more of the seventeen protein families GTFE, GTFG, GTFH, HOXG, MTFD, MTFE, MTFF, MTLA, MTIA, OXRV, OXRW, OXRW, PHOD, UNAJ, UEVA, UEVB and UNKU indicates the presence of an orthosomycin biosynthetic locus and an orthosomycin producing organism.

Members of protein family GTFE include polypeptides selected from AVIA ORF 31 (SEQ ID NO: 51), AVIL GenBank accession no. AAK83192, EVER ORF 24 (SEQ ID NO: 53), EVEA ORF 33 (SEQ ID NO: 55) and polypeptides having at least 99%, at least 95%, at least 90%, at least 85%, at least 80% or at least 70% homology to a polypeptide having the sequence of SEQ ID NOS: 51, 53, 55 or AVIL GenBank accession no. AAK83192 as determined using the BLASTP algorithm with the default parameters.

Members of protein family GTFG include polypeptides selected from AVIA ORF 5 (SEQ ID NO: 57), AVIL GenBank accession no. AAK83170, EVER ORF 35 (SEQ ID NO: 59), EVEA ORF 27 (SEQ ID NO: 61) and polypeptides having at least 99%, at least 95%, at least 90%, at least 85%, at least 80% or at least 70% homology to a polypeptide having the sequence of SEQ ID NOS: 57, 59, 61 or AVIL GenBank accession no. AAK83170 as determined using the BLASTP algorithm with the default parameters.

Members of protein family GTFH include polypeptides selected from AVIA ORF 32 (SEQ ID NO: 63), AVIL GenBank accession no. AAK83193, EVER ORF 8 (SEQ ID NO: 65), EVEA ORF 31 (SEQ ID NO: 67), and polypeptides having at least 99%, at least 95%, at least 90%, at least 85%, at least 80% or at least 70% homology to a polypeptide having the sequence of SEQ ID NOS: 63, 65, 67 or AVIL GenBank accession no. AAK83193 as determined using the BLASTP algorithm with the default parameters.

Members of protein family HOXG include polypeptides selected from AVIA ORF 37 (SEQ ID NO: 69), EVER ORF 12 (SEQ ID NO: 71), EVEA ORF 43 (SEQ ID NO: 73), and polypeptides having at least 99%, at least 95%, at least 90%, at least 85%, at least 80% or at least 70% homology to a polypeptide having the sequence of SEQ ID NOS: 69, 71 or 73 as determined using the BLASTP algorithm with the default parameters.

Members of protein family MTFD include polypeptides selected from AVIA ORF 22 (SEQ ID NO: 99), AVIL GenBank accession no. AAK83184, EVER ORF 15 (SEQ ID NO: 101), EVEA ORF 8 (SEQ ID NO: 103), and polypeptides having at least 99%, at least 95%, at least 90%, at least 85%, at least 80% or at least 70% homology to a polypeptide having the sequence of SEQ ID NOS:99, 101, 103 or AVIL GenBank accession no. AAK83184 as determined using the BLASTP algorithm with the default parameters.

Members of protein family MTFE include polypeptides selected from AVIA ORF 23 (SEQ ID NO: 105), AVIL GenBank accession no. AAK83186, EVER ORF 19 (SEQ ID NO: 107), EVEA ORF 10 (SEQ ID NO: 109), and polypeptides having at least 99%, at least 95%, at least 90%, at least 85%, at least 80% or at least 70% homology to a polypeptide having the sequence of SEQ ID NOS: 105, 107, 109 or AVIL GenBank accession no. AAK83186 as determined using the BLASTP algorithm with the default parameters.

Members of protein family MTFF include polypeptides selected from AVIA ORF 25 (SEQ ID NO: 111), AVIL GenBank accession no. AAK83188, EVER ORF 5 (SEQ ID NO: 113), EVEA ORF 12 (SEQ ID NO: 115) and polypeptides having at least 99%, at least 95%, at least 90%, at least 85%, at least 80% or at least 70% homology to a polypeptide having the sequence of SEQ ID NOS: 111, 113, 115 or AVIL GenBank accession no. AAK83188 as determined using the BLASTP algorithm with the default parameters

Members of protein family MTLA include polypeptides selected from AVIA ORF 3 (SEQ ID NO: 127), AVIL GenBank accession no. AAG32067, EVER ORF 40 (SEQ ID NO: 129), EVEA ORF 45, SEQ ID NO: 131) and polypeptides having at least 99%, at least 95%, at least 90%, at least 85%, at least 80% or at least 70% homology to a polypeptide having the sequence of SEQ ID NOS: 127, 129, 131 or AVIL GenBank accession no. AAG32067 as determined using the BLASTP algorithm with the default parameters.

Members of protein family MTIA include polypeptides selected from AVIA ORF 1 (SEQ ID NO: 123), AVIL GenBank accession no. AAG32066, EVER ORF 13 (SEQ ID NO: 125) and polypeptides having at least 99%, at least 95%, at least 90%, at least 85%, at least 80% or at least 70% homology to a polypeptide having the sequence of SEQ ID NOS: 123, 125 or AVIL GenBank accession no. AAG32066 as determined using the BLASTP algorithm with the default parameters.

Members of protein family OXRV include polypeptides selected from AVIA ORF 24 (SEQ ID NO: 153), AVIL GenBank accession no. AAK83187, EVER ORF 18 (SEQ ID NO: 155), EVEA ORF 11 (SEQ ID NO: 157) and polypeptides having at least 99%, at least 95%, at least 90%, at least 85%, at least 80% or at least 70% homology to a polypeptide having the sequence of SEQ ID NOS: 153, 155, 157 or AVIL GenBank accession no. AAK83187 as determined using the BLASTP algorithm with the default parameters.

Members of protein family OXRW include polypeptides selected from AVIA ORF 33 (SEQ ID NO: 159), EVER ORF 26 (SEQ ID NO: 161), EVEA ORF 30 (SEQ ID NO: 163) and polypeptides having at least 99%, at least 95%, at least 90%, at least 85%, at least 80% or at least 70% homology to a polypeptide having the sequence of SEQ ID NOS: 159, 161 or 163 as determined using the BLASTP algorithm with the default parameters.

Members of protein family OXRW include polypeptides selected from AVIA ORF 19 (SEQ ID NO: 167), EVEA ORF 6 (SEQ ID NO: 173), AVIL GenBank accession no. AAK83181, EVER ORF 31 (SEQ ID NO: 169) and polypeptides having at least 99%, at least 95%, at least 90%, at least 85%, at least 80% or at least 70% homology to a polypeptide having the sequence of SEQ ID NOS: 167, 169, 173 or AVIL GenBank accession no. AAK83181 as determined using the BLASTP algorithm with the default parameters.

Members of protein family PHOD include polypeptides selected from AVIA ORF 34 (SEQ ID NO: 175), EVER ORF 33 (SEQ ID NO: 177), EVEA ORF 29 (SEQ ID NO: 179) and polypeptides having at least 99%, at least 95%, at least 90%, at least 85%, at least 80% or at least 70% homology to a polypeptide having the sequence of SEQ ID NOS: 175, 177 or 179 as determined using the BLASTP algorithm with the default parameters.

Members of protein family UNAJ include polypeptides selected from AVIA ORF 18 (SEQ ID NO: 165), EVEA ORF 5 (SEQ ID NO: 171), EVER ORF 31 (SEQ ID NO: 169) and polypeptides having at least 99%, at least 95%, at least 90%, at least 85%, at least 80% or at least 70% homology to a polypeptide having the sequence of SEQ ID NOS: 165, 169 or 171 as determined using the BLASTP algorithm with the default parameters.

Members of protein family UEVA include polypeptides selected from AVIA ORF 26 (SEQ ID NO: 193), AVIL GenBank accession no. AAK83189, EVER ORF 17 (SEQ ID NO: 195), EVEA ORF 14 (SEQ ID NO: 197) and polypeptides having at least 99%, at least 95%, at least 90%, at least 85%, at least 80% or at least 70% homology to a polypeptide having the sequence of SEQ ID NOS: 193, 195, 197 or AVIL GenBank accession no. AAK83189 as determined using the BLASTP algorithm with the default parameters.

Members of protein family UEVB include polypeptides selected from AVIA ORF 9 (SEQ ID NO: 199), AVIL GenBank accession no. AAK83174, EVER ORF 9 (SEQ ID NO: 201), and polypeptides having at least 99%, at least 95%, at least 90%, at least 85%, at least 80% or at least 70% homology to a polypeptide having the sequence of SEQ ID NOS: 199, 201 or AVIL GenBank accession no. AAK83174 as determined using the BLASTP algorithm with the default parameters.

Members of protein family UNKU include polypeptides selected from AVIA ORF 2 (SEQ ID NO: 203), EVER ORF 25 (SEQ ID NO: 205), EVEA ORF 32 (SEQ ID NO: 207) and polypeptides having at least 99%, at least 95%, at least 90%, at least 85%, at least 80% or at least 70% homology to a polypeptide having the sequence of SEQ ID NOS: 203, 205 or 207 as determined using the BLASTP algorithm with the default parameters.

Structural features that distinguish everninomicin-type orthosomycins from other orthosomycins require one or more proteins selected from a group of nine protein families, namely DATC, DEPF, EPIM, GTFA, MTFG, MTFV, OXBN, OXCO and UNBB. A polypeptide representing a member of any one of these nine protein families or a polynucleotide encoding a polypeptide representing a member of any one of these nine protein families is considered diagnostic of an everninomicin-type orthosomycin gene cluster and an everninomicin-type orthosomycin producing organism. In a preferred embodiment, a polypeptide representing a member of any one of these nine protein families, i.e. DATC, DEPF, EPIM, GTFA, MTFG, MTFV, OXBN, OXCO and UNBB, or a polynucleotide encoding a polypeptide representing a member of these nine protein families is detected together with one or more polypeptides representing a member of any one of the seventeen protein families diagnostic of an orthosomycin biosynthetic gene cluster, i.e. GTFE, GTFG, GTFH, HOXG, MTFD, MTFE, MTFF, MTLA, MTIA, OXRV, OXRW, OXRW, PHOD, UNAJ, UEVA, UEVB and UNKU or one or more polynucleotides encoding a polypeptide representing a member of these seventeen protein families.

It is not expected that an everninomicin-type orthosomycin biosynthetic locus will contain a member of each of the nine protein families considered diagnostic of everninomicin-type orthosomycin biosynthetic loci. Rather, the presence of at least one, preferably two, more preferably three, still more preferably four, and most preferably six or more of the nine protein families DATC, DEPF, EPIM, GTFA, MTFG, MTFV, OXBN, OXCO and UNBB indicates the presence of an everninomicin-type orthosomycin biosynthetic locus and an everninomicin-type orthosomycin producing organism. In a preferred embodiment, the presence of at least one, preferably two, more preferably three, still more preferably four, and most preferably six or more of the nine protein families DATC, DEPF, EPIM, GTFA, MTFG, MTFV, OXBN, OXCO and UNBB, detected together with the presence of at least one, preferably 2, more preferably 4, still more preferably 6, still more preferably 8 still more preferably 10 or more of the seventeen protein families diagnostic of an orthosomycin biosynthetic gene cluster, i.e. GTFE, GTFG, GTFH, HOXG, MTFD, MTFE, MTFF, MTLA, MTIA, OXRV, OXRW, OXRW, PHOD, UNAJ, UEVA, UEVB and UNKU indicates the presence of an everninomicin-type orthosomycin biosynthetic locus and an everninomicin-type orthosomycin producing organism.

Members of the protein family DATC include polypeptides selected from EVER ORF 43 (SEQ ID NO: 209), EVEA ORF 37 (SEQ ID NO: 211) and polypeptides having at least 99%, at least 95%, at least 90%, at least 85%, at least 80% or at least 70% homology to a polypeptide of EVER ORF 43 (SEQ ID NO: 209) or EVEA ORF 37 (SEQ ID NO: 211) as determined using the BLASTP algorithm with the default parameters.

Members of the protein family DEPF include polypeptides selected from EVER ORF 46 (SEQ ID NO: 213), EVEA ORF 40 (SEQ ID NO: 215) and polypeptides having at least 99%, at least 95%, at least 90%, at least 85%, at least 80% or at least 70% homology to a polypeptide of EVER ORF 46 (SEQ ID NO: 213) or EVEA ORF 40 (SEQ ID NO: 215) as determined using the BLASTP algorithm with the default parameters.

Members of the protein family EPIM include polypeptides selected from EVER ORF 45 (SEQ ID NO: 217), EVEA ORF 39 (SEQ ID NO: 219) and polypeptides having at least 99%, at least 95%, at least 90%, at least 85%, at least 80% or at least 70% homology to a polypeptide of EVER ORF 45 (SEQ ID NO: 217) or EVEA ORF 39 (SEQ ID NO: 219) as determined using the BLASTP algorithm with the default parameters.

Members of the protein family GTFA include polypeptides selected from EVER ORF 21 (SEQ ID NO: 221), EVEA ORF 35 (SEQ ID NO: 223) and polypeptides having at least 99%, at least 95%, at least 90%, at least 85%, at least 80% or at least 70% homology to a polypeptide of EVER ORF 21 (SEQ ID NO: 221) or EVEA ORF 35 (SEQ ID NO: 223) as determined using the BLASTP algorithm with the default parameters.

Members of protein family MTFG include polypeptides selected from EVER ORF 3 (SEQ ID NO: 225), EVEA ORF 18 (SEQ ID NO: 227), and polypeptides having at least 99%, at least 95%, at least 90%, at least 85%, at least 80% or at least 70% homology to a polypeptide of EVER ORF 3 (SEQ ID NO: 225) or EVEA ORF 18 (SEQ ID NO: 227) as determined using the BLASTP algorithm with the default parameters.

Members of protein family MTFV include polypeptides selected from EVER ORF 44 (SEQ ID NO: 229), EVEA ORF 38 (SEQ ID NO: 231) and polypeptides having at least 99%, at least 95%, at least 90%, at least 85%, at least 80% or at least 70% homology to a polypeptide of EVER ORF 44 (SEQ ID NO: 229) or EVEA ORF 38 (SEQ ID NO: 231) as determined using the BLASTP algorithm with the default parameters.

Members of protein family OXBN include polypeptides selected from EVER ORF 42 (SEQ ID NO: 233), EVEA 36 (SEQ ID NO: 235) and polypeptides having at least 99%, at least 95%, at least 90%, at least 85%, at least 80% or at least 70% homology to a polypeptide of EVER ORF 42 (SEQ ID NO: 233) or EVEA 36 (SEQ ID NO: 235) as determined using the BLASTP algorithm with the default parameters.

Members of protein family OXCO include polypeptides selected from EVER ORF 4 (SEQ ID NO: 237), EVEA ORF 19 (SEQ ID NO: 239) and polypeptides having at least 99%, at least 95%, at least 90%, at least 85%, at least 80% or at least 70% homology to a polypeptide of EVER ORF 4 (SEQ ID NO: 237) or EVEA ORF 19 (SEQ ID NO: 239) as determined using the BLASTP algorithm with the default parameters.

Members of protein family UNBB include polypeptides selected from EVER ORF 47 (SEQ ID NO: 241), EVEA ORF 41 (SEQ ID NO: 243) and polypeptides having at least 99%, at least 95%, at least 90%, at least 85%, at least 80% or at least 70% homology to a polypeptide of EVER ORF 47 (SEQ ID NO: 241) or EVEA ORF 41 (SEQ ID NO: 243) as determined using the BLASTP algorithm with the default parameters.

Structural features that distinguish avilamycin-type orthosomycins from other orthosomycins involve one or more proteins selected from a group of six protein families, namely ABCD, DEPN, MEMD, REBU, UNAI and UNBR. A polypeptide representing a member of any one of these six protein families or a polynucleotide encoding a polypeptide representing a member of any one or these six protein families is considered diagnostic of an avilamycin-type orthosomycin gene cluster and an avilamycin-type orthosomycin producing organism. In a preferred embodiment, a polypeptide representing a member of any one of these six protein families, i.e. ABCD, DEPN, MEMD, REBU, UNAI and UNBR or a polynucleotide encoding a polypeptide representing a member of these six protein families is detected together with one or more polypeptides representing a member of any one of the seventeen protein families diagnostic of an orthosomycin biosynthetic gene cluster, i.e. GTFE, GTFG, GTFH, HOXG, MTFD, MTFE, MTFF, MTLA, MTIA, OXRV, OXRW, OXRW, PHOD, UNAJ, UEVA, UEVB and UNKU or one or more polynucleotides encoding a polypeptide representing a member of these seventeen protein families.

It is not expected that an avilamycin-type orthosomycin biosynthetic locus will contain a member of each of the six protein families considered diagnostic of avilamycin-type orthosomycin biosynthetic loci. Rather, the presence of at least one, preferably two, more preferably three, still more preferably four, and most preferably five or six of the protein families ABCD, DEPN, MEMD, REBU, UNAI and UNBR indicates the presence of an avilamycin-type orthosomycin biosynthetic locus and an avilamycin-type orthosomycin producing organism. In a preferred embodiment, the presence of at least one, preferably two, more preferably three, still more preferably four, and most preferably five or six of the protein families ABCD, DEPN, MEMD, REBU, UNAI and UNBR, detected together with the presence of at least one, preferably 2, more preferably 4, still more preferably 6, still more preferably 8 still more preferably 10 or more of the seventeen protein families diagnostic of an orthosomycin biosynthetic gene cluster, i.e. GTFE, GTFG, GTFH, HOXG, MTFD, MTFE, MTFF, MTLA, MTIA, OXRV, OXRW, OXRW, PHOD, UNAJ, UEVA, UEVB and UNKU indicates the presence of an avilamycin-type orthosomycin biosynthetic locus and an avilamycin-type orthosomcyin producing organism.

Members of protein family ABCD include polypeptides selected from AVIA ORF 27 (SEQ ID NO: 245), AVIL GenBank accession no. AAG32068 and polypeptides having at least 99%, at least 95%, at least 90%, at least 85%, at least 80% or at least 70% homology to a polypeptide of AVIA ORF 27 (SEQ ID NO: 245) or AVIL GenBank accession no. AAG32068 as determined using the BLASTP algorithm with the default parameters.

Members of protein family DEPN include polypeptides selected from AVIA ORF 21 (SEQ ID NO: 247), AVIL GenBank accession no. AAK83183, and polypeptides having at least 99%, at least 95%, at least 90%, at least 85%, at least 80% or at least 70% homology to a polypeptide of AVIA ORF 21 (SEQ ID NO: 247) or AVIL GenBank accession no. AAK83183 as determined using the BLASTP algorithm with the default parameters.

Members of the protein family MEMD include polypeptides selected from AVIA ORF 28 (SEQ ID NO: 249), AVIL GenBank accession no. AAG32069, and polypeptides having at least 99%, at least 95%, at least 90%, at least 85%, at least 80% or at least 70% homology to a polypeptide of AVIA ORF 28 (SEQ ID NO: 249) or AVIL GenBank accession no. AAG32069 as determined using the BLASTP algorithm with the default parameters.

Members of the protein family REBU include polypeptides selected from AVIA ORF 7 (SEQ ID NO: 251), AVIL GenBank accession no. AAK83172, and polypeptides having at least 99%, at least 95%, at least 90%, at least 85%, at least * 80% or at least 70% homology to a polypeptide of AVIA ORF 7 (SEQ ID NO: 251) or AVIL GenBank accession no. AAK83172 as determined using the BLASTP algorithm with the default parameters.

Members of the protein family UNAI include polypeptides selected from AVIA ORF 6 (SEQ ID NO: 253), AVIL GenBank accession no. AAK83171 and polypeptides having at least 99%, at least 95%, at least 90%, at least 85%, at least 80% or at least 70% homology to a polypeptide of AVIA ORF 6 (SEQ ID NO: 253) or AVIL GenBank accession no. AAK83171 as determined using the BLASTP algorithm with the default parameters.

Members of the protein family UNBR include polypeptides selected from AVIA ORF 10 (SEQ ID NO: 255), AVIL GenBank accession no. AAK83175, and polypeptides having at least 99%, at least 95%, at least 90%, at least 85%, at least 80% or at least 70% homology to a polypeptide of AVIA ORF 10 (SEQ ID NO: 255) or AVIL GenBank accession no. AAK83175 as determined using the BLASTP algorithm with the default parameters.

Hybridization Probes and PCR Primers:

To identify an orthosomycin-producing organism or an orthosomycin biosynthetic locus, nucleic acids from cultivated microorganisms or from an environmental sample, e.g. soil, potentially harboring an organism having the genetic capacity to produce an orthosomycin compound may be contacted with a probe based on nucleotide sequences coding a member of the 17 protein families associated with biosynthesis of the structural features common to orthosomycins. Useful probes may be designed based on a nucleic acid or a combination of nucleic acids selected from the group consisting of (1) a nucleic acid sequence encoding a polypeptide of the GTFE family, for example a nucleic acid of SEQ ID NOS: 52, 54, 56, (the nucleic acid sequences coding for the GTFE protein in AVIA ORF 31, EVER ORF 24 and EVEA ORF 33 respectively) or the nucleic acid sequence coding for AVIL GenBank accession no. AAK83192; (2) a nucleic acid sequence encoding a polypeptide of the GTFG family, for example a nucleic acid of SEQ ID NOS: 58, 60, 62 (the nucleic acid sequences coding for the GTFG protein in AVIA ORF 5, EVER ORF 35 and EVEA ORF 27 respectively) or the nucleic acid sequence coding for AVIL GenBank accession no. AAK83170; (3) a nucleic acid sequence encoding a polypeptide of the GTFH family, for example a nucleic acid of SEQ ID NOS: 64, 66, 68 (the nucleic acid sequences coding for the GTFH protein in AVIA ORF 32, EVER ORF 8 and EVEA ORF 31 respectively) or the nucleic acid sequence coding for AVIL GenBank accession no. AAK83193; (4) a nucleic acid sequence encoding a polypeptide of the HOXG family, for example a nucleic acid of SEQ ID NOS: 70, 72, 74 (the nucleic acid sequences coding for the HOXG protein in AVIA ORF37, EVER ORF 12 and EVEA ORF 43 respectively); (5) a nucleic acid sequence encoding a polypeptide of the MTFD family, for example a nucleic acid of SEQ ID NOS: 100, 102, 104 (the nucleic acid sequences coding for the MTFD protein in AVIA ORF 22, EVER ORF 15 and EVEA ORF 8 respectively) or the nucleic acid sequence coding for AVIL GenBank accession no. AAK83184; (6) a nucleic acid sequence encoding a polypeptide of the MTFE family, for example a nucleic acid of SEQ ID NOS: 106, 108, 110 (the nucleic acid sequences coding for the MTFE protein in AVIA ORF 23, EVER ORF 19 and EVEA ORF 10 respectively) or the nucleic acid sequence coding for AVIL GenBank accession no. AAK83186; (7) a nucleic acid sequence encoding a polypeptide of the MTFF family, for example a nucleic acid of SEQ ID NOS: 112, 114, 116 (the nucleic acid sequences coding for the MTFF protein in AVIA ORF 25, EVER ORF 5 and EVEA ORF 12 respectively) or the nucleic acid sequence coding for AVIL GenBank accession no. AAK83188; (8) a nucleic acid sequence encoding a polypeptide of the MTLA family, for example a nucleic acid of SEQ ID NOS: 128, 130, 132 (the nucleic acid sequences coding for the MTLA protein in AVIA ORF 3, EVER ORF 40 and EVEA ORF 45 respectively) or the nucleic acid sequence coding for AVIL GenBank accession no. AAG32067; (9) a nucleic acid sequence encoding a polypeptide of the MTIA family, for example a nucleic acid of SEQ ID NOS: 124, 126 (the nucleic acid sequences coding for the MTIA protein in AVIA ORF 1 and EVER ORF 13 respectively) or the nucleic acid sequence coding for AVIL GenBank accession no. AAG32066; (10) a nucleic acid sequence encoding a polypeptide of the OXRV family, for example a nucleic acid of SEQ ID NOS: 154, 156, 158 (the nucleic acid sequences coding for the OXRV protein in AVIA ORF 24, EVER ORF 18 and EVEA ORF 11 respectively) or the nucleic acid sequence coding for AVIL GenBank accession no. AAK83187; (11) a nucleic acid sequence encoding a polypeptide of the OXRW family, for example a nucleic acid of SEQ ID NOS: 160, 162 and 164 (the nucleic acid sequences coding for the OXRW protein in AVIA ORF 33, EVER ORF 26 and EVEA ORF 30 respectively); (12) a nucleic acid sequence encoding a polypeptide of the OXRW/OXRX family, for example a nucleic acid of SEQ ID NOS: (the nucleic acid sequences coding for the second OXRW protein in AVIA ORF 19, SEQ ID NO: 167; EVEA ORF 6; SEQ ID NO: 173, respectively), SEQ ID NO: 170 (the nucleic acid coding the OXRX protein in EVER ORF 31, and the nucleic acid sequence coding for AVIL GenBank accession no. AAK83181; (13) a nucleic acid sequence encoding a polypeptide of the PHOD family, for example a nucleic acid of SEQ ID NOS: 176, 178 and 180 (the nucleic acid sequences coding for the PHOD protein in AVIA ORF 34, EVER ORF 33 and EVEA ORF 29 respectively); (14) a nucleic acid sequence encoding a polypeptide of the UNAJ/OXRX family, for example a nucleic acid of SEQ ID NOS: (the nucleic acid sequences coding for the UNAJ protein in AVIA ORF 18, SEQ ID NO: 165, and EVEA ORF 5, SEQ ID NO: 171, respectively), SEQ ID NO: 170 (the nucleic acid coding the OXRX protein in EVER ORF 31); (15) a nucleic acid sequence encoding a polypeptide of the UEVA family, for example a nucleic acid of SEQ ID NOS: 194, 196 and 198 (the nucleic acid sequences coding for the UEVA protein in AVIA ORF 26, EVER ORF 17 and EVEA ORF 14 respectively) or the nucleic acid sequence coding for AVIL GenBank accession no. AAK83189; (16) a nucleic acid sequence encoding a polypeptide of the UEVB family, for example a nucleic acid of SEQ ID NOS: 200 and 202 (the nucleic acid sequences coding for the UEVB protein in AVIA ORF 9, and EVER ORF 9 respectively) or the nucleic acid sequence coding for AVIL GenBank accession no. AAK83174; (17) a nucleic acid sequence encoding a polypeptide of the UNKU family, for example a nucleic acid of SEQ ID NOS: 204, 206, 208 (the nucleic acid sequences coding for the UNKU protein in AVIA ORF 2, EVER ORF 25 and EVEA ORF 32 respectively). Preferred probes are isolated, purified or enriched nucleic acids derived from SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 and the sequences complementary thereto, or a fragment comprising at least 10, 15, 20, 25, 30, 40, 50, 75, 100, 150, 200, 300, 400 or 500 consecutive bases of one of the sequences of SEQ ID NO: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 or the sequences complementary thereto.

In such procedures, nucleic acids are obtained from cultivated microorganisms or from an environmental sample potentially harboring an organism having the genetic capacity to produce an orthosomycin compound. The nucleic acids are contacted with probes designed based on the teachings and compositions of the invention under conditions which permit the probe to specifically hybridize to any complementary sequences indicative of the presence of an orthosomycin-specific protein family. The presence of at least two, preferably five, more preferably 8, still more preferably 10 or more of the seventeen orthosomycin specific protein families indicates the presence of an orthosomycin biosynthetic locus or an orthosomycin producing organism.

Diagnostic nucleic acid sequences for identifying orthosomycin genes, biosynthetic loci, and microorganisms that harbor such genes or loci may be employed on complex mixtures of microorganisms such as those from environmental samples (e.g., soil). A mixture of microorganisms refers to a heterogeneous population of microorganisms consisting of more than one species or strain. In the absence of amplification outside of its natural habitat, such a mixture of microorganisms is said to be uncultured. A cultured mixture of microorganisms may be obtained by amplification or propagation outside of its natural habitat by in vitro culture using various growth media that provide essential nutrients. However, depending on the growth medium used, the amplification may preferentially result in amplification of a sub-population of the mixture and hence may not be always desirable. If desired, a pure culture representing a single species or strain may obtained from either a cultured or uncultured mixture of microorganisms by established microbiological techniques such as serial dilution followed by growth on solid media so as to isolate individual colony forming units.

Orthosomycin genes and/or orthosomycin biosynthetic loci may be identified from either a pure culture or cultured or uncultured mixtures of microorganisms employing the diagnostic nucleic acid sequences disclosed in this invention by experimental techniques such as PCR, hybridization, or shotgun sequencing followed by bioinformatic analysis of the sequence data. The identification of orthosomycin genes and/or an orthosomycin biosynthetic locus in a pure culture of a single organism directly distinguishes such an organism with the genetic potential to produce a natural compound or multiple natural compounds belonging to the orthosomycin class. The identification of orthosomycin genes and/or orthosomycin biosynthetic loci in a cultured or uncultured mixture of microorganisms requires further steps to identify and isolate the microorganism(s) that harbor(s) them so as to obtain pure cultures of such microorganisms. One general method that might be employed to identify microorganisms that harbour orthosomycin genes and/or orthosomycin biosynthetic loci from a cultured mixture of microorganisms is the colony lift technique (Ausubel et al., Current Protocols in Molecular Biology, John Wiley 503 Sons, Inc. 1997; and Sambrook et al., Molecular Cloning: A Laboratory Manual 2d Ed., Cold Spring Harbor Laboratory Press, 1989) in which the mixture is grown on an appropriate solid medium, the resulting colony forming units are replicated on a solid matrix such as a nylon membrane, the membrane is contacted with detectable diagnostic nucleic acid sequences, the positive colony forming units are identified, and the corresponding colony forming units on the original medium are identified, purified, and amplified.

The orthosomycin diagnostic nucleic acids may be used to survey a number of environmental samples for the presence of organisms that have the potential to produce orthosomycin compounds, i.e., those organisms that contain orthosomycin genes and/or orthosomycin biosynthetic loci. One protocol for use of a survey to identify a polypeptide from DNA isolated from uncultured mixtures of microorganisms is outlined in Seow et al. (1997) J. Bacteriol. Vol.179 pp. 7360-7368.

To identify an everninomicin-type orthosomycin producer or an everninomicin-type orthosomycin biosynthetic gene cluster, nucleic acids from an environmental sample, e.g. soil, potentially harboring an organism having the genetic capacity to produce an everninomicin-type orthosomycin compound may further contacted be with a probe constructed based on a nucleotide sequence corresponding to the protein families associated with the structural features unique to everninomicin-type orthosomycins. Useful probes may be designed based on a nucleic acid selected from the group consisting of (1) a nucleic acid sequence encoding a polypeptide of the DATC family, for example a nucleic acid of SEQ ID NOS: 210, 212 (the nucleic acid sequences coding for the DATC protein in EVER ORF 43 and EVEA ORF 37 respectively); (2) a nucleic acid sequence encoding a polypeptide of the DEPF family, for example a nucleic acid of SEQ ID NOS: 214, 216 (the nucleic acid sequences coding for the DEPF protein in EVER ORF 46 and EVEA ORF 40 respectively); (3) a nucleic acid sequence encoding a polypeptide of the EPIM family, for example a nucleic acid of SEQ ID NOS: 218 and 220 (the nucleic acid sequences coding for the EPIM protein in EVER ORF 45 and EVEA ORF 39 respectively); (4) a nucleic acid sequence encoding a polypeptide of the GTFA family, for example a nucleic acid of SEQ ID NOS: 222 and 224 (the nucleic acid sequences coding for the GTFA protein in EVER ORF 21 and EVEA ORF 35 respectively); (5) a nucleic acid sequence encoding a polypeptide of the MTFG family, for example a nucleic acid of SEQ ID NOS: 226, 228 (the nucleic acid sequences coding for the MTFG protein in EVER ORF 3 and EVEA ORF 18 respectively); (6) a nucleic acid sequence encoding a polypeptide of the MTFV family, for example a nucleic acid of SEQ ID NOS: 230, 232 (the nucleic acid sequences coding for the MTFV protein in EVER ORF 44 and EVEA ORF 38 respectively); (7) a nucleic acid sequence encoding a polypeptide of the OXBN family, for example a nucleic acid of SEQ ID NOS: 234 and 236 (the nucleic acid sequences coding for the OXBN protein in EVER ORF 42 and EVEA ORF 36 respectively); (8) a nucleic acid sequence encoding a polypeptide of the OXCO family, for example a nucleic acid of SEQ ID NOS: 238, 240 (the nucleic acid sequences coding for the OXCO protein in EVER ORF 4 and EVEA ORF 19 respectively); and (9) a nucleic acid sequence encoding a polypeptide of the UNBB family, for example a nucleic acid of SEQ ID NOS: 242, 244 (the nucleic acid sequences coding for the UNBB protein in EVER ORF 47 and EVEA ORF 41 respectively). Preferred probes are isolated, purified or enriched nucleic acid derived from SEQ ID NOS: 210, 212, 214, 216, 218, 220, 222, 224, 226, 228, 230, 232, 234, 236, 238, 240, 242, 244, and the sequences complementary thereto, or a fragment comprising at least 10, 15, 20, 25, 30, 40, 50, 75, 100, 150, 200, 300, 400 or 500 consecutive bases of one of the sequences of SEQ ID NOS: 210, 212, 214, 216, 218, 220, 222, 224, 226, 228, 230, 232, 234, 236, 238, 240, 242, 244 and the sequences complementary thereto.

In such procedures, nucleic acids are obtained from cultivated microorganisms or from an environmental sample potentially harboring an organism having the genetic capacity to produce an everninomicin-type orthosomycin compound. The environmental sample may be a mixture of microorganisms or a pure culture of a single microorganism. The nucleic acids are contacted with probes designed based on the teachings and compositions of the invention under conditions which permit the probe to specifically hybridize to any complementary sequences indicative of the presence of an everninomicin-type orthosomycin-specific protein family. The presence of at least one, preferably 2, more preferably 4, still more preferably 6 or more of the nine everninomicin-type orthosomycin specific protein families indicates the presence of an everninomicin-type orthosomycin biosynthetic locus and an everninomicin-type orthosomycin producing organism.

To identify an avilamycin-type orthosomycin producer or an avilamycin-type biosynthetic locus, nucleic acids from cultivated microorganisms or from an environmental sample, e.g. soil, potentially harboring an organism having the genetic capacity to produce an avilamycin-type orthosomycin compound is further contacted with a probe corresponding to a member of the six protein families associated with biosynthesis of the structural features common to avilamycin-type orthosomycins. Useful probes may be constructed from a nucleic acid selected from the group consisting of (1) a nucleic acid sequence encoding a polypeptide of the ABCD family, for example SEQ ID NO: 246 (AVIA ORF 27) or AVIL GenBank accession no. AAG32068; (2) a nucleic acid sequence encoding a polypeptide of the DEPN family, for example SEQ ID NO: 248 (AVIA ORF 21) or AVIL GenBank accession no. AAK83183; (3) a nucleic acid sequence encoding a polypeptide of the MEMD family, for example SEQ ID NO: 250 (AVIA ORF 28)-or AVIL GenBank accession no. AAG32069; (4) a nucleic acid sequence encoding a polypeptide of the REBU family, for example SEQ ID NO: 252 (AVIA ORF 7) or AVIL GenBank accession no. AAK83172; (5) a nucleic acid sequence encoding a polypeptide of the UNAI family, for example SEQ ID NO: 254 (AVIA ORF 6) or AVIL GenBank accession no. AAK83171; and (6) a nucleic acid sequence encoding a polypeptide of the UNBR family, for example SEQ ID NO: 256 (AVIA ORF 10) or AVIL GenBank accession no. AAK83175. Preferred probes are isolated, purified or enriched nucleic acid derived from SEQ ID NOS: 246, 248, 250, 252, 254, 256 and the nucleic acid sequences corresponding to Genbank accession nos. AAG32068, AAK83183, AAG32069, AAK83172, AAK83171 and AAK83175, the sequences complementary thereto, or a fragment comprising at least 10, 15, 20, 25, 30, 40, 50, 75, 100, 150, 200, 300, 400 or 500 consecutive bases of one of the sequences of SEQ ID NOS: 246, 248, 250, 252, 254, 256 and the sequences complementary thereto.

In such procedures, nucleic acids are obtained from cultivated microorganisms or from an environmental sample potentially harboring an organism having the genetic capacity to produce an avilamycin-type orthosomycin compound. The environmental sample may be a mixture of microorganisms or a pure culture of a single microorganism. The nucleic acids are contacted with probes designed based on the teachings and compositions of the invention under conditions which permit the probe to specifically hybridize to any complementary sequences indicative of the presence of an avilamycin-type orthosomycin-specific protein family. The presence of at least one, preferably 2, more preferably 3, still more preferably 4 or more of the six avilamycin-type orthosomycin specific protein families indicates the presence of an avilamycin-type orthosomycin biosynthetic locus and an avilamycin-type orthosomycin producing organism.

Where necessary, conditions which permit the probe to specifically hybridize to complementary sequences from an orthosomycin-producer may be determined by placing the probe in contact with complementary sequences obtained from an orthosomycin-producer as well as control sequences which are not from an orthosomycin-producer. In some analyses, the control sequences may be from organisms related to orthosomycin-producers. Alternatively, the control sequences are not related to orthosomycin-producers. Hybridization conditions, such as the salt concentration of the hybridization buffer, the formamide concentration of the hybridization buffer, or the hybridization temperature, may be varied to identify conditions which allow the probe to hybridize specifically to nucleic acids from orthosomycin-producers.

If the sample contains nucleic acids from orthosomycin-producers, specific hybridization of the probe to the nucleic acids from the orthosomycin-producer is then detected. Hybridization may be detected by labeling the probe with a detectable agent such as a radioactive isotope, a fluorescent dye or an enzyme capable of catalyzing the formation of a detectable product.

Many methods of using the labeled probes to detect the presence of nucleic acids from an orthosomycin-producer in a sample are familiar to those skilled in the art. These include Southern Blots, Northern Blots, colony hybridization procedures, and dot blots. Protocols for each of these procedures are provided in Ausubel et al., Current Protocols in Molecular Biology, John Wiley 503 Sons, Inc. 1997; and Sambrook et al., Molecular Cloning: A Laboratory Manual 2d Ed., Cold Spring Harbor Laboratory Press, 1989.

Alternatively, more than one probe designed based on the teachings and compositions of the invention may be used in an amplification reaction to determine whether the nucleic acid sample contains nucleic acids from an orthosomycin-producer. Preferably the probes comprise oligonucleotides. In one embodiment, the amplification reaction may comprise a Polymerase Chain Reaction (PCR) reaction. PCR protocols are described in Ausubel and Sambrook, supra. In such procedures, the nucleic acids in the sample are contacted with the probes, the amplification reaction is performed, and any amplification product is detected. The amplification product may be detected by performing gel electrophoresis on the reaction products and staining the gel with an interculator such as ethidium bromide. Alternatively, one or more of the probes may be labeled with a radioactive isotope and the presence of a radioactive isotope and the presence of a radioactive amplification product may be detected by autoradiography after gel electrophoresis.

The isolated, purified or enriched nucleic acids of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, the sequences complementary thereto, or a fragment comprising at least 10, 15, 20, 25, 30, 35, 40, 50, 75, 100, 150, 200, 300, 400 or 500 consecutive bases of one of the sequence of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, or the sequences complementary thereto may be used as probes to identify and isolate DNAs encoding the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255 respectively. In such procedures, a genomic DNA library is constructed from a sample containing an orthosomycin producer. The genomic DNA library is then contacted with a probe comprising a coding sequence or a fragment of the coding sequence, encoding one of the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, or a fragment thereof under conditions which permit the probe to specifically hybridize to sequences complementary thereto. In a preferred embodiment, the probe is an oligonucleotide of about 10 to about 30 nucleotides in length designed based on a nucleic acid of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256,. Genomic DNA clones which hybridize to the probe are then detected and isolated. Procedures for preparing and identifying DNA clones of interest are disclosed in Ausubel et al., Current Protocols in Molecular Biology, John Wiley 503 Sons, Inc. 1997; and Sambrook et al., Molecular Cloning: A Laboratory Manual 2d Ed., Cold Spring Harbor Laboratory Press, 1989.

The isolated, purified or enriched nucleic acids of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, or the sequences complementary thereto may be used as probes to identify and isolate related nucleic acids. In some embodiments, the related nucleic acids may be genomic DNAs (or cDNAs) from potential orthosomycin producers. In such procedures, a nucleic acid sample containing nucleic acids from a potential orthosomycin-producer is contacted with the probe under conditions which permit the probe to specifically hybridize to related sequences. The nucleic acid sample may be a genomic DNA (or cDNA) library from the potential orthosomycin-producer. Hybridization of the probe to nucleic acids is then detected using any of the methods described above.

Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10× Denhardt's, and 0.5 mg/ml polyriboadenylic acid. Approximately 2×10⁷ cpm (specific activity 4-9×10⁸ cpm/ug) of ³²P end-labeled oligonucleotide probe are then added to the solution. After 12-16 hours of incubation, the membrane is washed for 30 minutes at room temperature in 1× SET (150 mM NaCl, 20 mM Tris hydrochloride, pH 7.8, 1 mM Na₂EDTA) containing 0.5% SDS, followed by a 30 minute wash in fresh 1× SET at Tm-10 C for the oligonucleotide probe where Tm is the melting temperature. The membrane is then exposed to auto-radiographic film for detection of hybridization signals.

By varying the stringency of the hybridization conditions used to identify nucleic acids, such as genomic DNAs or cDNAs, which hybridize to the detectable probe, nucleic acids having different levels of homology to the probe can be identified and isolated. Stringency may be varied by conducting the hybridization at varying temperatures below the melting temperatures of the probes. The melting temperature of the probe may be calculated using the following formulas:

For oligonucleotide probes between 14 and 70 nucleotides in length the melting temperature (Tm) in degrees Celcius may be calculated using the formula: Tm=81.5+16.6(log [Na+])+0.41 (fraction G+C)−(600/N) where N is the length of the oligonucleotide.

If the hybridization is carried out in a solution containing formamide, the melting temperature may be calculated using the equation Tm=81.5+16.6(log [Na +])+0.41 (fraction G+C)−(0.63% formamide)−(600/N) where N is the length of the probe.

Prehybridization may be carried out in 6× SSC, 5× Denhardt's reagent, 0.5% SDS, 0.1 mg/ml denatured fragmented salmon sperm DNA or 6× SSC, 5× Denhardt's reagent, 0.5% SDS, 0.1 mg/ml denatured fragmented salmon sperm DNA, 50% formamide. The composition of the SSC and Denhardt's solutions are listed in Sambrook et al., supra.

Hybridization is conducted by adding the detectable probe to the hybridization solutions listed above. Where the probe comprises double stranded DNA, it is denatured by incubating at elevated temperatures and quickly cooling before addition to the hybridization solution. It may also be desirable to similarly denature single stranded probes to eliminate or diminish formation of secondary structures or oligomerization. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. Preferably, the hybridization is conducted in 6× SSC, for shorter probes. Preferably, the hybridization is conducted in 50% formamide containing solutions, for longer probes.

All the foregoing hybridizations would be considered to be examples of hybridization performed under conditions of high stringency.

Following hybridization, the filter is washed for at least 15 minutes in 2× SSC, 0.1% SDS at room temperature or higher, depending on the desired stringency. The filter is then washed with 0.1× SSC, 0.5% SDS at room temperature (again) for 30 minutes to 1 hour.

Nucleic acids which have hybridized to the probe are identified by autoradiography or other conventional techniques.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1 M. Following hybridization, the filter may be washed with 2× SSC, 0.5% SDS at the temperature of hybridization. These conditions are considered to be “moderate stringency” conditions above 50° C. and “low stringency” conditions below 50° C. A specific example of “moderate stringency” hybridization conditions is when the above hybridization is conducted at 55° C. A specific example of “low stringency” hybridization conditions is when the above hybridization is conducted at 45° C.

Alternatively, the hybridization may be carried out in buffers, such as 6× SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6× SSC, 0.5% SDS at 50° C. These conditions are considered to be “moderate stringency” conditions above 25% formamide and “low stringency” conditions below 25% formamide. A specific example of “moderate stringency” hybridization conditions is when the above hybridization is conducted at 30% formamide. A specific example of “low stringency” hybridization conditions is when the above hybridization is conducted at 10% formamide.

Nucleic acids which have hybridized to the probe are identified by autoradiography or other conventional techniques.

For example, the preceding methods may be used to isolate nucleic acids having a sequence with at least 97%, at least 95%, at least 90%, at least 85%, at least 80%, or at least 70% homology to a nucleic acid sequence selected from the group consisting of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, fragments comprising at least 10, 15, 20, 25, 30, 35, 40, 50, 75, 100, 150, 200, 300, 400, or 500 consecutive bases thereof, and the sequences complementary thereto. Homology may be measured using BLASTN version 2.0 with the default parameters. For example, the homologous polynucleotides may have a coding sequence which is a naturally occurring allelic variant of one of the coding sequences described herein. Such allelic variant may have a substitution, deletion or addition of one or more nucleotides when compared to the nucleic acids of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, or the sequences complementary thereto.

Additionally, the above procedures may be used to isolate nucleic acids which encode polypeptides having at least 99%, 95%, at least 90%, at least 85%, at least 80%, or at least 70% homology to a polypeptide having the sequence of one of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, or fragments comprising at least 5, 10, 15, 20, 25, 30, 35, 40, 50, 75, 100, or 150 consecutive amino acids thereof as determined using the BLASTP version 2.2.2 algorithm with default parameters.

Bioinformatics:

As used herein, the term “orthosomycin-specific nucleic acid codes” encompass the nucleotide sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, fragments of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, nucleotide sequences homologous to SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, or homologous to fragments of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, and sequences complementary to all of the preceding sequences. The fragments include portions of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 comprising at least 10, 15, 20, 25, 30, 35, 40, 50, 75, 100, 150, 200, 300, 400 or 500 consecutive nucleotides of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208. Preferably, the fragments are novel fragments. Homologous sequences and fragments of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 refer to a sequence having at least 99%, 98%, 97%, 96%, 95%, 90%, 80%, 75% or 70% homology to these sequences. Homology may be determined using any of the computer programs and parameters described herein, including BLASTN and TBLASTX with the default parameters. Homologous sequences also include RNA sequences in which uridines replace the thymines in the nucleic acid codes of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208. The homologous sequences may be obtained using any of the procedures described herein or may result from the correction of a sequencing error. It will be appreciated that the nucleic acid codes of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 can be represented in the traditional single character format in which G, A, T and C denote the guanine, adenine, thymine and cytosine bases of the deoxyribonucleic acid (DNA) sequence respectively, or in which G, A, U and C denote the guanine adenine, uracil and cytosine bases of the ribonucleic acid (RNA) sequence (see the inside back cover of Stryer, Biochemistry, 3^{rd} edition, W. H. Freeman & Co., New York) or in any other format which records the identity of the nucleotides in a sequence.

The term “everninomicin-specific nucleic acid codes” encompass the nucleotide sequences of SEQ ID NOS: 210, 212, 214, 216, 218, 220, 222, 224, 226, 228, 230, 232, 234, 236, 238, 240, 242, 244, fragments of SEQ ID NOS: 210, 212, 214, 216, 218, 220, 222, 224, 226, 228, 230, 232, 234, 236, 238, 240, 242, 244, nucleotide sequences homologous to SEQ ID NOS: 210, 212, 214, 216, 218, 220, 222, 224, 226, 228, 230, 232, 234, 236, 238, 240, 242, 244, or homologous to fragments of SEQ ID NOS: 210, 212, 214, 216, 218, 220, 222, 224, 226, 228, 230, 232, 234, 236, 238, 240, 242, 244, and sequences complementary to all of the preceding sequences. The fragments include portions of SEQ ID NOS: 210, 212, 214, 216, 218, 220, 222, 224, 226, 228, 230, 232, 234, 236, 238, 240, 242, 244 comprising at least 10, 15, 20, 25, 30, 35, 40, 50, 75, 100, 150, 200, 300, 400 or 500 consecutive nucleotides of SEQ ID NOS: 210, 212, 214, 216, 218, 220, 222, 224, 226, 228, 230, 232, 234, 236, 238, 240, 242, 244. Preferably, the fragments are novel fragments. Homologous sequences and fragments of SEQ ID NOS: 210, 212, 214, 216, 218, 220, 222, 224, 226, 228, 230, 232, 234, 236, 238, 240, 242, 244, refer to a sequence having at least 99%, 98%, 97%, 96%, 95%, 90%, 80%, 75% or 70% homology to these sequences. Homology may be determined using any of the computer programs and parameters described herein, including BLASTN and TBLASTX with the default parameters. Homologous sequences also include RNA sequences in which uridines replace the thymines in the nucleic acid codes of SEQ ID NOS: 210, 212, 214, 216, 218, 220, 222, 224, 226, 228, 230, 232, 234, 236, 238, 240, 242, 244. The homologous sequences may be obtained using any of the procedures described herein or may result from the correction of a sequencing error. It will be appreciated that the nucleic acid codes of SEQ ID NOS: 210, 212, 214, 216, 218, 220, 222, 224, 226, 228, 230, 232, 234, 236, 238, 240, 242, 244 can be represented in the traditional single character format in which G, A, T and C denote the guanine, adenine, thymine and cytosine bases of the deoxyribonucleic acid (DNA) sequence respectively, or in which G, A, U and C denote the guanine adenine, uracil and cytosine bases of the ribonucleic acid (RNA) sequence (see the inside back cover of Stryer, Biochemistry, 3^{rd} edition, W. H. Freeman & Co., New York) or in any other format which records the identity of the nucleotides in a sequence.

The term “avilamycin-specific nucleic acid codes” encompass the nucleotide sequences of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; fragments of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; nucleotide sequences homologous to SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; or homologous to fragments of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; and sequences complementary to all of the preceding sequences. The fragments include portions of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175 comprising at least 10, 15, 20, 25, 30, 35, 40, 50, 75, 100, 150, 200, 300, 400 or 500 consecutive nucleotides of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175. Preferably, the fragments are novel fragments. Homologous sequences and fragments of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175 refer to a sequence having at least 99%, 98%, 97%, 96%, 95%, 90%, 80%, 75% or 70% homology to these sequences. Homology may be determined using any of the computer programs and parameters described herein, including BLASTN and TBLASTX with the default parameters. Homologous sequences also include RNA sequences in which uridines replace the thymines in the nucleic acid codes of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175. The homologous sequences may be obtained using any of the procedures described herein or may result from the correction of a sequencing error. It will be appreciated that the nucleic acid codes of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175 can be represented in the traditional single character format in which G, A, T and C denote the guanine, adenine, thymine and cytosine bases of the deoxyribonucleic acid (DNA) sequence respectively, or in which G, A, U and C denote the guanine adenine, uracil and cytosine bases of the ribonucleic acid (RNA) sequence (see the inside back cover of Stryer, Biochemistry, 3^{rd} edition, W. H. Freeman & Co., New York) or in any other format which records the identity of the nucleotides in a sequence.

“Orthosomycin-specific polypeptide codes” encompass the polypeptide sequences of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 which are encoded by the cDNAs of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208; polypeptide sequences homologous to the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207, or fragments of any of the preceding sequences. Homologous polypeptide sequences refer to a polypeptide sequence having at least 99%, 98%, 97%, 96%, 95%, 90%, 85%, 80%, 75% or 70% homology to one of the polypeptide sequences of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207. Polypeptide sequence homology may be determined using any of the computer programs and parameters described herein, including BLASTP version 2.2.2 with the default parameters or with any user-specified parameters. The homologous sequences may be obtained using any of the procedures described herein or may result from the correction of a sequencing error. The polypeptide fragments comprise at least 5, 10, 15, 20, 25, 30, 35, 40, 50, 75, 100 or 150 consecutive polypeptides of the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207. Preferably the fragments are novel fragments. It will be appreciated that the polypeptide codes of the SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 can be represented in the traditional single character format or three letter format (see the inside back cover of Stryer, Biochemistry, 3^{rd} edition, W.H. Freeman & Co., New York) or in any other format which relates the identity of the polypeptides in a sequence.

“Everninomicin-specific polypeptide codes” encompass the polypeptide sequences of SEQ ID NOS: 209, 211, 213, 215, 217, 219, 221, 223, 225, 227, 229, 231, 233, 235, 237, 239, 241 and 243 which are encoded by the cDNAs of SEQ ID NOS: 210, 212, 214, 216, 218, 220, 222, 224, 226, 228, 230, 232, 234, 236, 238, 240, 242, 244 and 244; polypeptide sequences homologous to the polypeptides of SEQ ID NOS: 209, 211, 213, 215, 217, 219, 221, 223, 225, 227, 229, 231, 233, 235, 237, 239, 241 and 243 or fragments of any of the preceding sequences. Homologous polypeptide sequences refer to a polypeptide sequence having at least 99%, 98%, 97%, 96%, 95%, 90%, 85%, 80%, 75% or 70% homology to one of the polypeptide sequences of SEQ ID NOS: 209, 211, 213, 215, 217, 219, 221, 223, 225, 227, 229, 231, 233, 235, 237, 239, 241 and 243. Polypeptide sequence homology may be determined using any of the computer programs and parameters described herein, including BLASTP version 2.2.2 with the default parameters or with any user-specified parameters. The homologous sequences may be obtained using any of the procedures described herein or may result from the correction of a sequencing error. The polypeptide fragments comprise at least 5, 10, 15, 20, 25, 30, 35, 40, 50, 75, 100 or 150 consecutive polypeptides of the polypeptides of SEQ ID NOS: 209, 211, 213, 215, 217, 219, 221, 223, 225, 227, 229, 231, 233, 235, 237, 239, 241 and 243. Preferably the fragments are novel fragments. It will be appreciated that the polypeptide codes of the SEQ ID NOS: 209, 211, 213, 215, 217, 219, 221, 223, 225, 227, 229, 231, 233, 235, 237, 239, 241 and 243 can be represented in the traditional single character format or three letter format (see the inside back cover of Stryer, Biochemistry, 3^{rd} edition, W.H. Freeman & Co., New York) or in any other format which relates the identity of the polypeptides in a sequence.

“Avilamycin-specific polypeptide codes encompass the polypeptide sequences of SEQ ID NOS: 245, 247, 249, 251, 253, 255 (encoded by the cDNAs of SEQ ID NOS: 246, 248, 250, 252, 254, 256) and the polypeptide sequences of GenBank accession nos: AAG32068, AAK83183, AAG32069, AAK83172, AAK83171 and AAK83175; polypeptide sequences homologous to the polypeptides of SEQ ID NOS: 245, 247, 249, 251, 253, 255 and to GenBank accession nos: AAG32068, AAK83183, AAG32069, AAK83172, AAK83171 and AAK83175 or fragments of any of the preceding sequences. Homologous polypeptide sequences refer to a polypeptide sequence having at least 99%, 98%, 97%, 96%, 95%, 90%, 85%, 80%, 75% or 70% homology to one of the polypeptide sequences of SEQ ID NOS: 245, 247, 249, 251, 253, 255 or to the polypeptides of GenBank accession nos: AAG32068, AAK83183, AAG32069, AAK83172, AAK83171 and AAK83175. Polypeptide sequence homology may be determined using any of the computer programs and parameters described herein, including BLASTP version 2.2.2 with the default parameters or with any user-specified parameters. The homologous sequences may be obtained using any of the procedures described herein or may result from the correction of a sequencing error. The polypeptide fragments comprise at least 5, 10, 15, 20, 25, 30, 35, 40, 50, 75, 100 or 150 consecutive polypeptides of the polypeptides of SEQ ID NOS: 245, 247, 249, 251, 253, 255 or to the polypeptides of GenBank accession nos: AAG32068, AAK83183, AAG32069, AAK83172, AAK83171 and AAK83175. Preferably the fragments are novel fragments. It will be appreciated that the polypeptide codes of SEQ ID NOS: 245, 247, 249, 251, 253, 255 and GenBank accession nos: AAG32068, AAK83183, AAG32069, AAK83172, AAK83171 and AAK83175 can be represented in the traditional single character format or three letter format (see the inside back cover of Stryer, Biochemistry, 3^{rd} edition, W. H. Freeman & Co., New York) or in any other format which relates the identity of the polypeptides in a sequence.

For ease of comprehension the orthosomycin-specific nucleic acid codes, the everninomicin-specific nucleic acid codes, the avilamycin-specific nucleic acid codes, the orthosomycin-specific polypeptide codes, the everninomicin-specific polypeptide codes and the avilamycin-specific polypeptide codes, or a subset thereof, are sometime collectively referred to as “the reference sequences”.

It will be readily appreciated by those skilled in the art that the reference sequences can be stored, recorded and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the the orthosomycin-specific nucleic acid codes, the everninomicin-specific nucleic acid codes, the avilamycin-specific nucleic acid codes, the orthosomycin-specific polypeptide codes, the everninomicin-specific polypeptide codes and the avilamycin-specific polypeptide codes.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of media known to those skilled in the art.

The orthosomycin-specific nucleic acid codes, the everninomicin-specific nucleic acid codes, the avilamycin-specific nucleic acid codes, the orthosomycin-specific polypeptide code, the everninomicin-specific polypeptide code, and the avilamycin-specific polypeptide codes may be stored and manipulated in a variety of data processor programs in a variety of formats. For example, the orthosomycin-specific nucleic acid codes, the everninomicin-specific nucleic acid codes, the avilamycin-specific nucleic acid codes, the orthosomycin-specific polypeptide code, the everninomicin-specific polypeptide code, and the avilamycin-specific polypeptide codes the nucleic acid codes may be stored as ASCII or text in a word processing file, such as MicrosoftWORD or WORDPERFECT in a variety of database programs familiar to those of skill in the art, such as DB2 or ORACLE. In addition, many computer programs and databases may be used as sequence comparers, identifiers or sources of query nucleotide sequences or query polypeptide sequences to be compared to the orthosomycin-specific nucleic acid codes, the everninomicin-specific nucleic acid codes, the avilamycin-specific nucleic acid codes, the orthosomycin-specific polypeptide code, the everninomicin-specific polypeptide code, and the avilamycin-specific polypeptide codes.

The following list is intended not to limit the invention but to provide guidance to programs and databases which are useful with the orthosomycin-specific nucleic acid codes, the everninomicin-specific nucleic acid codes, the avilamycin-specific nucleic acid codes, the orthosomycin-specific polypeptide code, the everninomicin-specific polypeptide code, and the avilamycin-specific polypeptide codes of the invention. The program and databases which may be used include, but are not limited to: MacPattern (EMBL), DiscoveryBase (Molecular Applications Group), GeneMine (Molecular Applications Group) Look (Molecular Applications Group), MacLook (Molecular Applications Group), BLAST and BLAST2 (NCBI), BLASTN and BLASTX (Altschul et al., J. Mol. Biol. 215:403 (1990)), FASTA (Person and Lipman, Proc. Nalt. Acad. Sci. USA, 85:2444 (1988)), FASTDB (Brutlag et al. Comp. App. Biosci. 6-237-245,1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II (Molecular Simulations Inc.), Discover (Molecular Simulations Inc.), CHARMm (Molecular Simulations Inc.), Felix (Molecular Simulations Inc.), DelPhi (Molecular Simulations Inc.), QuanteMM (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WetLab (Molecular Simulations Inc.), WetLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwents' World Drug Index database, the BioByteMasterFile database, the Genbank database, and the Gensyqn database. Many other programs and data bases would be apparent to one of skill in the art given the present disclosure.

Embodiments of the present invention include systems, particularly computer systems that store and manipulate the sequence information described herein. As used herein, “a computer system”, refers to the hardware components, software components, and data storage components used to analyze the reference sequences.

Preferably, the computer system is a general purpose system that comprises a processor and one or more internal data storage components for storing data, and one or more data retrieving devices for retrieving the data stored on the data storage components. A skilled artisan can readily appreciate that any one of the currently available computer systems are suitable.

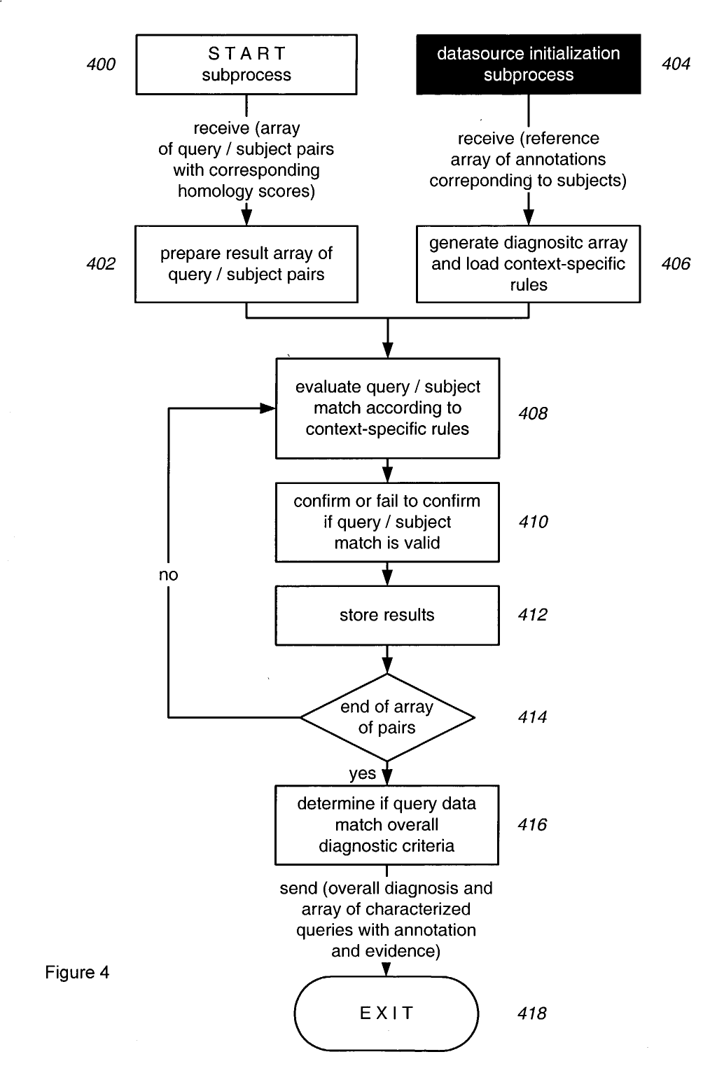

One example of a computer system is illustrated in FIG. 4. The computer system of FIG. 4 will includes a number of components connected to a central system bus 116, including a central processing unit 118 with internal 118 and external cache memory 120, system memory, 122, display adapter 102 connected to a monitor 100, network adapter 126 which may also be referred to as a network interface, internal modem 124, sound adapter 128, IO controller 132 to which may be connected a keyboard 140 and mouse 138, or other suitable input device such as a trackball or tablet, as well as external printer 134, and/or any number of external devices including but not limited to external modems, tape storage drives, or disk drives. One skilled in the art will readily appreciate that not all components illustrated in FIG. 4 are required to practice the invention and, likewise, additional components not illustrated in in FIG. 4 may be present in a computer system contemplates for use with the invention.

One or more host bus adapters 114 may be connected to the system bus 116. To host bus adapter 114 may optionally be connected one or more storage devices such as one or more disk drives 112 (removable or fixed), floppy drives 110, tape drives 108, digital versatile disk DVD drives 106, and compact disk CD ROM drives 104. The storage devices may operate in read-only mode and/or in read-write mode. Optical storage such as DVD 106 or CD Rom 104, are more commonly used in read-only mode, and fixed disk drives 112 are more likely to operate in read-write mode. Some computer systems may store large datasets that are larger that an individual disk drive 112, in which case specialized software can be used to allow data to span multiple disks. Examples of such software include but are not limited to Sun Microsystems' Solstice Disk Suite, or Sun Microsystems' RAID (redundant array of inexpensive disks) Manager. The computer system may be enclosed in an enclosure or case. The computer system may optionally include multiple central processing units 118, or multiple banks of memory 122.

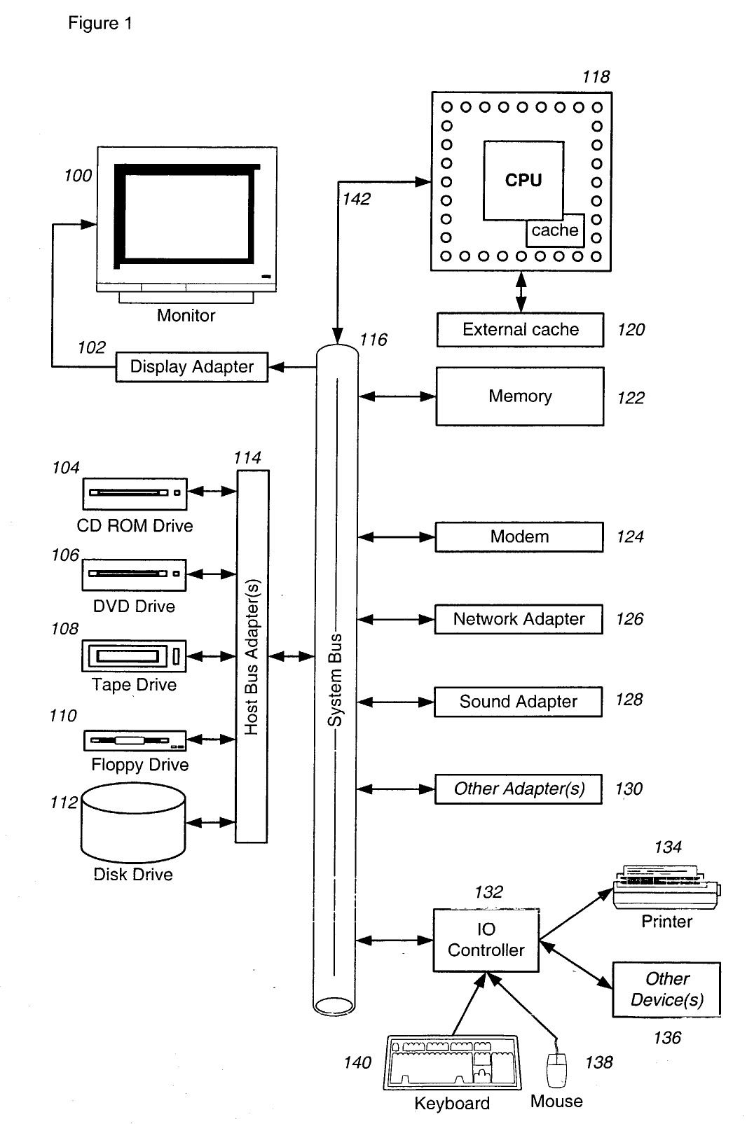

Arrows 142 in FIG. 1 indicate the interconnection of internal components of the computer system. The arrows are illustrative only and do not specify exact connection architecture. Some vendors may connect one or more central processing units to CPU/memory boards which then connect to the system bus.

Software for accessing and processing the reference sequences (such as sequence comparison software, analysis software as well as search tools, annotation tools, and modeling tools etc.) may reside in main memory 122 during execution.

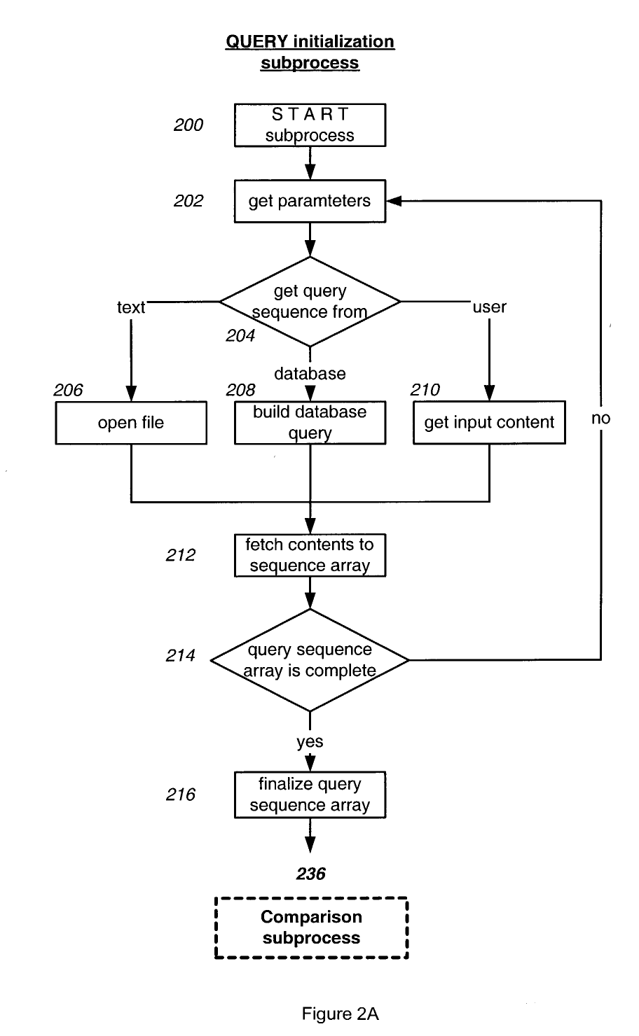

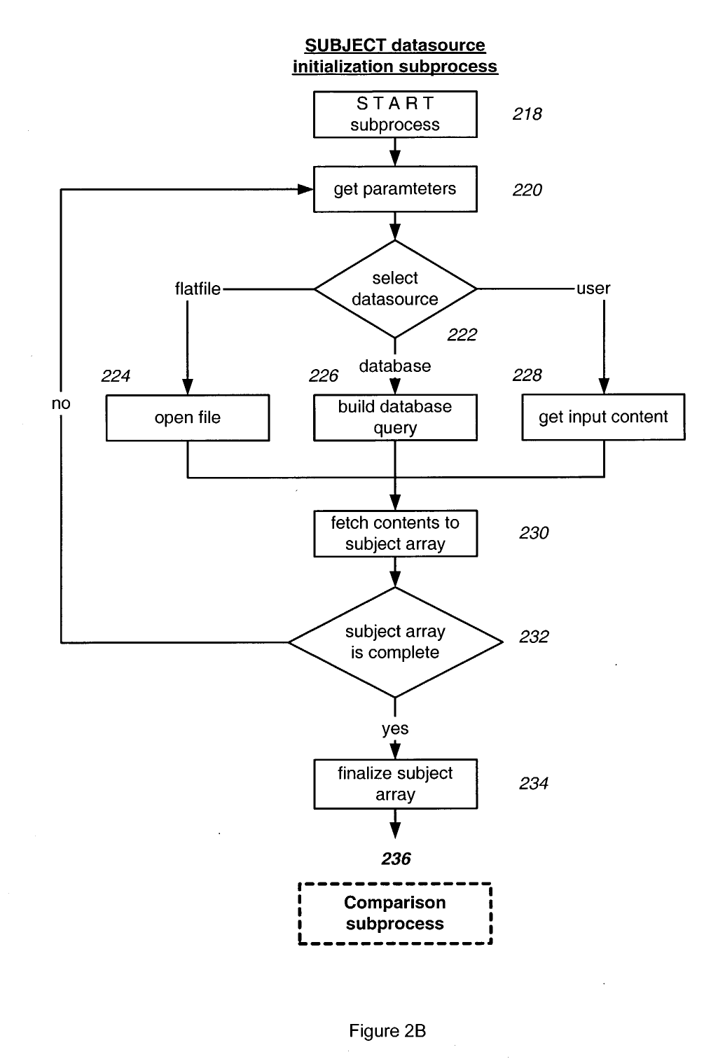

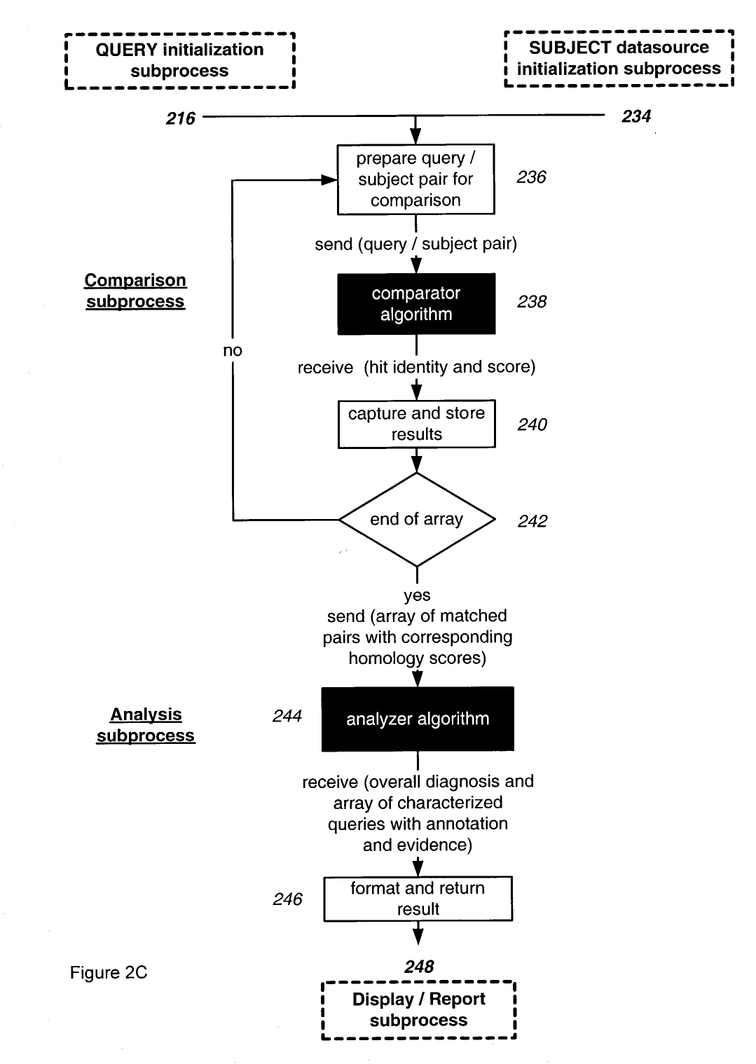

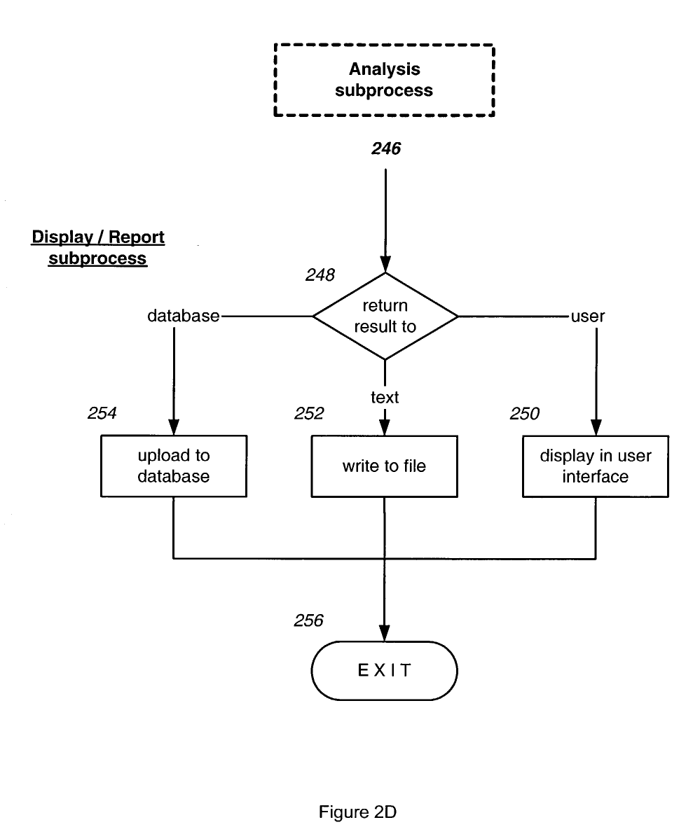

In a preferred embodiment, the computer system further comprises a sequence comparison software for comparing the nucleic acid codes of a query sequence stored on a computer readable medium to a subject sequence selected from an orthosomycin-specific nucleic acid code, an everninomicin-specific nucleic acid code, or an avilamycin-specific nucleic acid code which is also stored on a computer readable medium; or for comparing the polypeptide code of a query sequence stored on a computer readable medium to a subject sequence selected from an orthosomycin-specific polypeptide code, an everninomicin-specific polypeptide code, or an avilamycin-specific polypeptide code which is also stored on computer readable medium. A “sequence comparison software” refers to one or more programs that are implemented on the computer system to compare nucleotide sequences with other nucleotide sequences stored within the data storage means. The design of one example of a sequence comparison software is provided in FIG. 2.

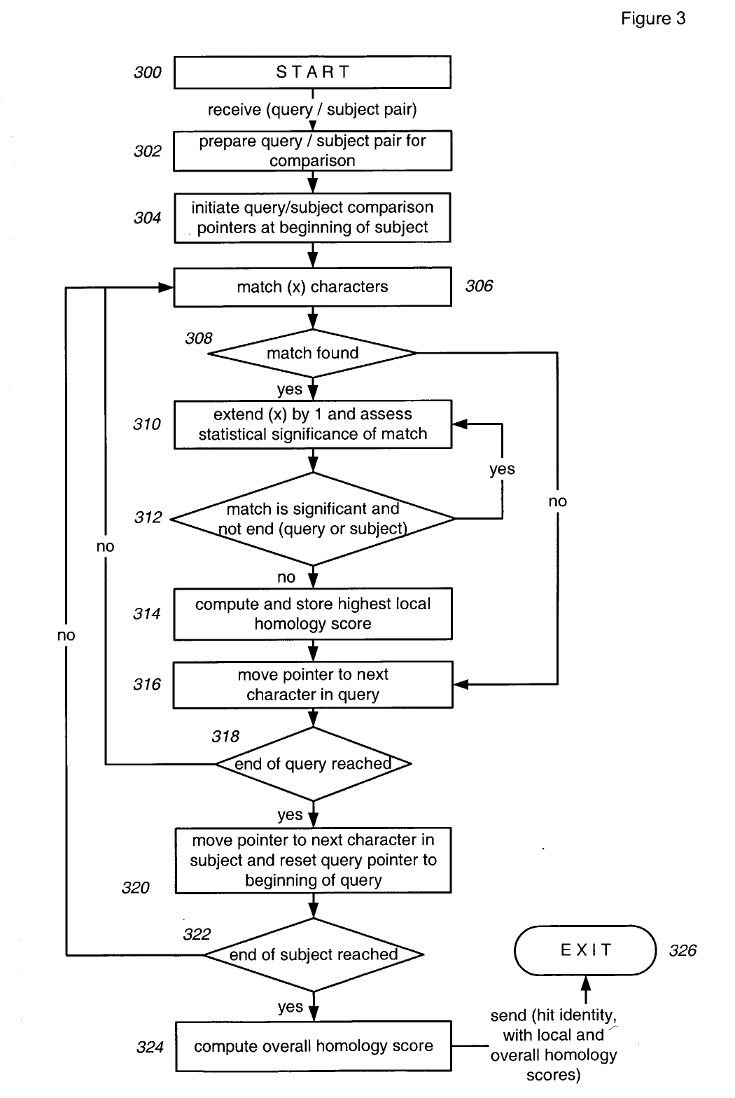

The sequence comparison software will typically employ one or more specialized comparator algorithms. Protein and/or nucleic acid sequence similarities may be evaluated using any of the variety of sequence comparator algorithms and programs known in the art. Such algorithms and programs include, but are no way limited to, TBLASTN, BLASTN, BLASTP, FASTA, TFASTA, CLUSTAL, HMMER, MAST, or other suitable algorithm known to those skilled in the art. (Pearson and Lipman, 1988, Proc. Natl. Acad. Sci USA 85(8):2444-2448; Altschul et al, 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J. Mol. Biol. 215(3):403-410; Altschul et al., 1993, Nature Genetics 3:266-272; Eddy S. R., Bioinformatics 14:755-763,1998; Bailey TL et al, J Steroid Biochem Mol Biol 1997 May; 62(1):29-44). One example of a comparator algorithm is illustrated in FIG. 3. Sequence comparator algorithms identified in this specification are particularly contemplated for use in this aspect of the invention.

The sequence comparison software will typically employ one or more specialized analyzer algorithms. One example of an analyzer algorithm is illustrated in FIG. 4. Any appropriate analyzer algorithm can be used to evaluate similarities, determined by the comparator algorithm, between query/subject pairs and based on context specific rules the annotation of a subject may be assigned to the query. A skilled artisan can readily determine the selection of an appropriate analyzer algorithm and appropriate context specific rules. Analyzer algorithms identified elsewhere in this specification are particularly contemplated for use in this aspect of the invention.

FIG. 2 is a flowchart of one example of a sequence comparison software for comparing query sequences to a subject sequence. The subject sequence may be selected from the reference sequences, in which case the software determines if a gene or set of genes represented by their nucleotide sequence, polypeptide sequence or other representation is significantly similar to the orthosomycin-specific nucleic acid codes, the everninomicin-specific nucleic acid codes, the avilamycin-specific nucleic acid codes, the orthosomycin-specific polypeptide codes, the everninomicin-specific polypeptide codes or the avilamycin-specific polypeptide codes of the invention. The software may be implemented in the C or C++ programming language, Java, Perl or other suitable programming language known to a person skilled in the art

Referring to FIG. 2, the query sequence(s) may be accessed by the program by means of input from the user 210, accessing a database 208 or opening a text file 206. The “query initialization process” allows a query sequence to be accessed and loaded into computer memory 122, or under control of the program stored on a disk drive 112 or other storage device in the form of a query sequence array 216. The query array 216 is one or more query nucleotide or polypeptide sequences accompanied by some appropriate identifiers. A dataset is accessed by the program by means of input from the user 228, accessing a database 226, or opening a text file 224. The “subject data source initialization process” of FIG. 2 refers to the method by which a reference dataset containing one or more sequences selected from the orthosomycin-specific nucleic acid code, the everninomicin-specific nucleic acid code, the avilamycin-specific nucleic acid code, the orthosomycin-specific polypeptide code, the everninomicin-specific polypeptide code, and the avilamycin-specific polypeptide code is loaded into computer memory 122, or under control of the program stored on a disk drive 112 or other storage device in the form of a subject array 234. The subject array 234 comprises one or more subject nucleotide or polypeptide sequences accompanied by some appropriate identifiers.

The “comparison subprocess” of FIG. 2 is the process by which the comparator algorithm 238 is invoked by the software for pairwise comparisons between query elements in the query sequence array 216, and subject elements in the subject array 234. The “comparator algorithm” of FIG. 2 refers to the pairwise comparisons between a query and subject pair from their respective arrays 216, 234. Comparator algorithm 238 may be any algorithm that acts on a query/subject pair, including but not limited to homology algorithms such as BLAST, Smith Waterman, Fasta, or statistical representation/probabilistic algorithms such as Markov models exemplified by HMMER, or other suitable algorithm known to one skilled in the art. Suitable algorithms would generally require a query/subject pair as input and return a score (an indication of likeness between the query and subject), usually through the use of appropriate statistical methods such as Karlin Altschul statistics used in BLAST, Forward or Viterbi algorithms used in Markov models, or other suitable statistics known to those skilled in the art.

The sequence comparison software of FIG. 2 also comprises a means of analysis of the results of the pairwise comparisons performed by the comparator algorithm 238. The “analysis subprocess” of FIG. 2 is a process by which the analyzer algorithm 244 is invoked by the software. The “analyzer algorithm” refers to a process by which annotation of a subject is assigned to the query based on query/subject similarity as determined by the comparator algorithm 238 according to context-specific rules coded into the program or dynamically loaded at runtime. Context-specific rules are what the program uses to determine if the annotation of the subject can be assigned to the query given the context of the comparison. These rules allow the software to qualify the overall meaning of the results of the comparator algorithm 238.

In one embodiment, context-specific rules may state that for a set of query sequences to be considered representative of an orthosomycin locus the comparator algorithm 238 must determine that the set of query sequences contain at least one query sequence that shows a statistical similarity to reference sequences corresponding to a nucleic acid sequence code for a polypeptide from two of the groups consisting of: (1) SEQ ID NO: 51; Genbank accession no. AAK83192; SEQ ID NO: 53; SEQ ID NO: 55; and polypeptides having at least 70% homology to a polypeptide having the sequence of SEQ ID NOS: 51, 53, 55 or Genbank accession no. AAK83192; (2) SEQ ID NO: 57; Genbank accession no. AAK83170; SEQ ID NO: 59; SEQ ID NO: 61; and polypeptides having at least 70% homology to a polypeptide having the sequence of SEQ ID NOS: 57, 59, 61 or Genbank accession no. AAK83170; (3) SEQ ID NO: 63, Genbank accession no. AAK83193, SEQ ID NO: 65, SEQ ID NO: 67, and polypeptides having at least 70% homology to a polypeptide having the sequence of SEQ ID NOS: 63, 65, 67 or Genbank accession no. AAK83193; (4) SEQ ID NO: 69, SEQ ID NO: 71, SEQ ID NO: 73, and polypeptides having at least 70% homology to a polypeptide having the sequence of SEQ ID NOS: 69, 71 or 73; (5) SEQ ID NO: 99, Genbank accession no. AAK83184, SEQ ID NO: 101, SEQ ID NO: 103, and polypeptides having at least 70% homology to a polypeptide having the sequence of SEQ ID NOS: 99, 101, 103 or Genbank accession no. AAK83184; (6) SEQ ID NO: 105, Genbank accession no. AAK83186, SEQ ID NO: 107, SEQ ID NO: 109, and polypeptides having at least 70% homology to a polypeptide having the sequence of SEQ ID NOS: 105, 107, 109 or Genbank accession no. AAK83186; (7) SEQ ID NO: 111, Genbank accession no. AAK83188, SEQ ID NO: 113, SEQ ID NO: 115, and polypeptides having at least 70% homology to a polypeptide having the sequence of SEQ ID NOS: 111, 113, 115 or Genbank accession no. AAK83188; (8) SEQ ID NO: 127, Genbank accession no. AAG32067, SEQ ID NO: 129, SEQ ID NO: 131 and polypeptides having at least 70% homology to a polypeptide having the sequence of SEQ ID NOS: 127, 129, 131 or Genbank accession no. AAG32067; (9) SEQ ID NO: 123, Genbank accession no. AAG32066, SEQ ID NO: 125 and polypeptides having at least 70% homology to a polypeptide having the sequence of SEQ ID NOS: 123, 125 or Genbank accession no. AAG32066; (10) SEQ ID NO: 153, Genbank accession no. AAK83187, SEQ ID NO: 155, SEQ ID NO: 157, and polypeptides having at least 70% homology to a polypeptide having the sequence of SEQ ID NOS: 153, 155, 157 or Genbank accession no. AAK83187; (11) SEQ ID NO: 159, SEQ ID NO: 161, SEQ ID NO: 163 and polypeptides having at least 70% homology to a polypeptide having the sequence of SEQ ID NOS: 159, 161 or 163; (12) SEQ ID NO: 167, SEQ ID NO: 173, Genbank accession no. AAK83181, SEQ ID NO: 169 and polypeptides having at least 70% homology to a polypeptide having the sequence of SEQ ID NOS: 167, 169, 173 or Genbank accession no. AAK83181; (13) SEQ ID NO: 175, SEQ ID NO: 177, SEQ ID NO: 179 and polypeptides having at least 70% homology to a polypeptide having the sequence of SEQ ID NOS: 175, 177 or 179; (14) SEQ ID NO: 165, SEQ ID NO: 171, SEQ ID NO: 169 and polypeptides having at least 70% homology to a polypeptide having the sequence of SEQ ID NOS: 165, 169 or 171; (15) SEQ ID NO: 193, Genbank accession no. AAK83189, SEQ ID NO: 195, SEQ ID NO: 197 and polypeptides having at least 70% homology to a polypeptide having the sequence of SEQ ID NOS: 193, 195, 197 or Genbank accession no. AAK83189; and (16) SEQ ID NO: 199, Genbank accession no. AAK83174, SEQ ID NO: 201 and polypeptides having at least 70% homology to a polypeptide having the sequence of SEQ ID NOS: 199, 201 or Genbank accession no. AAK83174; and (17) SEQ ID NO: 203, SEQ ID NO: 205, SEQ ID NO: 207 and polypeptides having at least 70% homology to a polypeptide having the sequence of SEQ ID NOS: 203, 205 or 207. Of course preferred context specific rules may specify a wide variety of thresholds for identifying orthosomycin biosynthetic gene or orthosomycin-producing organism without departing from the scope of the invention. Some preferred thresholds contemplates are that at least one query sequence in the set of query sequences show a statistical similarity to the nucleic acid code corresponding to 3 or 4 or 5 or 6 or 7 or 8 or 10 or more of the above 17 groups polypeptides diagnostic of othosomycin biosynthetic genes. Other preferred context specific rules set the level of homology required in each of the group may be set at 70%, 75%, 80%, 85%, 90%, 95% or 98% in regards to any one or more of the reference sequences.

In another embodiment context-specific rules may state that for a set of query sequences to be considered representative of an everninomicin-type orthosomycin, the comparator algorithm 238 must determine that at least one of the query sequences in the set of query sequences shows a statistical similarity to reference sequences corresponding to a nucleic acid sequence code for a polypeptide from one of the groups consisting of: (1) SEQ ID NO: 209, SEQ ID NO: 211 and polypeptides having at least 70% homology to a polypeptide of SEQ ID NO: 209 or SEQ ID NO: 211; (2) SEQ ID NO: 213, SEQ ID NO: 215 and polypeptides having at least 70% homology to a polypeptide of SEQ ID NO: 213 or SEQ ID NO: 215; (3) SEQ ID NO: 217, SEQ ID NO: 219 and polypeptides having at least 70% homology to a polypeptide of SEQ ID NO: 217 or SEQ ID NO: 219; (4) SEQ ID NO: 221, SEQ ID NO: 223 and polypeptides having at least 70% homology to a polypeptide of SEQ ID NO: 221 or SEQ ID NO: 223; (5) SEQ ID NO: 225, SEQ ID NO: 227 and polypeptides having at least 70% homology to a polypeptide of SEQ ID NO: 225 or SEQ ID NO: 227; (6) SEQ ID NO: 229, SEQ ID NO: 231 and polypeptides having at least 70% homology to a polypeptide of SEQ ID NO: 229 or SEQ ID NO: 231; (7) SEQ ID NO: 233, SEQ ID NO: 235 and polypeptides having at least 70% homology to a polypeptide of SEQ ID NO: 233 or SEQ ID NO: 235; (8) SEQ ID NO: 237, SEQ ID NO: 239 and polypeptides having at least 70% homology to a polypeptide of SEQ ID NO: 237 or SEQ ID NO: 239; and (9) SEQ ID NO: 241, SEQ ID NO: 243 and polypeptides having at least 70% homology to a polypeptide of SEQ ID NO: 241 or SEQ ID NO: 243. Of course preferred context specific rules may specify a wide variety of thresholds for identifying everninomicin-type orthosomycin biosynthetic genes or everninomicin-type orthosomycin-producing organism without departing from the scope of the invention. Some preferred thresholds contemplates are that at least one query sequence in the set of query sequences show a statistical similarity to the nucleic acid code corresponding to 2 or 3 or 4 or 5 or 6 or 7 or 8 or 9 the above 9 groups polypeptides diagnostic of everninomicin-type othosomycin biosynthetic genes. In a highly preferred embodiment, the set of query sequences would contain at least one query sequence showing a statistical similarity to the nucleic acid code corresponding to 2 or 3 or 4 or 5 or 6 or 7 or 8 or 9 of the 9 groups polypeptides diagnostic of everninomicin biosynthetic gene cluster, together with at least one query sequence in the set of query sequences showing a statistical similarity to the nucleic acid code corresponding to 3 or 4 or 5 or 6 or 7 or 8 or 10 or more of the above 17 groups of polypeptides diagnostic of othosomycin biosynthetic genes. Other preferred context specific rules set level of homology required in each of the group may be at 70%, 75%, 80%, 85%, 90%, 95% or 98% in regards to any one or more of the reference sequences.

In another embodiment context-specific rules may state that for a set of query sequences to be considered representative of an avilamycin-type orthosomycin locus the comparator algorithm 238 must determine that the set of query sequences contain at least one query sequence that shows a statistical similarity to reference sequences corresponding to a nucleic acid sequence code for a polypeptide from one of the groups consisting of (1) SEQ ID NO: 245, Genbank accession no. AAG32068 and polypeptides having at least 70% homology to a polypeptide of SEQ ID NO: 245 or Genbank accession no. AAG32068; (2) SEQ ID NO: 247, Genbank accession no. AAK83183, and polypeptides having at least 70% homology to a polypeptide of SEQ ID NO: 247 or Genbank accession no. AAK83183; (3) SEQ ID NO: 249, accession no. AAG32069, and polypeptides having at least 70% homology to a polypeptide of SEQ ID NO: 249 or Genbank accession no. AAG32069; (4) SEQ ID NO: 251, Genbank accession no. AAK83172, and polypeptides having at least 70% homology to a polypeptide of SEQ ID NO: 251 or Genbank accession no. AAK83172; (5) SEQ ID NO: 253, Genbank accession no. AAK83171 and polypeptides having at least 70% homology to a polypeptide of SEQ ID NO: 253 or Genbank accession no. AAK83171; (6) SEQ ID NO: 255, Genbank accession no. AAK83175, and polypeptides having at least 70% homology to a polypeptide of SEQ ID NO: 255 or Genbank accession no. AAK83175. Of course preferred context specific rules may specify a wide variety of thresholds for identifying an avilamycin-type orthosomycin biosynthetic gene or an avilamycin-type orthosomycin-producing organism without departing from the scope of the invention. Some preferred thresholds contemplates are that at least one query sequence in the set of query sequences show a statistical similarity to the nucleic acid code corresponding to 2, 3 or 4 or 5 or 6 of the above groups polypeptides diagnostic of avilamycin-type othosomycin biosynthetic genes. In a highly preferred embodiment, the set of query sequences would contain at least one query sequence showing a statistical similarity to the nucleic acid code corresponding to 2 or 3 or 4 or 5 or 6 groups polypeptides diagnostic of avilamycin-type biosynthetic gene cluster, together with at least one query sequence in the set of query sequences showing a statistical similarity to the nucleic acid code corresponding to 3 or 4 or 5 or 6 or 7 or 8 or 10 or more of the above 17 groups of polypeptides diagnostic of othosomycin biosynthetic genes. Other preferred context specific rules set the level of homology required in each of the group may at 70%, 75%, 80%, 85%, 90%, 95% or 98% in regards to any one or more of the reference sequences.

Thus, the analysis subprocess may be employed in conjunction with any other context specific rules and may be adapted to suit different embodiments. The principal function of the analyzer algorithm 244 is to assign meaning or a diagnosis to a query or set of queries based on context specific rules that are application specific and may be changed without altering the overall role of the analyzer algorithm 244.

Finally the sequence comparison software of FIG. 2 comprises a means of returning of the results of the comparisons by the comparator algorithm 238 and analyzed by the analyzer algorithm 244 to the user or process that requested the comparison or comparisons. The “display/report subprocess” of FIG. 2 is the process by which the results of the comparisons by the comparator algorithm 238 and analyses by the analyzer algorithm 244 are returned to the user or process that requested the comparison or comparisons. The results 240, 246 may be written to a file 252, displayed in some user interface such as a console, custom graphical interface, web interface, or other suitable implementation specific interface, or uploaded to some database such as a relational database, or other suitable implementation specific database.

Once the results have been returned to the user or process that requested the comparison or comparisons the program exits.

The principle of the sequence comparison software of FIG. 2 is to receive or load a query or queries, receive or load a reference dataset, then run a pairwise comparison by means of the comparator algorithm 238, then evaluate the results using an analyzer algorithm 244 to arrive at a determination if the query or queries bear significant similarity to the reference sequences, and finally return the results to the user or calling program or process.

FIG. 3 is a flow diagram illustrating one embodiment of a comparator algorithm 238 process in a computer for determining whether two sequences are homologous. The comparator algorithm receives a query/subject pair for comparison, performs an appropriate comparison, and returns the pair along with a calculated degree of similarity.

Referring to FIG. 3, the comparison is initiated at the beginning of sequences 304. A match of (x) characters is attempted 306 where (x) is a user specified number. If a match is not found the query sequence is advanced 316 by one polypeptide with respect to the subject, and if the end of the query has not been reached 318 another match of (x) characters is attempted 306. Thus if no match has been found the query is incrementally advanced in entirety past the initial position of the subject, once the end of the query is reached 318, the subject pointer is advanced by 1 polypeptide and the query pointer is set to the beginning of the query 318. If the end of the subject has been reached and still no matches have been found a null homology result score is assigned 324 and the algorithm returns the pair of sequences along with a null score to the calling process or program. The algorithm then exits 326. If instead a match is found 308, an extension of the matched region is attempted 310 and the match is analyzed statistically 312. The extension may be unidirectional or bidirectional. The algorithm continues in a loop extending the matched region and computing the homology score, giving penalties for mismatches taking into consideration that given the chemical properties of the polypeptide side chains not all mismatches are equal. For example a mismatch of a lysine with an arginine both of which have basic side chains receive a lesser penalty than a mismatch between lysine and glutamate which has an acidic side chain. The extension loop stops once the accumulated penalty exceeds some user specified value, or of the end of either sequence is reached 312. The maximal score is stored 314, and the query sequence is advanced 316 by one polypeptide with respect to the subject, and if the end of the query has not been reached 318 another match of (x) characters is attempted 306. The process continues until the entire length of the subject has been evaluated for matches to the entire length of the query. All individual scores and alignments are stored 314 by the algorithm and an overall score is computed 324 and stored. The algorithm returns the pair of sequences along with local and global scores to the calling process or program. The algorithm then exits 326.

Comparator algorithm 238 algorithm may be represented in pseudocode as follows:

| | |
|---|---|
| INPUT: | Q[m]: query, m is the length |
| | S[n]: subject, n is the length |

| | |
|---|---|
| x: | x is the size of a segment |

START:
for each i in [1,n] do

for each j in [1,m] do

if ( j + x − 1 ) <= m and ( i + x −1 ) <= n then

if Q(j, j+x−1) = S(i, i+x−1) then

k=1;
while Q(j, j+x−1+k ) = S(i, i+x−1+ k) do

k++;

Store highest local homology

Compute overall homology score
Return local and overall homology scores
END.

The comparator algorithm 238 may be written for use on nucleotide sequences, in which case the scoring scheme would be implemented so as to calculate scores and apply penalties based on the chemical nature of nucleotides. The comparator algorithm 238 may also provide for the presence of gaps in the scoring method for nucleotide or polypeptide sequences.

BLAST is one implementation of the comparator algorithm 238. HMMER is another implementation of the comparator algorithm 238 based on Markov model analysis. In a HMMER implementation a query sequence would be compared to a mathematical model representative of a subject sequence or sequences rather than using sequence homology.

FIG. 4 is a flow diagram illustrating an analyzer algorithm 244 process for detecting the presence of an orthosomycin biosynthetic locus, an everninomicin-type orthosomycin biosynthetic locus or an avilamycin-type orthosomycin biosynthetic locus. The analyzer algorithm of FIG. 4 may be used in the process by which the annotation of a subject is assigned to the query based on their similarity as determined by the comparator algorithm 238 and according to context-specific rules coded into the program or dynamically loaded at runtime. Context sensitive rules are what determines if the annotation of the subject can be assigned to the query given the context of the comparison. Context specific rules set the thresholds for determining the level and quality of similarity that would be accepted in the process of evaluating matched pairs.

The analyzer algorithm 244 receives as its input an array of pairs that had been matched by the comparator algorithm 238. The array consists of at least a query identifier, a subject identifier and the associated value of the measure of their similarity. To determine if a group of query sequences includes an sequences diagnostic of an avilamycin-type orthosomycin biosynthetic gene cluster, a reference or diagnostic array 406 is generated by accessing a data source and retrieving avilamycin specific information 404 relating to avilamycin-specific nucleic acid codes and avilamycin-specific polypeptide codes. Diagnostic array 406 consists at least of subject identifiers and their associated annotation. Annotation may include reference to the nine protein families diagnostic of avilamycin-type biosynthetic genes clusters, i.e. ABCD, DEPN, MEMD, REBU, UNAI and UNBR. Annotation may also include information regarding exclusive presence in loci of a specific structural class or may include previously computed matches to other databases, for example databases of motifs. Once the algorithm has successfully generated or received the two necessary arrays 402, 406, and holds in memory any context specific rules, each matched pair as determined by the comparator algorithm 238 can be evaluated. The algorithm will perform an evaluation 408 of each matched pair and based on the context specific rules confirm or fail to confirm the match as valid 410. In cases of successful confirmation of the match 410 the annotation of the subject is assigned to the query. Results of each comparison are stored 412. The loop ends when the end of the query/subject array is reached. Once all query/subject pairs have been evaluated against avilamycin-specific nucleic acid codes and avilamycin-specific polypeptide codes, a final determination can be made if the query set of ORFs represents an aviiamycin locus 416.

The algorithm then returns the overall diagnosis and an array of characterized query/subject pairs along with supporting evidence to the calling program or process and then terminates 418.

The analyzer algorithm 244 may be configured to dynamically load different diagnostic arrays and context specific rules. It may be used for example in the comparison of query/subject pairs with diagnostic subjects for other biosynthetic pathways, such as everninomicin-specific nucleic acid codes or everninomicin-specific polypeptide codes, or other sets of annotated subjects.

The present invention will be further described with reference to the following examples; however, it is to be understood that the present invention is not limited to such examples.

### EXAMPLE 1

### Identification of the Everninomicin Biosynthetic Locus in Micromonospora carbonacea var. aurantiaca

The microorganism Micromonospora carbonacea var. aurantiaca NRRL 2997 was obtained from the Agriculture Research Service Culture Collection of the United States Department of Agriculture, 1815 N. University Street, Peoria, Ill. 61604. The everninomicin compound produced by strain NRRL 2997 is described in U.S. Pat. No. 3,499,078. The biosynthetic locus for everninomicin was identified from strain NRRL 2997 (EVER) according to the method described in Canadian patent application CA 2,352,451. The sequences obtained from cosmids containing overlapping genomic inserts spanning the biosynthetic locus for everninomicin were identified. Within the sequences of the cosmid inserts, numerous ORFs encoding polypeptides having homology to known proteins were identified. Homology was determined using the program BLASTP version 2.2.2 with the default parameters. Contiguous nucleotide sequences and deduced amino acid sequences of EVER are provided. EVER is formed of three contiguous DNA sequences (SEQ ID NOS: 280, 281 and 282) which are arranged such that, as found within the EVER, the 3′ end of DNA contig 1 (SEQ ID NO: 280) is adjacent to the 5′ end of DNA contig 2 (SEQ ID NO: 281), which in turn is adjacent to the 5′ end of DNA contig 3 SEQ ID NO: 282). The ORFs present in EVER encode 50 polypeptides, the sequences of which are provided as follows: The amino acid sequence of ORF 1 (SEQ ID NO 263) is deduced from the nucleic acid sequence of SEQ ID NO 264 drawn from contig 1 (SEQ ID NO 280). The amino acid sequence of ORF 2 (SEQ ID NO 89) is deduced from the nucleic acid sequence of SEQ ID NO 90 drawn from contig 1 (SEQ ID NO 280). The amino acid sequence of ORF 3 (SEQ ID NO 225) is deduced from the nucleic acid sequence of SEQ ID NO 226 drawn from contig 1 (SEQ ID NO 280). The amino acid sequence of ORF 4 (SEQ ID NO 237) is deduced from the nucleic acid sequence of SEQ ID NO 238 drawn from contig 1 (SEQ ID NO 280). The amino acid sequence of ORF 5 (SEQ ID NO 113) is deduced from the nucleic acid sequence of SEQ ID NO 114 drawn from contig 1 (SEQ ID NO 280). The amino acid sequence of ORF 6 (SEQ ID NO 119) is deduced from the nucleic acid sequence of SEQ ID NO 120 drawn from contig 1 (SEQ ID NO 280). The amino acid sequence of ORF 7 (SEQ ID NO 49) is deduced from the nucleic acid sequence of SEQ ID NO 50 drawn from contig 1 (SEQ ID NO 280). The amino acid sequence of ORF 8 (SEQ ID NO 65) is deduced from the nucleic acid sequence of SEQ ID NO 66 drawn from contig 1 (SEQ ID NO 280). The amino acid sequence of ORF 9 (SEQ ID NO 201) is deduced from the nucleic acid sequence of SEQ ID NO 202 drawn from contig 1 (SEQ ID NO 280). The amino acid sequence of ORF 10 (SEQ ID NO 15) is deduced from the nucleic acid sequence of SEQ ID NO 16 drawn from contig 1 (SEQ ID NO 280). The amino acid sequence of ORF 11 (SEQ ID NO 95) is deduced from the nucleic acid sequence of SEQ ID NO 96 drawn from contig 1 (SEQ ID NO 280). The amino acid sequence of ORF 12 (SEQ ID NO 71) is deduced from the nucleic acid sequence of SEQ ID NO 72 drawn from contig 1 (SEQ ID NO 280). The amino acid sequence of ORF 13 (SEQ ID NO 125) is deduced from the nucleic acid sequence of SEQ ID NO 126 drawn from contig 1 (SEQ ID NO 280). The amino acid sequence of ORF 14 (SEQ ID NO 83) is deduced from the nucleic acid sequence of SEQ ID NO 84 drawn from contig 1 (SEQ ID NO 280). The amino acid 'sequence of ORF 15 (SEQ ID NO 101) is deduced from the nucleic acid sequence of SEQ ID NO 102 drawn from contig 1 (SEQ ID NO 280). The amino acid sequence of ORF 16 (SEQ ID NO 47) is deduced from the nucleic acid sequence of SEQ ID NO 48 drawn from contig 1 (SEQ ID NO 280). The amino acid sequence of ORF 17 (SEQ ID NO 195) is deduced from the nucleic acid sequence of SEQ ID NO 196 drawn from contig 1 (SEQ ID NO 280). The amino acid sequence of ORF 18 (SEQ ID NO 155) is deduced from the nucleic acid sequence of SEQ ID NO 156 drawn from contig 1 (SEQ ID NO 280). The amino acid sequence of ORF 19 (SEQ ID NO 107) is deduced from the nucleic acid sequence of SEQ ID NO 108 drawn from contig 1 (SEQ ID NO 280). The amino acid sequence of ORF 20 (SEQ ID NO 77) is deduced from the nucleic acid sequence of SEQ ID NO 78 drawn from contig 1 (SEQ ID NO 280). The amino acid sequence of ORF 21 (SEQ ID NO 221) is deduced from the nucleic acid sequence of SEQ ID NO 222 drawn from contig 1 (SEQ ID NO 280). The amino acid sequence of ORF 22 (SEQ ID NO 151) is deduced from the nucleic acid sequence of SEQ ID NO 152 drawn from contig 1 (SEQ ID NO 280). The amino acid sequence of ORF 23 (SEQ ID NO 143) is deduced from the nucleic acid sequence of SEQ ID NO 144 drawn from contig 1 (SEQ ID NO 280). The amino acid sequence of ORF 24 (SEQ ID NO 53) is deduced from the nucleic acid sequence of SEQ ID NO 54 drawn from contig 1 (SEQ ID NO 280). The amino acid sequence of ORF 25 (SEQ ID NO 205) is deduced from the nucleic acid sequence of SEQ ID NO 206 drawn from contig 1 (SEQ ID NO 280). The amino acid sequence of ORF 26 (SEQ ID NO 161) is deduced from the nucleic acid sequence of SEQ ID NO 162 drawn from contig 1 (SEQ ID NO 280). The amino acid sequence of ORF 27 (SEQ ID NO 257) is deduced from the nucleic acid sequence of SEQ ID NO 258 drawn from contig 1 (SEQ ID NO 280). The amino acid sequence of ORF 28 (SEQ ID NO 135) is deduced from the nucleic acid sequence of SEQ ID NO 136 drawn from contig 1 (SEQ ID NO 280). The amino acid sequence of ORF 29 (SEQ ID NO 3) is deduced from the nucleic acid sequence of SEQ ID NO 4 drawn from contig 1 (SEQ ID NO 280). The amino acid sequence of ORF 30 (SEQ ID NO 35) is deduced from the nucleic acid sequence of SEQ ID NO 36 drawn from contig 1 (SEQ ID NO 280). The amino acid sequence of ORF 31 (SEQ ID NO 169) is deduced from the nucleic acid sequence of SEQ ID NO 170 drawn from contig 1 (SEQ ID NO 280). The amino acid sequence of ORF 32 (SEQ ID NO 183) is deduced from the nucleic acid sequence of SEQ ID NO 184 drawn from contig 1 (SEQ ID NO 280). The amino acid sequence of ORF 33 (SEQ ID NO 177) is deduced from the nucleic acid sequence of SEQ ID NO 178 drawn from contig 1 (SEQ ID NO 280). The amino acid sequence of ORF 34 (SEQ ID NO 29) is deduced from the nucleic acid sequence of SEQ ID NO 30 drawn from contig 1 (SEQ ID NO 280). The amino acid sequence of ORF 35 (SEQ ID NO 59) is deduced from the nucleic acid sequence of SEQ ID NO 60 drawn from contig 1 (SEQ ID NO 280). The amino acid sequence of ORF 36 (SEQ ID NO 189) is deduced from the nucleic acid sequence of SEQ ID NO 190 drawn from contig 1 (SEQ ID NO 280). The amino acid sequence of ORF 37 (SEQ ID NO 141) is deduced from the nucleic acid sequence of SEQ ID NO 142 drawn from contig 1 (SEQ ID NO 280). The amino acid sequence of ORF 38 (SEQ ID NO 41) is deduced from the nucleic acid sequence of SEQ ID NO 42 drawn from contig 1 (SEQ ID NO 280). The amino acid sequence of ORF 39 (SEQ ID NO 9) is deduced from the nucleic acid sequence of SEQ ID NO 10 drawn from contig 1 (SEQ ID NO 280). The amino acid sequence of ORF 40 (SEQ ID NO 129) is deduced from the nucleic acid sequence of SEQ ID NO 130 drawn from contig 1 (SEQ ID NO 280). As indicated in Table II-B, the sequence of ORF 41 provided herein contains a gap. The amino acid sequence of ORF 41, C-terminus (SEQ ID NO 23) is deduced from the nucleic acid sequence of SEQ ID NO 24 drawn from contig 1 (SEQ ID NO 280). The amino acid sequence of ORF 41, N-terminus (SEQ ID NO 21) is deduced from the nucleic acid sequence of SEQ ID NO 22 drawn from contig 2 (SEQ ID NO 281). The amino acid sequence of ORF 42, C-terminus only (SEQ ID NO 233) is deduced from the nucleic acid sequence of SEQ ID NO 234 drawn from contig 3 (SEQ ID NO 282). The amino acid sequence of ORF 43 (SEQ ID NO 209) is deduced from the nucleic acid sequence of SEQ ID NO 210 drawn from contig 3 (SEQ ID NO 282). The amino acid sequence of ORF 44 (SEQ ID NO 229) is deduced from the nucleic acid sequence of SEQ ID NO 230 drawn from contig 3 (SEQ ID NO 282). The amino acid sequence of ORF 45 (SEQ ID NO 217) is deduced from the nucleic acid sequence of SEQ ID NO 218 drawn from contig 3 (SEQ ID NO 282). The amino acid sequence of ORF 46 (SEQ ID NO 213) is deduced from the nucleic acid sequence of SEQ ID NO 214 drawn from contig 3 (SEQ ID NO 282). The amino acid sequence of ORF 47 (SEQ ID NO 241) is deduced from the nucleic acid sequence of SEQ ID NO 242 drawn from contig 3 (SEQ ID NO 282). The amino acid sequence of ORF 48 (SEQ ID NO 259) is deduced from the nucleic acid sequence of SEQ ID NO 260 drawn from contig 3 (SEQ ID NO 282). The amino acid sequence of ORF 49 (SEQ ID NO 267) is deduced from the nucleic acid sequence of SEQ ID NO 268 drawn from contig 3 (SEQ ID NO 282). The amino acid sequence of ORF 50 (SEQ ID NO 261) is deduced from the nucleic acid sequence of SEQ ID NO 262 drawn from contig 3 (SEQ ID NO 282).

The ORFs in EVER have been assigned a putative function and protein family designation based on homology to known proteins as indicated in Table II-A. The position, length and orientation of each EVER ORF within SEQ ID NOS: 280, 281 and 282 is provided in Table II-B.

TABLE II-A
EVER blast table

| ORF | Family | #aa | GenBank homology | probability | % identity | % similarity | proposed function of GenBank match |
|---|---|---|---|---|---|---|---|
| 1 | MTBA | 250 | AAG23269.1, 250aa | 5e−89 | 150/249 (60.24%) | 188/249 (75.5%) | probable O-methyltransferase, |
| | | | | | | | Saccharopolyspora spinosa |
| | | | CAC32469.1, 261aa | 4e−83 | 147/249 (59.06%) | 185/249 (74.3%) | O-methyltransferase, Streptomyces olivaceus |
| | | | AAD41819.1, 256aa | 8e−82 | 139/250 (55.6%) | 181/250 (72.4%) | macrocin-O-methyltransferase, |
| | | | | | | | Streptomyces fradiae |
| 2 | MEMK | 442 | CAC48373.1, 452aa | 2e−84 | 186/439 (42.37%) | 239/439 (54.44%) | putative antiporter, |
| | | | | | | | Amycolatopsis mediterranei |
| | | | NP_103653.1, 737aa | 1e−38 | 96/397 (24.18%) | 185/397 (46.6%) | Na/H antiporter, Mesorhizobium loti |
| | | | NP_440311.1, 410aa | 4e−25 | 72/270 (26.67%) | 130/270 (48.15%) | Na/H antiporter, Synechocystis sp. |
| 3 | MTFG | 362 | AAL02176.1, 341aa | 9e−06 | 65/292 (22.26%) | 111/292 (38.01%) | rRNA methyltransferase, Enterococcus faecium |
| 4 | OXCO | 489 | AAK61713.1, 475aa | 2e−99 | 198/460 (43.04%) | 263/460 (57.17%) | oxidoreductase-like protein, Streptomyces |
| | | | | | | | aureofaciens |
| | | | AAL82808.1, 431aa | 3e−86 | 178/381 (46.72%) | 225/381 (59.06%) | JadZ, Streptomyces venezuelae |
| | | | Q12737, 572aa | 2e−65 | 173/501 (34.53%) | 250/501 (49.9%) | Bilirubin oxidase precursor, Myrothecium |
| | | | | | | | verrucaria |
| 5 | MTFF | 274 | AAK83188.1, 277aa | 2e−81 | 155/270 (57.41%) | 176/270 (65.19%) | AviG3, Streptomyces viridochromogenes |
| 6 | MTFV | 429 | AAK83176.1, 407aa | 2e−78 | 180/406 (44.33%) | 232/406 (57.14%) | methyltransferase, Streptomyces |
| | | | | | | | viridochromogenes |
| | | | AAD41823.1, 418aa | 7e−78 | 179/407 (43.98%) | 228/407 (56.02%) | NDP-hexose 3-C-methyltransferase, |
| | | | | | | | Streptomyces fradiae |
| 7 | MTFH | 394 | AAD12164.1, 395aa | 1e−77 | 157/342 (45.91%) | 205/342 (59.94%) | methyltransferase, |
| | | | | | | | Streptomyces fradiae |
| | | | AAG23270.1, 395aa | 3e−75 | 148/333 (44.44%) | 197/333 (59.16%) | probable O-methyltransferase, |
| | | | | | | | Saccharopolyspora spinosa |
| | | | CAC32466.1, 378aa | 4e−72 | 156/314 (49.68%) | 185/314 (58.92%) | O-methyltransferase, Streptomyces olivaceus |
| 8 | GTFH | 339 | AAK83193.1, 342aa | 6e−92 | 174/342 (50.88%) | 221/342 (64.62%) | putative glycosyltransferase, |
| | | | | | | | Streptomyces viridochromogenes |
| | | | NP_102448.1, 353aa | 2e−09 | 80/313 (25.56%) | 122/313 (38.98%) | probable sugar transferase, |
| | | | | | | | Mesorhizobium loti |
| 9 | UEVB | 137 | AAK83174.1, 136aa | 6e−62 | 102/133 (76.69%) | 122/133 (91.73%) | AviX10, Streptomyces viridochromogenes |
| 10 | DEPG | 314 | T46669, 328aa | 1e−38 | 113/309 (36.57%) | 142/309 (45.95%) | probable dTDP-4-keto-6-deoxyhexose |
| | | | | | | | reductase, Streptomyces nogalater |
| | | | AAB63047.1, 307aa | 2e−32 | 99/305 (32.46%) | 137/305 (44.92%) | thymidine diphospho-4-keto-2,3,6- |
| | | | | | | | trideoxyhexulose reductase, Streptomyces |
| | | | | | | | peucetius |
| | | | AAF72549.1, 346aa | 7e−31 | 107/318 (33.65%) | 135/318 (42.45%) | NDP-hexose 4-ketoreductase, |
| | | | | | | | Streptomyces fradiae |
| 11 | MTFA | 342 | P42712, 376aa | 1e−58 | 132/333 (39.64%) | 176/333 (52.85%) | O-demethylpuromycin-O-methyltransferase, |
| | | | | | | | Streptomyces alboniger |
| | | | BAA32132.1, 305aa | 2e−55 | 123/301 (40.86%) | 152/301 (50.5%) | Orf4, Streptomyces griseus |
| | | | CAB76315.1, 342aa | 3e−31 | 96/322 (29.81%) | 149/322 (46.27%) | putative O-methyltransferase., |
| | | | | | | | Streptomyces coelicolor |
| 12 | HOXG | 276 | | | | | No homolog by blastp in GenBank nr |
| | | | | | | | protein database |
| 13 | MTIA | 265 | AAG32066.1, 287aa | 2e−73 | 145/266 (54.51%) | 188/266 (70.68%) | rRNA methyltransferase, Streptomyces |
| | | | | | | | viridochromogenes |
| | | | NP_294741.1, 265aa | 3e−27 | 89/247 (36.03%) | 128/247 (51.82%) | rRNA methylase, Deinococcus radiodurans |
| | | | NP_104368.1, 274aa | 1e−26 | 83/248 (33.47%) | 128/248 (51.61%) | rRNA methylase, Mesorhizobium loti |
| 14 | KASA | 344 | AAK83178.1, 343aa | 1e−127 | 226/345 (65.51%) | 265/345 (76.81%) | AviN, Streptomyces viridochromogenes |
| | | | AAG29787.1, 355aa | 3e−77 | 153/346 (44.22%) | 207/346 (59.83%) | 3-ketoacyl-ACP-synthase, |
| | | | | | | | Streptomyces rishiriensis |
| | | | CAB71914.1, 346aa | 9e−69 | 139/343 (40.52%) | 200/343 (58.31%) | hypothetical protein, Streptomyces coelicolor |
| 15 | MTFD | 240 | AAK83184.1, 240aa | 4e−72 | 135/235 (57.45%) | 168/235 (71.49%) | putative methyltransferase, |
| | | | | | | | Streptomyces viridochromogenes |
| | | | CAA70016.1, 253aa | 3e−04 | 40/119 (33.61%) | 50/119 (42.02%) | methyltransferase, Streptomyces griseus |
| 16 | GTFA | 438 | AAK83182.1, 430aa | 1e−167 | 280/428 (65.42%) | 336/428 (78.5%) | putative glycosyltransferase, |
| | | | | | | | Streptomyces viridochromogenes |
| | | | T46681, 411aa | 4e−59 | 146/416 (35.1%) | 213/416 (51.2%) | probable glycosyltransferase, |
| | | | | | | | Streptomyces nogalater |
| | | | AAC64928.1, 393aa | 1e−43 | 132/405 (32.59%) | 184/405 (45.43%) | glycosyltransferase, Streptomyces argillaceus |
| 17 | UEVA | 405 | AAK83189.1, 396aa | 1e−164 | 271/391 (69.31%) | 306/391 (78.26%) | AviX12, Streptomyces viridochromogenes |
| 18 | OXRV | 313 | AAK83187.1, 314aa | 1e−132 | 221/301 (73.42%) | 247/301 (82.06%) | putative oxygenase, Streptomyces |
| | | | | | | | viridochromogenes |
| | | | AAK83181.1, 223aa | 1e−08 | 60/218 (27.52%) | 89/218 (40.83%) | putative oxygenase, Streptomyces |
| | | | | | | | viridochromogenes |
| | | | NP_521770.1, 263aa | 9e−07 | 66/228 (28.95%) | 89/228 (39.04%) | probable hydroxylase, Ralstonia solanacearum |
| 19 | MTFE | 243 | AAK83186.1, 239aa | 2e−72 | 136/227 (59.91%) | 160/227 (70.48%) | putative methyltransferase, Streptomyces |
| | | | | | | | viridochromogenes |
| | | | NP_419820.1, 453aa | 7e−08 | 52/188 (27.66%) | 81/188 (43.09%) | hypothetical protein, Caulobacter crescentus |
| 20 | HOXM | 481 | T30590, 491aa | 3e−55 | 147/470 (31.28%) | 236/470 (50.21%) | alkylhalidase homolog, Amycolatopsis |
| | | | | | | | orientalis |
| | | | CAA76550.1, 491aa | 4e−55 | 147/470 (31.28%) | 234/470 (49.79%) | halogenase, Amycolatopsis mediterranei |
| | | | AAK81830.1, 497aa | 9e−53 | 146/466 (31.33%) | 232/466 (49.79%) | non-heme halogenase, |
| | | | | | | | Streptomyces lavendulae |
| 21 | GTFA | 380 | AAD13553.1, 373aa | 2e−75 | 163/378 (43.12%) | 222/378 (58.73%) | glycosyl transferase homolog, |
| | | | | | | | Streptomyces cyanogenus |
| | | | AAF00209.1, 365aa | 6e−75 | 160/366 (43.72%) | 208/366 (56.83%) | glycosyl transferase, Streptomyces fradiae |
| | | | T46519, 382aa | 3e−67 | 162/383 (42.3%) | 209/383 (54.57%) | probable glycosyl transferase, |
| | | | | | | | Streptomyces violaceoruber |
| 22 | OXRU | 325 | AAK83190.1, 334aa | 6e−94 | 181/324 (55.86%) | 204/324 (62.96%) | putative pyruvate dehydrogenase, |
| | | | | | | | Streptomyces viridochromogenes |
| | | | NP_342958.1, 332aa | 1e−49 | 113/261 (43.3%) | 145/261 (55.56%) | pyruvate dehydrogenase, alpha subunit, |
| | | | | | | | Sulfolobus solfataricus |
| | | | NP_252839.1, 324aa | 3e−47 | 108/265 (40.75%) | 138/265 (52.08%) | probable dehydrogenase E1 |
| | | | | | | | component, Pseudomonas aeruginosa |
| 23 | OXRT | 320 | AAK83191.1, 320aa | 1e−138 | 247/320 (77.19%) | 275/320 (85.94%) | putative pyruvate dehydrogenase, |
| | | | | | | | Streptomyces viridochromogenes |
| | | | NP_342959.1, 324aa | 3e−59 | 133/325 (40.92%) | 195/325 (60%) | pyruvate dehydrogenase, beta subunit, |
| | | | | | | | Sulfolobus solfataricus |
| | | | NP_252840.1, 339aa | 6e−53 | 134/332 (40.36%) | 183/332 (55.12%) | acetoin catabolism protein, |
| | | | | | | | Pseudomonas aeruginosa |
| 24 | GTFE | 337 | AAK83192.1, 338aa | 1e−139 | 234/310 (75.48%) | 258/310 (83.23%) | putative glycosyltransferase, |
| | | | | | | | Streptomyces viridochromogenes |
| | | | NP_488533.1, 295aa | 1e−22 | 78/251 (31.08%) | 113/251 (45.02%) | probable glycosyltransferase, |
| | | | | | | | Nostoc sp. PCC 7120 |
| | | | NP_126869.1, 352aa | 2e−18 | 65/206 (31.55%) | 101/206 (49.03%) | rhamnosyl transferase related protein, |
| | | | | | | | Pyrococcus abyssi |
| 25 | UNKU | 350 | | | | | No homolog by blastp in GenBank nr |
| | | | | | | | protein database |
| 26 | OXRW | 252 | AAK83181.1, 223aa | 9e−14 | 70/236 (29.66%) | 103/236 (43.64%) | putative oxygenase, |
| | | | | | | | Streptomyces viridochromogenes |
| | | | AAF01812.1, 267aa | 8e−13 | 65/230 (28.26%) | 95/230 (41.3%) | SnoK, Streptomyces nogalater |
| | | | AAC71711.1, 286aa | 2e−11 | 54/228 (23.68%) | 98/228 (42.98%) | putative alpha-ketoglutarate-dependent |
| | | | | | | | hypophosphite dioxygenase, |
| | | | | | | | Pseudomonas stutzeri |
| 27 | DEPD | 309 | NP_254140.1, 323aa | 3e−46 | 123/320 (38.44%) | 171/320 (53.44%) | GDP-mannose 4,6-dehydratase, |
| | | | | | | | Pseudomonas aeruginosa |
| | | | NP_419826.1, 325aa | 6e−46 | 119/319 (37.3%) | 166/319 (52.04%) | GDP-mannose 4,6-dehydratase, |
| | | | | | | | Caulobacter crescentus |
| | | | AAC44117.1, 323aa | 5e−44 | 120/320 (37.5%) | 167/320 (52.19%) | GCA, Pseudomonas aeruginosa |
| 28 | NUTA | 355 | A26984, 355aa | 1e−127 | 216/353 (61.19%) | 276/353 (78.19%) | strD protein, Streptomyces griseus |
| | | | T30872, 355aa | 1e−125 | 214/354 (60.45%) | 279/354 (78.81%) | dNDP-glucose synthase, Streptomyces |
| | | | | | | | viridochromogenes |
| | | | AAD28517.1, 355aa | 1e−124 | 210/353 (59.49%) | 275/353 (77.9%) | BlmD, Streptomyces bluensis |
| 29 | DEPA | 329 | T30873, 355aa | 1e−139 | 239/319 (74.92%) | 265/319 (83.07%) | dNDP-glucose dehydratase, Streptomyces |
| | | | | | | | viridochromogenes |
| | | | AAG18457.1, 332aa | 1e−123 | 217/325 (66.77%) | 249/325 (76.62%) | AprE, Streptomyces tenebranus |
| | | | AAC68681.1, 337aa | 1e−115 | 207/329 (62.92%) | 247/329 (75.08%) | TDP-glucose-4,6-dehydratase, Streptomyces |
| | | | | | | | venezuelae |
| 30 | DEPJ | 342 | AAK83179.1, 342aa | 1e−157 | 268/342 (78.36%) | 293/342 (85.67%) | putative NDP-glucose 4-epimerase, |
| | | | | | | | Streptomyces viridochromogenes |
| | | | NP_228319.1, 309aa | 2e−40 | 113/305 (37.05%) | 155/305 (50.82%) | UDP-glucose 4-epimerase, putative, |
| | | | | | | | Thermotoga maritima |
| | | | NP_252757.1, 309aa | 4e−34 | 109/303 (35.97%) | 147/303 (48.51%) | probable epimerase, Pseudomonas aeruginosa |
| 31 | OXRX | 450 | AAK83181.1, 223aa | 5e−84 | 155/209 (74.16%) | 170/209 (81.34%) | putative oxygenase, Streptomyces |
| | | | | | | | viridochromogenes |
| | | | AAK83180.1, 199aa | 3e−64 | 124/198 (62.63%) | 143/198 (72.22%) | putative methyltransferase, Streptomyces |
| | | | | | | | viridochromogenes |
| | | | AAF01812.1, 267aa | 1e−10 | 57/212 (26.89%) | 90/212 (42.45%) | SnoK, Streptomyces nogalater |
| 32 | PKSO | 1267 | T30871, 1293aa | 0.0 | 758/1269 (59.73%) | 878/1269 (69.19%) | orsellinic acid synthase, Streptomyces |
| | | | | | | | viridochromogenes |
| | | | AAC49814.1, 1803aa | 0.0 | 452/1184 (38.18%) | 647/1184 (54.65%) | polyketide synthase, Aspergillus terreus |
| | | | AAK48943.1, 1778aa | 0.0 | 455/1194 (38.11%) | 639/1194 (53.52%) | 6-methylsalicylic acid synthase, |
| | | | | | | | Byssochlamys nivea |
| 33 | PHOD | 249 | NP_444200.1, 257aa | 9e−06 | 38/117 (32.48%) | 55/117 (47.01%) | p-nitrophenyl phosphatase, |
| | | | | | | | Halobacterium sp. |
| | | | AAG19324.1, 212aa | 9e−06 | 38/117 (32.48%) | 55/117 (47.01%) | p-nitrophenyl phosphatase; Pho2, |
| | | | | | | | Halobacterium sp. |
| 34 | DEPI | 307 | AAD45554.1, 312aa | 5e−53 | 125/288 (43.4%) | 160/288 (55.56%) | Spcl, Streptomyces netropsis |
| | | | NP_301272.1, 319aa | 8e−24 | 106/324 (32.72%) | 142/324 (43.83%) | putative sugar-nucleotide dehydratase, |
| | | | | | | | Mycobacterium leprae |
| | | | NP_228319.1, 309aa | 5e−23 | 86/303 (28.38%) | 136/303 (44.88%) | UDP-glucose 4-epimerase, putative, |
| | | | | | | | Thermotoga maritime |
| 35 | GTFG | 295 | AAK83170.1, 240aa | 3e−68 | 129/194 (66.49%) | 148/194 (76.29%) | putative glycosyltransferase, Streptomyces |
| | | | | | | | viridochromogenes |
| 36 | REBV | 82 | AAK83173.1, 192aa | 3e−23 | 51/76 (67.11%) | 63/76 (82.89%) | putative response regulator, Streptomyces |
| | | | | | | | viridochromogenes |
| | | | AAF71781.1, 210aa | 4e−22 | 47/70 (67.14%) | 63/70 (90%) | ORF4, Streptomyces noursei |
| | | | BAB69313.1, 232aa | 2e−19 | 44/70 (62.86%) | 57/70 (81.43%) | transcription regulatory protein, |
| | | | | | | | Streptomyces avermitilis |
| 37 | OXRA | 341 | AAF73453.1, 329aa | 3e−75 | 149/314 (47.45%) | 186/314 (59.24%) | putative 3-ketoreductase; AknQ, |
| | | | | | | | Streptomyces galilaeus |
| | | | AAA83425.2, 346aa | 5e−72 | 144/331 (43.5%) | 193/331 (58.31%) | RdmF, Streptomyces purpurascens |
| | | | AAD13550.1, 321aa | 1e−72 | 149/318 (46.86%) | 183/318 (57.55%) | oxidoreductase homolog, |
| | | | | | | | Streptomyces cyanogenus |
| 38 | DHYA | 470 | AAF59932.1, 474aa | 1e−122 | 246/480 (51.25%) | 303/480 (63.13%) | dTDP-4-keto-6-deoxyglucose |
| | | | | | | | 2,3-dehydratase, Streptomyces |
| | | | | | | | antibioticus |
| | | | CAB96551.1, 486aa | 1e−119 | 229/452 (50.66%) | 283/452 (62.61%) | D-olivose, D-oliose and D-mycarose 2,3- |
| | | | | | | | dehydratase, Streptomyces argillaceus |
| | | | AAG52988.1, 473aa | 1e−118 | 225/444 (50.68%) | 286/444 (64.41%) | Rif18, Amycolatopsis mediterranei |
| 39 | DEPE | 346 | CAC20923.1, 343aa | 1e−123 | 214/329 (65.05%) | 262/329 (79.64%) | PimJ protein, Streptomyces natalensis |
| | | | AAF71765.1, 344aa | 1e−120 | 209/330 (63.33%) | 258/330 (78.18%) | NysDIII, Streptomyces noursei |
| | | | AAK73500.1, 344aa | 1e−121 | 209/330 (63.33%) | 264/330 (80%) | AmphDIII, Streptomyces nodosus |
| 40 | MTLA | 254 | AAG32067.1, 250aa | 1e−37 | 93/249 (37.35%) | 116/249 (46.59%) | rRNA methyl transferase, Streptomyces |
| | | | | | | | viridochromogenes |
| 41 | DEPH | 160 | AAK83169.1, 322aa | 8e−43 | 90/159 (56.6%) | 105/159 (66.04%) | putative UDP-glucose 4-epimerase, |
| | | | | | | | Streptomyces viridochromogenes |
| | | | NP_228319.1, 309aa | 2e−30 | 70/162 (43.21%) | 98/162 (60.49%) | UDP-glucose 4-epimerase, putative, |
| | | | | | | | Thermotoga maritime |
| | | | NP_218151.1, 314aa | 1e−26 | 71/167 (42.51%) | 93/167 (55.69%) | rmlB2, Mycobacterium tuberculosis H37Rv |
| 42 | OXBN | 400 | S39965, 406aa | 7e−92 | 178/369 (48.24%) | 227/369 (61.52%) | hypothetical protein, Streptomyces griseus |
| | | | AAB63045.1, 342aa | 6e−44 | 100/216 (46.3%) | 118/216 (54.63%) | putative flavoprotein, Streptomyces peucetius |
| | | | NP_388333.1, 381aa | 1e−20 | 93/358 (25.98%) | 149/358 (41.62%) | similar to butyryl-CoA dehydrogenase, |
| | | | | | | | Bacillus subtilis |
| 43 | DATC | 375 | T30592, 369aa | 1e−150 | 263/373 (70.51%) | 297/373 (79.62%) | hypothetical protein, Amycolatopsis orientalis |
| | | | BAB72037.1, 369aa | 1e−146 | 262/373 (70.24%) | 289/373 (77.48%) | AclZ, Streptomyces galilaeus |
| | | | CAC48376.1, 369aa | 1e−146 | 255/373 (68.36%) | 292/373 (78.28%) | putative C-3 amino transferase, |
| | | | | | | | Amycolatopsis mediterranei |
| 44 | MTFV | 416 | CAC48364.1, 408aa | 1e−159 | 273/401 (68.08%) | 324/401 (80.8%) | putative C-3 methyl transferase, |
| | | | | | | | Amycolatopsis mediterranei |
| | | | AAC38444.1, 403aa | 1e−152 | 264/395 (66.84%) | 311/395 (78.73%) | daunorubicin/doxorubicin biosynthesis |
| | | | | | | | enzyme, Streptomyces peucetius |
| | | | AAB49295.1, 215aa | 7e−82 | 140/209 (66.99%) | 174/209 (83.25%) | hypothetical hydroxylase b, |
| | | | | | | | Amycolatopsis orientalis |
| 45 | EPIM | 207 | AAB63046.1, 208aa | 1e−60 | 114/194 (58.76%) | 136/194 (70.1%) | putative epimerase, Streptomyces peucetius |
| | | | S39966, 212aa | 2e−59 | 113/194 (58.25%) | 137/194 (70.62%) | hypothetical protein, Streptomyces griseus |
| | | | CAC48377.1, 205aa | 5e−59 | 110/198 (55.56%) | 140/198 (70.71%) | putative 3, 5 epimerase, |
| | | | | | | | Amycolatopsis mediterranei |
| 46 | DEPF | 369 | AAL14256.1, 325aa | 6e−64 | 138/312 (44.23%) | 170/312 (54.49%) | NDP-4-keto-6-deoxyhexose |
| | | | | | | | 4-ketoreductase, Streptomyces |
| | | | | | | | venezuelae |
| | | | AAG13913.1, 328aa | 5e−62 | 139/320 (43.44%) | 172/320 (53.75%) | TDP-4-keto-6-deoxyhexose 4-ketoreductase, |
| | | | | | | | Micromonospora megalomicea subsp. nigra |
| | | | BAA84595.1, 343aa | 9e−60 | 141/321 (43.93%) | 171/321 (53.27%) | dTDP-4-keto-6-deoxy-L-hexose 4-reductase, |
| | | | | | | | Streptomyces avermitilis |
| 47 | UNBB | 306 | | | | | No homolog by blastp in GenBank nr |
| | | | | | | | protein database |
| 48 | ENGA | 517 | P27035, 459aa | 3e−30 | 116/473 (24.52%) | 190/473 (40.17%) | endoglucanase, Streptomyces lividans |
| | | | CAC16970.1, 486aa | 4e−29 | 121/476 (25.42%) | 191/476 (40.13%) | putative secreted endoglucanase, |
| | | | | | | | Streptomyces coelicolor |
| | | | A55976, 596aa | 3e−25 | 61/113 (53.98%) | 70/113 (61.95%) | cellulose 1,4-beta-cellobiosidase, |
| | | | | | | | Thermomonospora fusca |
| 49 | REGL | 286 | T37231, 342aa | 9e−51 | 120/275 (43.64%) | 152/275 (55.27%) | probable transcription regulator, |
| | | | | | | | Streptomyces coelicolor |
| | | | CAB61919.1, 349aa | 2e−49 | 114/277 (41.16%) | 151/277 (54.51%) | putative lacl-family transcriptional regulator, |
| | | | | | | | Streptomyces coelicolor |
| | | | CAB56686.1, 347aa | 2e−40 | 105/279 (37.63%) | 138/279 (49.46%) | putative Lacl family transcriptional regulator, |
| | | | | | | | Streptomyces coelicolor |
| 50 | KINB | 423 | CAB65576.1, 406aa | 3e−37 | 109/274 (39.78%) | 134/274 (48.91%) | putative transcriptional regulatory protein, |
| | | | | | | | Streptomyces coelicolor |
| | | | NP_243624.1, 386aa | 2e−34 | 83/249 (33.33%) | 132/249 (53.01%) | transcriptional repressor, |
| | | | | | | | Bacillus halodurans |
| | | | T35673, 403aa | 4e−33 | 92/261 (35.25%) | 135/261 (51.72%) | probable transcription repressor, |
| | | | | | | | Streptomyces coelicolor |

NOTE:
For segmented ORF 41, only the N-terminal fragment was considered to generate this blast table.

TABLE II-B

| | | START (bp) . . . | LENGTH | | |
|---|---|---|---|---|---|
| EVER | ORF | END (bp) | (aa) | ORIENTATION | INTEGRITY |
| Contig 1 | 1 | 753 . . . 1 | 250 | NEGATIVE | COMPLETE |
| | 2 | 1533 . . . 2861 | 442 | POSITIVE | COMPLETE |
| | 3 | 2967 . . . 4055 | 362 | POSITIVE | COMPLETE |
| | 4 | 5529 . . . 4060 | 489 | NEGATIVE | COMPLETE |
| | 5 | 6174 . . . 6998 | 274 | POSITIVE | COMPLETE |
| | 6 | 6995 . . . 8284 | 429 | POSITIVE | COMPLETE |
| | 7 | 8281 . . . 9465 | 394 | POSITIVE | COMPLETE |
| | 8 | 9472 . . . 10491 | 339 | POSITIVE | COMPLETE |
| | 9 | 10607 . . . 11020 | 137 | POSITIVE | COMPLETE |
| | 10 | 12020 . . . 11076 | 314 | NEGATIVE | COMPLETE |
| | 11 | 13089 . . . 12061 | 342 | NEGATIVE | COMPLETE |
| | 12 | 13912 . . . 13082 | 276 | NEGATIVE | COMPLETE |
| | 13 | 14812 . . . 14015 | 265 | NEGATIVE | COMPLETE |
| | 14 | 15177 . . . 16211 | 344 | POSITIVE | COMPLETE |
| | 15 | 17099 . . . 16377 | 240 | NEGATIVE | COMPLETE |
| | 16 | 18415 . . . 17099 | 438 | NEGATIVE | COMPLETE |
| | 17 | 19900 . . . 18683 | 405 | NEGATIVE | COMPLETE |
| | 18 | 20858 . . . 19917 | 313 | NEGATIVE | COMPLETE |
| | 19 | 21589 . . . 20858 | 243 | NEGATIVE | COMPLETE |
| | 20 | 23031 . . . 21586 | 481 | NEGATIVE | COMPLETE |
| | 21 | 23345 . . . 24487 | 380 | POSITIVE | COMPLETE |
| | 22 | 24565 . . . 25542 | 325 | POSITIVE | COMPLETE |
| | 23 | 25547 . . . 26509 | 320 | POSITIVE | COMPLETE |
| | 24 | 26557 . . . 27570 | 337 | POSITIVE | COMPLETE |
| | 25 | 27567 . . . 28619 | 350 | POSITIVE | COMPLETE |
| | 26 | 29397 . . . 28639 | 252 | NEGATIVE | COMPLETE |
| | 27 | 29752 . . . 30681 | 309 | POSITIVE | COMPLETE |
| | 28 | 30879 . . . 31946 | 355 | POSITIVE | COMPLETE |
| | 29 | 31946 . . . 32935 | 329 | POSITIVE | COMPLETE |
| | 30 | 32990 . . . 34018 | 342 | POSITIVE | COMPLETE |
| | 31 | 34073 . . . 35425 | 450 | POSITIVE | COMPLETE |
| | 32 | 39383 . . . 35580 | 1267 | NEGATIVE | COMPLETE |
| | 33 | 39863 . . . 40612 | 249 | POSITIVE | COMPLETE |
| | 34 | 40609 . . . 41532 | 307 | POSITIVE | COMPLETE |
| | 35 | 42398 . . . 41511 | 295 | NEGATIVE | COMPLETE |
| | 36 | 42708 . . . 42460 | 82 | NEGATIVE | COMPLETE |
| | 37 | 44557 . . . 43532 | 341 | NEGATIVE | COMPLETE |
| | 38 | 45966 . . . 44554 | 470 | NEGATIVE | COMPLETE |
| | 39 | 47003 . . . 45963 | 346 | NEGATIVE | COMPLETE |
| | 40 | 47971 . . . 47207 | 254 | NEGATIVE | COMPLETE |
| | 41 | 48221 . . . 48070 | 49 | NEGATIVE | C-TERMINUS ONLY |
| Contig 2 | 41 | 480 . . . 1 | 160 | NEGATIVE | N-TERMINUS ONLY |
| Contig 3 | 42 | 1 . . . 1203 | 400 | POSITIVE | C-TERMINUS ONLY |
| | 43 | 1200 . . . 2327 | 375 | POSITIVE | COMPLETE |
| | 44 | 2357 . . . 3607 | 416 | POSITIVE | COMPLETE |
| | 45 | 3616 . . . 4239 | 207 | POSITIVE | COMPLETE |
| | 46 | 5169 . . . 4060 | 369 | NEGATIVE | COMPLETE |
| | 47 | 6086 . . . 5166 | 306 | NEGATIVE | COMPLETE |
| | 48 | 7811 . . . 6261 | 517 | NEGATIVE | COMPLETE |
| | 49 | 8746 . . . 7889 | 286 | NEGATIVE | COMPLETE |
| | 50 | 10035 . . . 8764 | 423 | NEGATIVE | COMPLETE |

### EXAMPLE 2

### Identification of a Biosynthetic Locus for an Avilamycin-Type Compound from Streptomyces mobaraensis

The microorganism Streptomyces mobarensis strain NRRL B-3729 was obtained from the Agriculture Research Service Culture Collection of the United States Department of Agriculture. Streptomyces mobarensis was not previously reported to produce an avilamycin-type compound or orthosomycins in general. A biosynthetic locus for an avilamycin-type compound in Streptomyces mobarensis (AVIA) was identified using the method described in Canadian patent application CA 2,352,451. The sequences obtained from cosmids containing overlapping genomic inserts spanning the biosynthetic locus for everninomicin were identified. Within the sequences of the cosmid inserts, numerous ORFs encoding polypeptides having homology to known proteins were identified. Homology was determined using the program BLASTP version 2.2.2 with the default parameters. A contiguous nucleotide sequence spanning AVIA and deduced amino acid sequences of AVIA are provided as follows: The amino acid sequence of ORF 1 (SEQ ID NO 123) is deduced from the nucleic acid sequence of SEQ ID NO 124 drawn from contig 1 (SEQ ID NO 277). The amino acid sequence of ORF 2 (SEQ ID NO 203) is deduced from the nucleic acid sequence of SEQ ID NO 204 drawn from contig 1 (SEQ ID NO 277). The amino acid sequence of ORF 3 (SEQ ID NO 127) is deduced from the nucleic acid sequence of SEQ ID NO 128 drawn from contig 1 (SEQ ID NO 277). The amino acid sequence of ORF 4 (SEQ ID NO 19) is deduced from the nucleic acid sequence of SEQ ID NO 20 drawn from contig 1 (SEQ ID NO 277). The amino acid sequence of ORF 5 (SEQ ID NO 57) is deduced from the nucleic acid sequence of SEQ ID NO 58 drawn from contig 1 (SEQ ID NO 277). The amino acid sequence of ORF 6 (SEQ ID NO 253) is deduced from the nucleic acid sequence of SEQ ID NO 254 drawn from contig 1 (SEQ ID NO 277). The amino acid sequence of ORF 7 (SEQ ID NO 251) is deduced from the nucleic acid sequence of SEQ ID NO 252 drawn from contig 1 (SEQ ID NO 277). The amino acid sequence of ORF 8 (SEQ ID NO 187) is deduced from the nucleic acid sequence of SEQ ID NO 188 drawn from contig 1 (SEQ ID NO 277). The amino acid sequence of ORF 9 (SEQ ID NO 199) is deduced from the nucleic acid sequence of SEQ ID NO 200 drawn from contig 1 (SEQ ID NO 277). The amino acid sequence of ORF 10 (SEQ ID NO 255) is deduced from the nucleic acid sequence of SEQ ID NO 256 drawn from contig 1 (SEQ ID NO 277). The amino acid sequence of ORF 11 (SEQ ID NO 117) is deduced from the nucleic acid sequence of SEQ ID NO 118 drawn from contig 1 (SEQ ID NO 277). The amino acid sequence of ORF 12 (SEQ ID NO 87) is deduced from the nucleic acid sequence of SEQ ID NO 88 drawn from contig 1 (SEQ ID NO 277). The amino acid sequence of ORF 13 (SEQ ID NO 81) is deduced from the nucleic acid sequence of SEQ ID NO 82 drawn from contig 1 (SEQ ID NO 277). The amino acid sequence of ORF 14 (SEQ ID NO 181) is deduced from the nucleic acid sequence of SEQ ID NO 182 drawn from contig 1 (SEQ ID NO 277). The amino acid sequence of ORF 15 (SEQ ID NO 133) is deduced from the nucleic acid sequence of SEQ ID NO 134 drawn from contig 1 (SEQ ID NO 277). The amino acid sequence of ORF 16 (SEQ ID NO 1) is deduced from the nucleic acid sequence of SEQ ID NO 2 drawn from contig 1 (SEQ ID NO 277). The amino acid sequence of ORF 17 (SEQ ID NO 33) is deduced from the nucleic acid sequence of SEQ ID NO 34 drawn from contig 1 (SEQ ID NO 277). The amino acid sequence of ORF 18 (SEQ ID NO 165) is deduced from the nucleic acid sequence of SEQ ID NO 166 drawn from contig 1 (SEQ ID NO 277). The amino acid sequence of ORF 19 (SEQ ID NO 167) is deduced from the nucleic acid sequence of SEQ ID NO 168 drawn from contig 1 (SEQ ID NO 277). The amino acid sequence of ORF 20 (SEQ ID NO 45) is deduced from the nucleic acid sequence of SEQ ID NO 46 drawn from contig 1 (SEQ ID NO 277). The amino acid sequence of ORF 21 (SEQ ID NO 247) is deduced from the nucleic acid sequence of SEQ ID NO 248 drawn from contig 1 (SEQ ID NO 277). The amino acid sequence of ORF 22 (SEQ ID NO 99) is deduced from the nucleic acid sequence of SEQ ID NO 100 drawn from contig 1 (SEQ ID NO 277). The amino acid sequence of ORF 23 (SEQ ID NO 105) is deduced from the nucleic acid sequence of SEQ ID NO 106 drawn from contig 1 (SEQ ID NO 277). The amino acid sequence of ORF 24 (SEQ ID NO 153) is deduced from the nucleic acid sequence of SEQ ID NO 154 drawn from contig 1 (SEQ ID NO 277). The amino acid sequence of ORF 25 (SEQ ID NO 111) is deduced from the nucleic acid sequence of SEQ ID NO 112 drawn from contig 1 (SEQ ID NO 277). The amino acid sequence of ORF 26 (SEQ ID NO 193) is deduced from the nucleic acid sequence of SEQ ID NO 194 drawn from contig 1 (SEQ ID NO 277). The amino acid sequence of ORF 27 (SEQ ID NO 245) is deduced from the nucleic acid sequence of SEQ ID NO 246 drawn from contig 1 (SEQ ID NO 277). The amino acid sequence of ORF 28 (SEQ ID NO 249) is deduced from the nucleic acid sequence of SEQ ID NO 250 drawn from contig 1 (SEQ ID NO 277). The amino acid sequence of ORF 29 (SEQ ID NO 149) is deduced from the nucleic acid sequence of SEQ ID NO 150 drawn from contig 1 (SEQ ID NO 277). The amino acid sequence of ORF 30 (SEQ ID NO 145) is deduced from the nucleic acid sequence of SEQ ID NO 146 drawn from contig 1 (SEQ ID NO 277). The amino acid sequence of ORF 31 (SEQ ID NO 51) is deduced from the nucleic acid sequence of SEQ ID NO 52 drawn from contig 1 (SEQ ID NO 277). The amino acid sequence of ORF 32 (SEQ ID NO 63) is deduced from the nucleic acid sequence of SEQ ID NO 64 drawn from contig 1 (SEQ ID NO 277). The amino acid sequence of ORF 33 (SEQ ID NO 159) is deduced from the nucleic acid sequence of SEQ ID NO 160 drawn from contig 1 (SEQ ID NO 277). The amino acid sequence of ORF 34 (SEQ ID NO 175) is deduced from the nucleic acid sequence of SEQ ID NO 176 drawn from contig 1 (SEQ ID NO 277). The amino acid sequence of ORF 35 (SEQ ID NO 27) is deduced from the nucleic acid sequence of SEQ ID NO 28 drawn from contig 1 (SEQ ID NO 277). The amino acid sequence of ORF 36 (SEQ ID NO 75) is deduced from the nucleic acid sequence of SEQ ID NO 76 drawn from contig 1 (SEQ ID NO 277). The amino acid sequence of ORF 37 (SEQ ID NO 69) is deduced from the nucleic acid sequence of SEQ ID NO 70 drawn from contig 1 (SEQ ID NO 277). The amino acid sequence of ORF 38 (SEQ ID NO 93) is deduced from the nucleic acid sequence of SEQ ID NO 94 drawn from contig 1 (SEQ ID NO 277). The amino acid sequence of ORF 39 (SEQ ID NO 7) is deduced from the nucleic acid sequence of SEQ ID NO 8 drawn from contig 1 (SEQ ID NO 277). The amino acid sequence of ORF 40 (SEQ ID NO 39) is deduced from the nucleic acid sequence of SEQ ID NO 40 drawn from contig 1 (SEQ ID NO 277). The amino acid sequence of ORF 41 (SEQ ID NO 139) is deduced from the nucleic acid sequence of SEQ ID NO 140 drawn from contig 1 (SEQ ID NO 277). The amino acid sequence of ORF 42 (SEQ ID NO 13) is deduced from the nucleic acid sequence of SEQ ID NO 14 drawn from contig 1 (SEQ ID NO 277). The ORFs in AVIA have been assigned a putative function and protein family designation based on homology to known proteins as indicated in Table III-A. The position, length and orientation of each AVIA ORF within SEQ ID NO: 277 is provided in Table III-B

TABLE III-A
AVIA blast table

| ORF | Family | #aa | GenBank homology | probability | | | |
|---|---|---|---|---|---|---|---|
| 1 | MTIA | 285 | AAG32066.1, 287aa | 1e−133 | 234/273 (85.71%) | 246/273 (90.11%) | rRNA methyltransferase, |
| | | | | | | | Streptomyces viridochromogenes |
| | | | NP_540715.1, 297aa | 4e−24 | 82/257 (31.91%) | 125/257 (48.64%) | 23S ribosomal RNA methyltransferase, |
| | | | | | | | Brucella melitensis |
| | | | NP_336137.1, 260aa | 1e−22 | 80/268 (29.85%) | 125/268 (46.64%) | rRNA methylase, putative, |
| | | | | | | | Mycobacterium tuberculosis |
| 2 | UNKU | 343 | | | | | No homolog by blastp in GenBank nr |
| | | | | | | | protein database |
| 3 | MTLA | 250 | AAG32067.1, 250aa | 2e−88 | 170/236 (72.03%) | 181/236 (76.69%) | rRNA methyl transferase, |
| | | | | | | | Streptomyces viridochromogenes |
| 4 | DEPH | 320 | AAK83169.1, 322aa | 1e−149 | 265/320 (82.81%) | 277/320 (86.56%) | putative UDP-glucose 4-epimerase, |
| | | | | | | | Streptomyces viridochromogenes |
| | | | NP_228319.1, 309aa | 3e−62 | 133/303 (43.89%) | 180/303 (59.41%) | UDP-glucose 4-epimerase, putative, |
| | | | | | | | Thermotoga maritima pir |
| | | | NP_247180.1, 305aa | 1e−60 | 132/305 (43.28%) | 184/305 (60.33%) | UDP-glucose 4-epimerase (galE), |
| | | | | | | | Methanococcus jannaschii |
| 5 | GTFG | 294 | AAK83170.1, 240aa | 1e−94 | 170/193 (88.08%) | 181/193 (93.78%) | putative glycosyltransferase, |
| | | | | | | | Streptomyces viridochromogenes |
| | | | NP_487216.1, 313aa | 1e−05 | 58/221 (26.24%) | 84/221 (38.01%) | probable glycosyl transferase, |
| | | | | | | | Nostoc sp. |
| | | | AAB88894.1, 327aa | 5e−04 | 54/218 (24.77%) | 84/218 (38.53%) | putative glycosyl transferase, |
| | | | | | | | Rhizobium leguminosarum |
| 6 | UNAI | 356 | AAK83171.1, 355aa | 1e−173 | 294/355 (82.82%) | 316/355 (89.01%) | AviX9, Streptomyces |
| | | | | | | | viridochromogenes |
| | | | CAB61298.1, 391aa | 3e−25 | 86/265 (32.45%) | 126/265 (47.55%) | hypothetical protein, |
| | | | | | | | Streptomyces coelicolor |
| 7 | REBU | 99 | AAK83172.1, 206aa | 8e−26 | 60/65 (92.31%) | 61/65 (93.85%) | putative two-component regulator, |
| | | | | | | | Streptomyces viridochromogenes |
| | | | NP_372408.1, 209aa | 5e−10 | 29/61 (47.54%) | 45/61 (73.77%) | two-component response regulator, |
| | | | | | | | Staphylococcus aureus |
| | | | T36862, 210aa | 4e−09 | 31/62 (50%) | 42/62 (67.74%) | probable two-component response |
| | | | | | | | regulator, Streptomyces coelicolor |
| 8 | REBV | 158 | AAK83173.1, 192aa | 4e−38 | 77/87 (88.51%) | 83/87 (95.4%) | putative response regulator, |
| | | | | | | | Streptomyces viridochromogenes |
| | | | AAF71781.1, 210aa | 2e−18 | 44/82 (53.66%) | 62/82 (75.61%) | ORF4, Streptomyces noursei |
| | | | BAB69313.1, 232aa | 3e−16 | 40/73 (54.79%) | 54/73 (73.97%) | transcription regulatory protein, |
| | | | | | | | Streptomyces avermitilis |
| 9 | UEVB | 136 | AAK83174.1, 136aa | 1e−75 | 126/135 (93.33%) | 134/135 (99.26%) | AviX10, Streptomyces viridochromogenes |
| 10 | UNBR | 249 | AAK83175.1, 231aa | 1e−114 | 195/216 (90.28%) | 205/216 (94.91%) | AviX11, Streptomyces viridochromogenes |
| 11 | MTFV | 407 | AAK83176.1, 407aa | 0.0 | 361/407 (88.7%) | 379/407 (93.12%) | methyltransferase, Streptomyces |
| | | | | | | | viridochromogenes |
| | | | AAD41823.1, 418aa | 1e−142 | 261/409 (63.81%) | 297/409 (72.62%) | NDP-hexose 3-C-methyltransferase, |
| | | | | | | | Streptomyces fradiae |
| 12 | MEMK | 456 | AAK83177.1, 476aa | 1e−141 | 268/397 (67.51%) | 286/397 (72.04%) | putative transporter, Streptomyces |
| | | | | | | | viridochromogenes |
| | | | NP_440311.1, 410aa | 1e−49 | 125/380 (32.89%) | 186/380 (48.95%) | Na/H antiporter, Synechocystis sp. |
| | | | AAL79755.1, 780aa | 8e−45 | 125/379 (32.98%) | 175/379 (46.17%) | putative ion antiporter, Oryza sativa |
| 13 | KASA | 344 | AAK83178.1, 343aa | 1e−163 | 292/346 (84.39%) | 305/346 (88.15%) | AviN, Streptomyces viridochromogenes |
| | | | AAG29787.1, 355aa | 4e−75 | 151/337 (44.81%) | 202/337 (59.94%) | 3-ketoacyl-ACP-synthase, Streptomyces |
| | | | | | | | rishiriensis |
| | | | CAB71914.1, 346aa | 3e−67 | 135/345 (39.13%) | 202/345 (58.55%) | hypothetical protein, Streptomyces coelicolor |
| 14 | PKSO | 1284 | T30871, 1293aa | 0.0 | 1002/1293 (77.49%) | 1049/1293 (81.13%) | orsellinic acid synthase, Streptomyces |
| | | | | | | | viridochromogenes |
| | | | BAA20102.1, 1800aa | 0.0 | 439/1214 (36.16%) | 601/1214 (49.51%) | 6-methylsalicylic acid synthase, Aspergillus |
| | | | | | | | terreus |
| | | | AAK48943.1, 1778aa | 0.0 | 420/1170 (35.9%) | 589/1170 (50.34%) | 6-methylsalicylic acid synthase, Byssochlamys |
| | | | | | | | nivea |
| 15 | NUTA | 355 | T30872, 355aa | 1e−178 | 314/354 (88.7%) | 338/354 (95.48%) | dNDP-glucose synthase, |
| | | | | | | | Streptomyces viridochromogenes |
| | | | A26984, 355aa | 1e−122 | 223/353 (63.17%) | 269/353 (76.2%) | strD protein, Streptomyces griseus |
| | | | T48866, 355aa | 1e−120 | 216/353 (61.19%) | 265/353 (75.07%) | glucose-1-phosphate thymidylyltransferase, |
| | | | | | | | Streptomyces argillaceus |
| 16 | DEPA | 346 | T30873, 355aa | 1e−176 | 305/337 (90.5%) | 313/337 (92.88%) | dNDP-glucose dehydratase, Streptomyces |
| | | | | | | | viridochromogenes |
| | | | AAG18457.1, 332aa | 1e−117 | 212/329 (64.44%) | 242/329 (73.56%) | AprE, Streptomyces tenebrarius |
| | | | AAD31797.1, 330aa | 1e−115 | 206/328 (62.8%) | 240/328 (73.17%) | TDP-glucose-4,6-dehydratase, Streptomyces |
| | | | | | | | spectabilis |
| 17 | DEPJ | 342 | AAK83179.1, 342aa | 0.0 | 321/342 (93.86%) | 325/342 (95.03%) | putative NDP-glucose 4-epimerase, |
| | | | | | | | Streptomyces viridochromogenes |
| | | | NP_252757.1, 309aa | 1e−38 | 113/302 (37.42%) | 156/302 (51.66%) | probable epimerase, |
| | | | | | | | Pseudomonas aeruginosa |
| | | | NP_228319.1, 309aa | 9e−43 | 113/305 (37.05%) | 166/305 (54.43%) | UDP-glucose 4-epimerase, |
| | | | | | | | putative, Thermotoga maritima |
| 18 | UNAJ | 222 | AAK83180.1, 199aa | 2e−88 | 162/198 (81.82%) | 174/198 (87.88%) | putative methyltransferase, |
| | | | | | | | Streptomyces viridochromogenes |
| 19 | OXRW | 223 | AAK83181.1, 223aa | 1e−106 | 190/223 (85.2%) | 192/223 (86.1%) | putative oxygenase, |
| | | | | | | | Streptomyces viridochromogenes |
| 20 | GTFA | 430 | AAK83182.1, 430aa | 0.0 | 390/430 (90.7%) | 410/430 (95.35%) | putative glycosyltransferase, |
| | | | | | | | Streptomyces viridochromogenes |
| | | | T46681, 411aa | 9e−67 | 159/415 (38.31%) | 218/415 (52.53%) | probable glycosyltransferase, |
| | | | | | | | Streptomyces nogalater |
| | | | AAC64928.1, 393aa | 3e−47 | 148/430 (34.42%) | 198/430 (46.05%) | glycosyltransferase, |
| | | | | | | | Streptomyces argillaceus |
| 21 | DEPN | 337 | AAK83183.1, 337aa | 1e−174 | 301/337 (89.32%) | 320/337 (94.96%) | putative NDP-glucose 4,6-dehydratase, |
| | | | | | | | Streptomyces viridochromogenes |
| | | | T35486, 330aa | 4e−95 | 175/311 (56.27%) | 216/311 (69.45%) | probable nucleotide-sugar dehydratase, |
| | | | | | | | Streptomyces coelicolor |
| | | | S58282, 445aa | 2e−82 | 160/307 (52.12%) | 201/307 (65.47%) | dTDP-glucose 4-6-dehydratase |
| | | | | | | | homolog, Arabidopsis thaliana |
| 22 | MTFD | 242 | AAK83184.1, 240aa | 1e−122 | 216/240 (90%) | 222/240 (92.5%) | putative methyltransferase, |
| | | | | | | | Streptomyces viridochromogenes |
| 23 | MTFE | 238 | AAK83186.1, 239aa | 1e−119 | 210/231 (90.91%) | 216/231 (93.51%) | putative methyltransferase, |
| | | | | | | | Streptomyces viridochromogenes |
| | | | BAB69329.1, 266aa | 3e−07 | 53/155 (34.19%) | 63/155 (40.65%) | putative methyltransferase, |
| | | | | | | | Streptomyces avermitilis |
| 24 | OXRV | 314 | AAK83187.1, 314aa | 1e−1 63 | 270/314 (85.99%) | 285/314 (90.76%) | putative oxygenase, |
| | | | | | | | Streptomyces viridochromogenes |
| 25 | MTFF | 278 | AAK83188.1, 277aa | 1e−122 | 211/276 (76.45%) | 223/276 (80.8%) | AviG3, Streptomyces viridochromogenes |
| | | | NP_294079.1, 173aa | 2e−05 | 24/43 (55.81%) | 30/43 (69.77%) | methyltransferase, putative, |
| | | | | | | | Deinococcus radiodurans |
| 26 | UEVA | 396 | AAK83189.1, 396aa | 0.0 | 379/395 (95.95%) | 386/395 (97.72%) | AviX12, Streptomyces viridochromogenes |
| 27 | ABCD | 323 | AAG32068.1, 323aa | 1e−147 | 268/323 (82.97%) | 278/323 (86.07%) | putative ATP binding protein, |
| | | | | | | | Streptomyces viridochromogenes |
| | | | CAC08315.1, 336aa | 7e−95 | 184/309 (59.55%) | 216/309 (69.9%) | putative ABC transport system ATP-binding |
| | | | | | | | protein, Streptomyces coelicolor |
| | | | T36344, 315aa | 4e−85 | 172/311 (55.31%) | 207/311 (66.56%) | probable ABC-type transport system |
| | | | | | | | ATP-binding protein, Streptomyces coelicolor |
| 28 | MEMD | 264 | AAG32069.1, 265aa | 1e−122 | 216/264 (81.82%) | 219/264 (82.95%) | putative transmembrane protein, |
| | | | | | | | Streptomyces viridochromogenes |
| | | | CAC08316.1, 280aa | 1e−52 | 108/263 (41.06%) | 153/263 (58.17%) | putative ABC transport system integral |
| | | | | | | | membrane protein, |
| | | | | | | | Streptomyces coelicolor |
| | | | T36343, 260aa | 6e−46 | 95/261 (36.4%) | 141/261 (54.02%) | probable ABC-type transport system |
| | | | | | | | transmembrane protein, |
| | | | | | | | Streptomyces coelicolor |
| 29 | OXRU | 325 | AAK83190.1, 334aa | 1e−140 | 248/325 (76.31%) | 260/325 (80%) | putative pyruvate dehydrogenase, Streptomyces |
| | | | | | | | viridochromogenes |
| | | | NP_342958.1, 332aa | 3e−56 | 126/328 (38.41%) | 175/328 (53.35%) | Pyruvate dehydrogenase-alpha subunit, |
| | | | | | | | Sulfolobus solfataricus |
| | | | I40790, 326aa | 2e−53 | 121/320 (37.81%) | 159/320 (49.69%) | acetoin dehydrogenase-alpha chain, |
| | | | | | | | Clostridium magnum |
| 30 | OXRT | 320 | AAK83191.1, 320aa | 1e−163 | 293/320 (91.56%) | 296/320 (92.5%) | putative pyruvate dehydrogenase, |
| | | | | | | | Streptomyces viridochromogenes |
| | | | NP_342959.1, 324aa | 4e−58 | 134/325 (41.23%) | 190/325 (58.46%) | Pyruvate dehydrogenase-beta subunit, |
| | | | | | | | Sulfolobus solfataricus |
| | | | I40791, 333aa | 4e−50 | 124/337 (36.8%) | 184/337 (54.6%) | acetoin dehydrogenase-beta chain, |
| | | | | | | | Clostridium magnum |
| 31 | GTFE | 335 | AAK83192.1, 338aa | 1e−169 | 285/335 (85.07%) | 299/335 (89.25%) | putative glycosyltransferase, |
| | | | | | | | Streptomyces viridochromogenes |
| | | | NP_349669.1, 299aa | 7e−19 | 67/230 (29.13%) | 103/230 (44.78%) | Predicted glycosyltransferase, |
| | | | | | | | Clostridium acetobutylicum |
| | | | NP_348928.1, 303aa | 7e−18 | 64/266 (24.06%) | 116/266 (43.61%) | Rhamnosyl transferase, |
| | | | | | | | Clostridium acetobutylicum |
| 32 | GTFH | 342 | AAK83193.1, 342aa | 1e−156 | 272/342 (79.53%) | 281/342 (82.16%) | putative glycosyltransferase, |
| | | | | | | | Streptomyces viridochromogenes |
| | | | NP_489347.1, 367aa | 4e−12 | 84/325 (25.85%) | 121/325 (37.23%) | probable glycosyl transferase, Nostoc sp. |
| | | | NP_102448.1, 353aa | 2e−11 | 93/352 (26.42%) | 130/352 (36.93%) | probable sugar transferase, Mesorhizobium loti |
| 33 | OXRW | 250 | AAK83181.1, 223aa | 1e−09 | 63/234 (26.92%) | 93/234 (39.74%) | putative oxygenase, Streptomyces |
| | | | | | | | viridochromogenes |
| 34 | PHOD | 241 | NP_417844.1, 252aa | 8e−06 | 43/134 (32.09%) | 68/134 (50.75%) | phosphoglycolate phosphatase, |
| | | | | | | | Escherichia coli K12 |
| 35 | DEPI | 314 | AAD45554.1, 312aa | 7e−46 | 115/284 (40.49%) | 143/284 (50.35%) | Spcl, Streptomyces netropsis |
| | | | AAK83169.1, 322aa | 4e−18 | 94/308 (30.52%) | 129/308 (41.88%) | putative UDP-glucose 4-epimerase, |
| | | | | | | | Streptomyces viridochromogenes |
| | | | NP_338283.1, 322aa | 4e−18 | 94/307 (30.62%) | 122/307 (39.74%) | NAD-dependent epimerase/dehydratase, |
| | | | | | | | Mycobacterium tuberculosis |
| 36 | HOXM | 485 | T44858, 491aa | 3e−65 | 154/428 (35.98%) | 233/428 (54.44%) | probable hydroxylase, Amycolatopsis orientalis |
| | | | T30590, 491aa | 1e−64 | 157/429 (36.6%) | 233/429 (54.31%) | alkylhalidase homolog, Amycolatopsis orientalis |
| | | | AAK81830.1, 497aa | 4e−63 | 150/416 (36.06%) | 225/416 (54.09%) | non-heme halogenase, Streptomyces lavendulae |
| 37 | HOXG | 283 | | | | | No homolog by blastp in GenBank nr |
| | | | | | | | protein database |
| 38 | MTFA | 348 | BAA32132.1, 305aa | 1e−57 | 127/306 (41.5%) | 150/306 (49.02%) | Orf4, Streptomyces griseus |
| | | | P42712, 376aa | 6e−57 | 129/333 (38.74%) | 170/333 (51.05%) | O-demethylpuromycin-O-methyltransferase, |
| | | | | | | | Streptomyces alboniger |
| | | | CAB76315.1, 342aa | 2e−24 | 79/286 (27.62%) | 121/286 (42.31%) | putative O-methyltransferase, |
| | | | | | | | Streptomyces coelicolor |
| 39 | DEPE | 348 | CAC20923.1, 343aa | 1e−124 | 213/326 (65.34%) | 258/326 (79.14%) | PimJ protein, Streptomyces natalensis |
| | | | AAK73500.1, 344aa | 1e−122 | 212/326 (65.03%) | 261/326 (80.06%) | AmphDIII, Streptomyces nodosus |
| | | | AAF71765.1, 344aa | 1e−120 | 211/341 (61.88%) | 263/341 (77.13%) | NysDIII, Streptomyces noursei |
| 40 | DHYA | 466 | CAB96551.1, 486aa | 1e−130 | 247/479 (51.57%) | 306/479 (63.88%) | D-olivose, D-oliose and D-mycarose2,3- |
| | | | | | | | dehydratase, Streptomyces argillaceus |
| | | | AAF59932.1, 474aa | 1e−127 | 235/444 (52.93%) | 290/444 (65.32%) | dTDP-4-keto-6-deoxyglucose 2,3-dehydratase, |
| | | | | | | | Streptomyces antibioticus |
| | | | T46668, 488aa | 1e−121 | 238/476 (50%) | 295/476 (61.97%) | probable 2,3-dehydratase, Streptomyces |
| | | | | | | | nogalater emb |
| 41 | OXRA | 336 | CAB96550.1, 328aa | 5e−82 | 151/280 (53.93%) | 191/280 (68.21%) | D-oliose 4-ketoreductase, Streptomyces |
| | | | | | | | argillaceus |
| | | | AAF73453.1, 329aa | 2e−80 | 165/323 (51.08%) | 196/323 (60.68%) | putative 3-ketoreductase, Streptomyces galilaeus |
| | | | AAD13550.1, 321aa | 4e−78 | 152/285 (53.33%) | 186/285 (65.26%) | oxidoreductase homolog, Streptomyces |
| | | | | | | | cyanogenus |
| 42 | DEPG | 324 | AAD13561.1, 326aa | 4e−31 | 105/316 (33.23%) | 135/316 (42.72%) | NDP-hexose 4-keto reductase homolog, |
| | | | | | | | Streptomyces cyanogenus |
| | | | T46527, 304aa | 7e−31 | 104/312 (33.33%) | 139/312 (44.55%) | probable dTDP4-keto-6-deoxyhexose |
| | | | | | | | reductase, Streptomyces violaceoruber |
| | | | AAB63047.1, 307aa | 5e−29 | 95/310 (30.65%) | 132/310 (42.58%) | thymidine diphospho-4-keto-2,3,6- |
| | | | | | | | trideoxyhexulose reductase, |
| | | | | | | | Streptomyces peucetius |

TABLE III-B

| | | | LENGTH | | |
|---|---|---|---|---|---|
| AVIA | ORF | START (bp) . . . END (bp) | (aa) | ORIENTATION | INTEGRITY |
| Contig 1 | 1 | 1 . . . 858 | 285 | POSITIVE | COMPLETE |
| | 2 | 1847 . . . 816 | 343 | NEGATIVE | COMPLETE |
| | 3 | 2721 . . . 1969 | 250 | NEGATIVE | COMPLETE |
| | 4 | 2972 . . . 3934 | 320 | POSITIVE | COMPLETE |
| | 5 | 4870 . . . 3986 | 294 | NEGATIVE | COMPLETE |
| | 6 | 5044 . . . 6114 | 356 | POSITIVE | COMPLETE |
| | 7 | 6410 . . . 6111 | 99 | NEGATIVE | COMPLETE |
| | 8 | 7412 . . . 6936 | 158 | NEGATIVE | COMPLETE |
| | 9 | 7639 . . . 8049 | 136 | POSITIVE | COMPLETE |
| | 10 | 7992 . . . 8741 | 249 | POSITIVE | COMPLETE |
| | 11 | 8738 . . . 9961 | 407 | POSITIVE | COMPLETE |
| | 12 | 10020 . . . 11390 | 456 | POSITIVE | COMPLETE |
| | 13 | 11546 . . . 12580 | 344 | POSITIVE | COMPLETE |
| | 14 | 12577 . . . 16431 | 1284 | POSITIVE | COMPLETE |
| | 15 | 16623 . . . 17690 | 355 | POSITIVE | COMPLETE |
| | 16 | 17723 . . . 18763 | 346 | POSITIVE | COMPLETE |
| | 17 | 18842 . . . 19870 | 342 | POSITIVE | COMPLETE |
| | 18 | 19960 . . . 20676 | 222 | POSITIVE | COMPLETE |
| | 19 | 20723 . . . 21394 | 223 | POSITIVE | COMPLETE |
| | 20 | 21442 . . . 22734 | 430 | POSITIVE | COMPLETE |
| | 21 | 22731 . . . 23744 | 337 | POSITIVE | COMPLETE |
| | 22 | 23741 . . . 24469 | 242 | POSITIVE | COMPLETE |
| | 23 | 24512 . . . 25228 | 238 | POSITIVE | COMPLETE |
| | 24 | 25239 . . . 26183 | 314 | POSITIVE | COMPLETE |
| | 25 | 26177 . . . 27013 | 278 | POSITIVE | COMPLETE |
| | 26 | 27010 . . . 28200 | 396 | POSITIVE | COMPLETE |
| | 27 | 28197 . . . 29168 | 323 | POSITIVE | COMPLETE |
| | 28 | 29168 . . . 29962 | 264 | POSITIVE | COMPLETE |
| | 29 | 30003 . . . 30980 | 325 | POSITIVE | COMPLETE |
| | 30 | 30980 . . . 31942 | 320 | POSITIVE | COMPLETE |
| | 31 | 31981 . . . 32988 | 335 | POSITIVE | COMPLETE |
| | 32 | 32985 . . . 34013 | 342 | POSITIVE | COMPLETE |
| | 33 | 34813 . . . 34061 | 250 | NEGATIVE | COMPLETE |
| | 34 | 35012 . . . 35737 | 241 | POSITIVE | COMPLETE |
| | 35 | 35734 . . . 36678 | 314 | POSITIVE | COMPLETE |
| | 36 | 38312 . . . 36855 | 485 | NEGATIVE | COMPLETE |
| | 37 | 38516 . . . 39367 | 283 | POSITIVE | COMPLETE |
| | 38 | 39369 . . . 40415 | 348 | POSITIVE | COMPLETE |
| | 39 | 40636 . . . 41682 | 348 | POSITIVE | COMPLETE |
| | 40 | 41676 . . . 43076 | 466 | POSITIVE | COMPLETE |
| | 41 | 43081 . . . 44091 | 336 | POSITIVE | COMPLETE |
| | 42 | 44081 . . . 45055 | 324 | POSITIVE | COMPLETE |

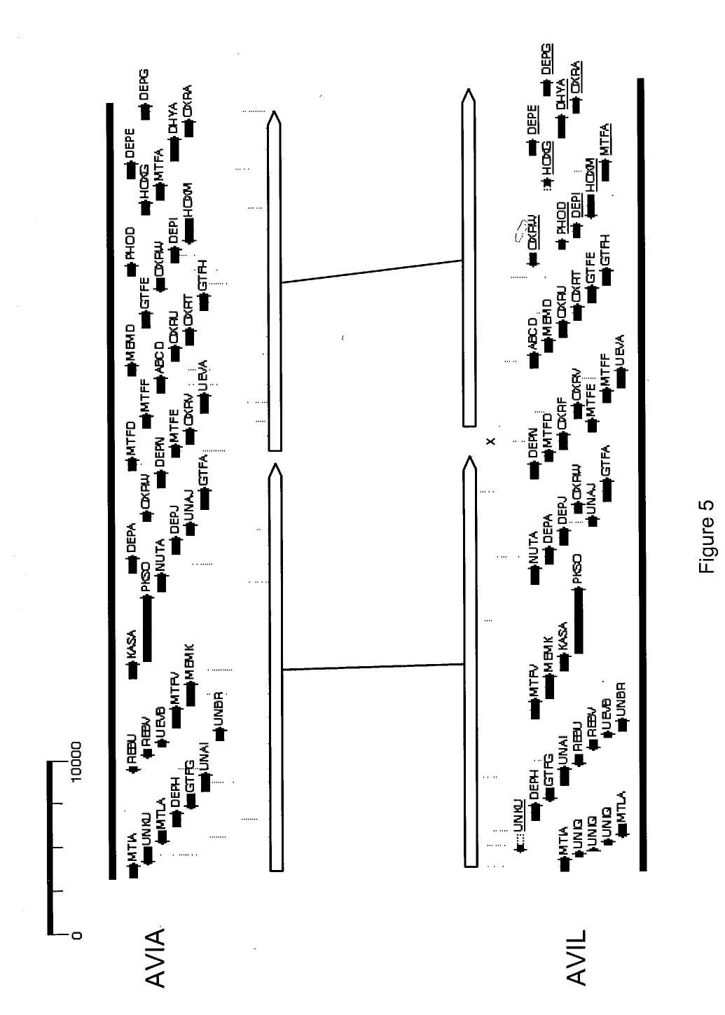

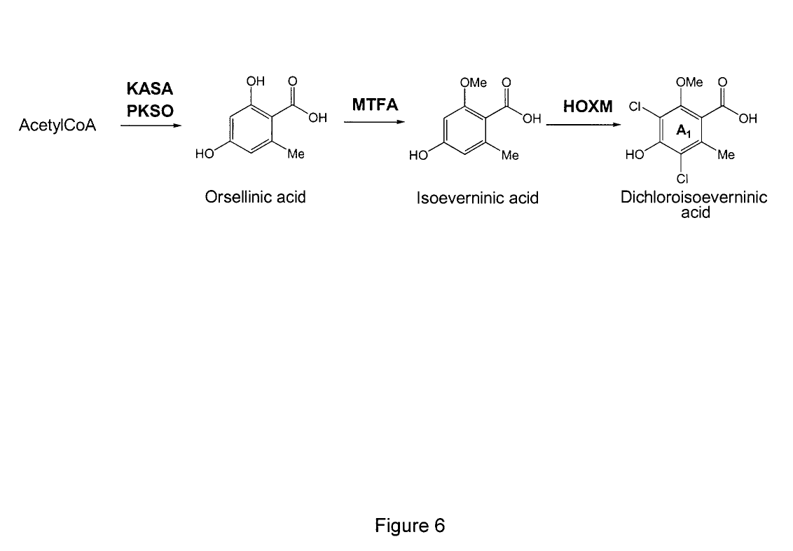

AVIA was compared to the avilamycin A locus of Streptomyces viridochromogenes Tu57 (herein referred to as AVIL), GenBank nucleotide accession AF333038, Weitnauer et al. 2001 Chemistry and Biology Vol. 8, pp. 569-581. FIG. 5 illustrates that the presence and orientation of homologous ORFs in AVIA and AVIL. The scale at the top of the FIG. 1 is in kilobasepairs. Solid black arrows depict the relative positions of the individual ORFs in AVIA and AVIL with the arrowhead indicating the orientation of each ORF; the corresponding four letter family designation is indicated to the right of each ORF. The empty arrows between the two loci highlight segments that contain a number of ORFs whose relative order and orientation is identical between the two loci. The order and orientation of ORFs in AVIA is identical to that in AVIL with the exception of one ORF in the middle of the AVIL locus designated as a member of the OXRF family of oxidoreductases. The ORF designated OXRF in AVIL does not have a counterpart in the AVIA locus (as indicated by the ‘X’). The ORFs in AVIL whose four-letter protein family designation is underlined are not disclosed in the Streptomyces viridochromogenes Tu57 avilamycin A biosynthetic gene cluster in the GenBank nucleotide accession AF333038. Using the compositions and methods of the present invention, we have now identified additional ORFs at the 3′ end of the AVIL locus. The sequence of the ORFs in AVIL corresponding to proteins considered designated HOXG and UNKU appear to be disrupted by frameshifts. It is unclear whether these frameshifts reflect real perturbations of the ORFs (rendering them inactive) or if they are due to sequencing errors. We have detected portions of the AVIL UNKU ORF in the region in which three small ORFs (designated UNIQ) had earlier been reported. We believe the presence of multiple frameshifts in the region corresponding to the UNKU ORF of AVIL may have resulted in the three earlier UNIQ ORFs report based on the wrong strand.

### EXAMPLE 3

### Genes Indicative of Orthosomycin Biosynthetic Loci

Certain genes in orthosomycin loci are associated with structural features that are common to all classes of orthosomycin oligosacharides and indicative of orthosomycin biosynthetic loci. Table IV lists the protein families and their respective ORF numbers in four orthosomycin loci, namely EVER (described in Example 1); AVIA (described in Example 2); EVEA (described in Example 10); and AVIL (described in Weitnauer et al. 2001 Chemistry and Biology Vol. 8, pp. 569-581). Each row in Table IV relates to a single protein family and identifies ORFs considered to be members of that protein family in the respective loci. The protein family is identified by its four-letter designation (see Table I). Thus, if a member of a particular protein family is found in one or more of EVEA, EVER, AVIA and AVIL those members will be listed in the same row. The symbols ## and ### and lower-case family designations for locus AVIL specify those ORFs that are not disclosed in the Streptomyces viridochromogens Tu57 amilamycin A locus in GenBank nucleotide accession AF333038 but that are now identified using the compositions and methods of the present invention. EVER and EVEA are examples of everninomicin-type orthosomycins; while AVIA and AVIL loci are examples of avilamycin-type orthosomycins.

The protein families in these four orthosomycin biosynthetic loci can be categorized into 5 groups based on their distribution: i) seventeen (17) families that are common among orthosomycin loci but also found in non-orthosomycin loci and therefore are not considered specific to orthosomycin; ii) seventeen (17) families that are common to most orthosomycin loci and are considered diagnostic of orthosomycin loci, as described in more detail below; iii) six (6) families that are diagnostic of avilamycin-type orthosomycin loci, particularly when found together with members of the protein families of group (ii) as described in more detail in Example 5; iv) nine (9) families that are considered diagnostic of everninomicin-type orthosomycin loci, particularly when found together with members of the protein families of group (ii), as described in more detail in Example 4; and v) a group of 12 miscellaneous families (not including those designated as ‘UNIQ’ in the AVIL locus) that are not present in all four orthosomycin loci and/or not unique to orthosomycin loci. Using the compositions and methods of the invention, the region of the strand opposite AVIL ORFs 2, 3, and 4 as disclosed in Weitnauer et al. 2001 Chemistry and Biology Vol. 8, pp. 569-581 exhibits homology to the AVIA member of protein family UNKU. Accordingly, it is believed that AVIL ORFs 2, 3, and 4 as disclosed in Weitnauer et al. may be incorrect conceptual translations and are designated as UNIQ in Table IV.

TABLE IV

| EVEA | | EVER | | AVIA | | AVIL | |
|---|---|---|---|---|---|---|---|
| ORF # | FAMILY | ORF # | FAMILY | ORF # | FAMILY | ORF # | FAMILY |

i) ORFs not necessarily unique to, but common to orthosomycin loci

| | | | | | | | |
|---|---|---|---|---|---|---|---|
| 3 | DEPA | 29 | DEPA | 16 | DEPA | 18 | DEPA |
| 22 | DEPE | 39 | DEPE | 39 | DEPE | ### | depe |
| 25 | DEPG | 10 | DEPG | 42 | DEPG | ### | depg |
| 34 | DEPH | 41 | DEPH | 4 | DEPH | 6 | DEPH |
| 28 | DEPI | 34 | DEPI | 35 | DEPI | ### | depi |
| 4 | DEPJ | 30 | DEPJ | 17 | DEPJ | 19 | DEPJ |
| 23 | DHYA | 38 | DHYA | 40 | DHYA | ### | dhya |
| 7 | GTFA | 16 | GTFA | 20 | GTFA | 22 | GTFA |
| 20 | HOXM | 20 | HOXM | 36 | HOXM | ### | hoxm |
| 17 | KASA | 14 | KASA | 13 | KASA | 15 | KASA |
| 15 | MEMK | 2 | MEMK | 12 | MEMK | 14 | MEMK |
| 44 | MTFA | 11 | MTFA | 38 | MTFA | ### | mtfa |
| 13 | MTFV | 6 | MTFV | 11 | MTFV | 13 | MTFV |
| 2 | NUTA | 28 | NUTA | 15 | NUTA | 17 | NUTA |
| 24 | OXRA | 37 | OXRA | 41 | OXRA | ### | oxra |
| 16 | PKSO | 32 | PKSO | 14 | PKSO | 16 | PKSO |
| 26 | REBV | 36 | REBV | 8 | REBV | 10 | REBV |

ii) ORFS which are diagnostic of orthosomycin loci

| | | | | | | | |
|---|---|---|---|---|---|---|---|
| 33 | GTFE | 24 | GTFE | 31 | GTFE | 34 | GTFE |
| 27 | GTFG | 35 | GTFG | 5 | GTFG | 7 | GTFG |
| 31 | GTFH | 8 | GTFH | 32 | GTFH | 35 | GTFH |
| 43 | HOXG | 12 | HOXG | 37 | HOXG | ## | hoxg |
| 8 | MTFD | 15 | MTFD | 22 | MTFD | 24 | MTFD |
| 10 | MTFE | 19 | MTFE | 23 | MTFE | 26 | MTFE |
| 12 | MTFF | 5 | MTFF | 25 | MTFF | 28 | MTFF |
| 45 | MTLA | 40 | MTLA | 3 | MTLA | 5 | MTLA |
| 11 | OXRV | 18 | OXRV | 24 | OXRV | 27 | OXRV |
| 30 | OXRW | 26 | OXRW | 33 | OXRW | ## | oxrw |
| 6 | OXRW | 31 | OXRX | 19 | OXRW | 21 | OXRW |
| 5 | UNAJ | | | 18 | UNAJ | 20 | UNAJ |
| 29 | PHOD | 33 | PHOD | 34 | PHOD | ## | phod |
| 14 | UEVA | 17 | UEVA | 26 | UEVA | 29 | UEVA |
| 32 | UNKU | 25 | UNKU | 2 | UNKU | ## | unku |
| | | 13 | MTIA | 1 | MTIA | 1 | MTIA |
| | | 9 | UEVB | 9 | UEVB | 11 | UEVB |

iii) ORFs not necessarily unique to, but common to avilamycin-type loci

| | | | |
|---|---|---|---|
| 27 | ABCD | 30 | ABCD |
| 21 | DEPN | 23 | DEPN |
| 28 | MEMD | 31 | MEMD |
| 6 | UNAI | 8 | UNAI |
| 7 | REBU | 9 | REBU |
| 10 | UNBR | 12 | UNBR |

iv) ORFs not necessarily unique to, but common to everninomicin-type loci

| | | | |
|---|---|---|---|
| 37 | DATC | 43 | DATC |
| 40 | DEPF | 46 | DEPF |
| 39 | EPIM | 45 | EPIM |
| 35 | GTFA | 21 | GTFA |
| 38 | MTFV | 44 | MTFV |
| 36 | OXBN | 42 | OXBN |
| 19 | OXCO | 4 | OXCO |
| 18 | MTFG | 3 | MTFG |
| 41 | UNBB | 47 | UNBB |

v) Miscellaneous ORFs present in various orthosomycin loci

| | | | | | | | |
|---|---|---|---|---|---|---|---|
| | | 23 | OXRT | 30 | OXRT | 33 | OXRT |
| | | 22 | OXRU | 29 | OXRU | 32 | OXRU |
| 9 | OXRF | | | | | 25 | OXRF |
| 42 | OXRF | | | | | | |
| 21 | EFFA | | | | | | |
| 1 | HYDH | | | | | | |
| | | 27 | DEPD | | | | |
| | | 48 | ENGA | | | | |
| | | 50 | KINB | | | | |
| | | 1 | MTBA | | | | |
| | | 7 | MTFH | | | | |
| | | 49 | REGL | | | | |
| | | | | | | 2 | UNIQ |
| | | | | | | 3 | UNIQ |
| | | | | | | 4 | UNIQ |

Group (ii) of Table IV, represent seventeen (17) protein families considered diagnostic of orthosomycin loci, namely GTFE, GTFG, GTFH, HOXG, MTFD, MTFE, MTFF, MTLA, OXRV, OXRW, OXRW, UNAJ, PHOD, UEVA, UNKU, UEVB, and MTIA. The 17 protein families includes two families designated OXRW, although in EVER one of the OXRW proteins is fused with a member of the UNAJ protein family and is therefore designated OXRX. Hence, EVER contains a single freestanding member of OXRW and contains no freestanding member of UNAJ. The UEVB, and MTIA families are not present in the EVEA locus, but are nonetheless considered to be diagnostic of orthosomycin loci as they are found in the other three orthosomycin loci and no known homologues have been described elsewhere to date. The seventeen protein families that are considered diagnostic of orthosomycin loci are those families for which no homologues exist that are naturally involved in the biosynthesis of compounds other than orthosomycins and/or no homologues exist that are in a context other than an orthosomycin biosynthetic locus. However, an orthosomycin biosynthetic locus is not necessarily expected to include a member of each of the seventeen protein families considered diagnostic of orthosomycin loci.

The following members of the seventeen protein families considered diagnostic of orthosomycin biosynthetic loci are identified in EVEA, EVER, AVIA and AVIL: GTFE (AVIA ORF 31, SEQ ID NO: 51; AVIL accession no. AAK83192; EVER ORF 24, SEQ ID NO: 53; EVEA ORF 33, SEQ ID NO: 55); GTFG (AVIA ORF 5, SEQ ID NO: 57; AVIL accession no. AAK83170; EVER ORF 35, SEQ ID NO: 59; EVEA ORF 27, SEQ ID NO: 61); GTFH (AVIA ORF 32, SEQ ID NO: 63; AVIL accession no. AAK83193; EVER ORF 8, SEQ ID NO: 65; EVEA ORF 31, SEQ ID NO: 67); HOXG (AVIA ORF 37, SEQ ID NO: 69; EVER ORF 12, SEQ ID NO: 71; EVEA ORF 43; SEQ ID NO: 73); MTFD (AVIA ORF 22, SEQ ID NO: 99; AVIL accession no. AAK83184; EVER ORF 15, SEQ ID NO: 101; EVEA ORF 8, SEQ ID NO: 103), MTFE (AVIA ORF 23, SEQ ID NO: 105; AVIL accession no. AAK83186; EVER ORF 19, SEQ ID NO: 107; EVEA ORF 10, SEQ ID NO: 109), MTFF (AVIA ORF 25, SEQ ID NO: 111; AVIL accession no. AAK83188; EVER ORF 5, SEQ ID NO: 113; EVEA ORF 12, SEQ ID NO: 115); MTLA (AVIA ORF 3, SEQ ID NO: 127; AVIL accession no. AAG32067; EVER ORF 40, SEQ ID NO: 129; EVEA ORF 45, SEQ ID NO: 131); MTIA (AVIA ORF 1, SEQ ID NO: 123; AVIL accession no. AAG32066; EVER ORF 13, SEQ ID NO: 125); OXRV (AVIA ORF 24, SEQ ID NO: 153; AVIL accession no. AAK83187; EVER ORF 18, SEQ ID NO: 155; EVEA ORF 11, SEQ ID NO: 157); OXRW (AVIA ORF 33, SEQ ID NO: 159; EVER ORF 26, SEQ ID NO: 161; EVEA ORF 30, SEQ ID NO: 163); OXRW (AVIA ORF 19, SEQ ID NO: 167; EVEA ORF 6, SEQ ID NO: 173; AVIL accession no. AAK83181), in EVER the second member of the OXRW family is fused with a protein from the UNAJ family and the combined polypeptide is designated as OXRX (EVER ORF 31, SEQ ID NO: 169); PHOD (AVIA ORF 34, SEQ ID NO: 175; EVER ORF 33, SEQ ID NO: 177; EVEA ORF 29, SEQ ID NO: 179); UNAJ (AVIA ORF 18, SEQ ID NO: 165; EVEA ORF 5, SEQ ID NO: 171), in EVER the UNAJ protein is fused with the second member of the OXRW family and the combined polypeptide is designated as OXRX (EVER ORF 31, SEQ ID NO: 169); UEVA (AVIA ORF 26, SEQ ID NO: 193; AVIL accession no. AAK83189; EVER ORF 17, SEQ ID NO: 195; EVEA ORF 14, SEQ ID NO: 197); UEVB (AVIA ORF 9, SEQ ID NO: 199; AVIL accession no. AAK83174; EVER ORF 9, SEQ ID NO: 201; and UNKU (AVIA ORF 2, SEQ ID NO: 203; EVER ORF 25, SEQ ID NO: 205; EVEA ORF 32, SEQ ID NO: 207).

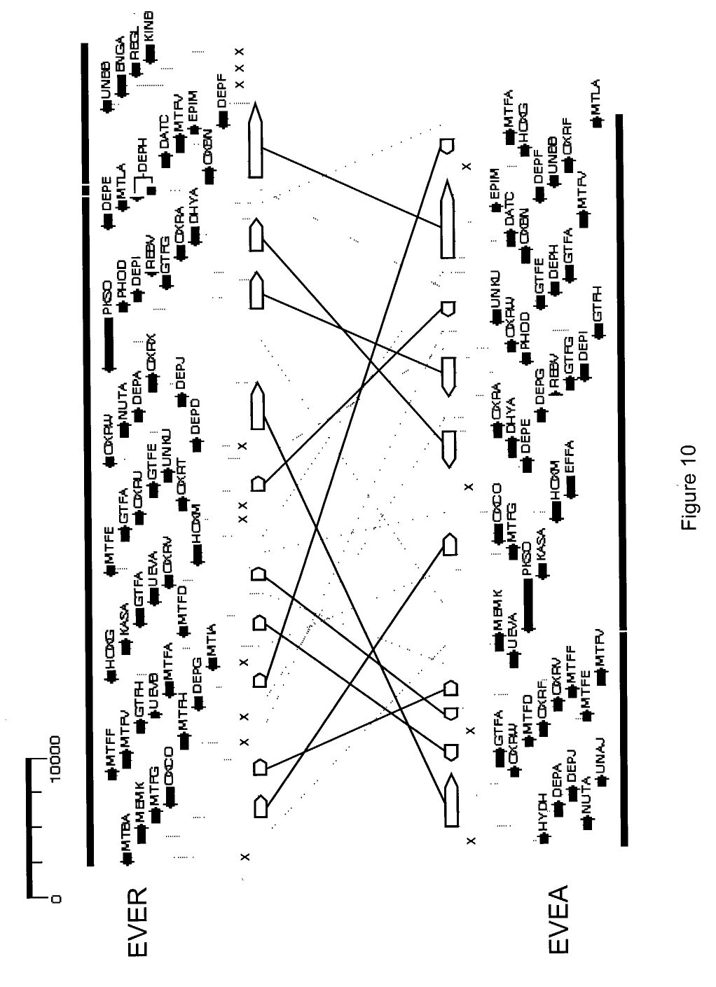

The homologues from the four orthosomycin loci belonging to each of the seventeen families diagnostic of orthosomycin loci were compared by BLAST. The percent identity and percent similarity of the amino acid sequences are reported in the sixteen tables identified as Tables V to XX. Values in Tables V to XX are expressed as % identity (%similarity) following a pairwise blast 2 sequences; n/a, comparison is not applicable since UNAJ and OXRW are non homologous ORFs; XXX, denotes that a family homolog is not present in the locus. AVIL ORFs with an asterisk are present in the publicly available nucleotide sequence of the avilamycin locus (as shown in FIG. 10) but were not submitted to the GenBank protein database; homology values listed for such ORFs were obtained with tblastn using the default settings and the corresponding AVIA homologues as queries. “Refer to figure” denotes those avilamycin ORFs which are segmented, presumably because of frameshifts in the publicly available sequence, see the corresponding TBLASTN alignments below.

TABLE V
Homology among the GTFE family members

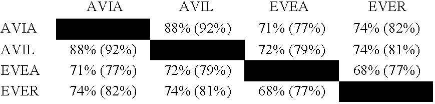

TABLE VI
Homology among the GTFG family members

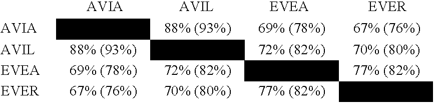

TABLE VII
Homology among the GTFH family members

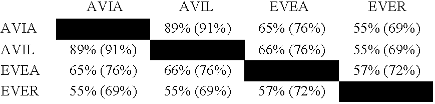

TABLE VIII
Homology among the HOXG family members

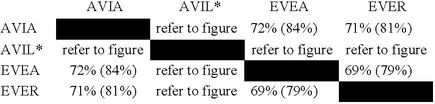

TABLE IX
Homology among the MTFD family members

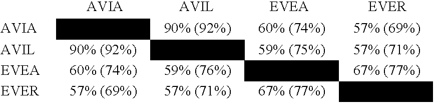

TABLE X
Homology among the MTFE family members

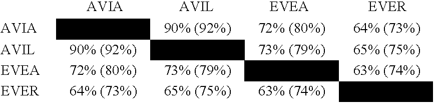

TABLE X1
Homology among the MTFF family members

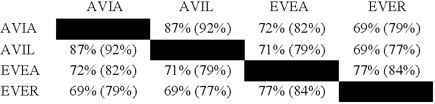

TABLE XII
Homology among the MTIA family members

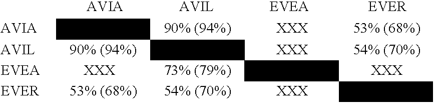

TABLE XIII
Homology among the MTLA family members

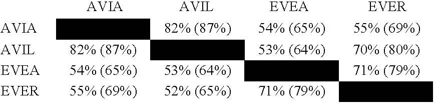

TABLE XIV
Homology among the OXRV family members

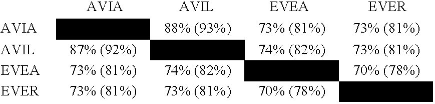

TABLE XV
Homology among the OXRW family members

| | AVIA | AVIL* | EVEA | EVER |
|---|---|---|---|---|
| AVIA | 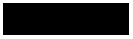 | 88% (91%) | 68% (75%) | 66% (76%) |
| AVIL* | 88% (91%) | 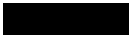 | 69% (76%) | 66% (77%) |
| EVEA | 68% (75%) | 69% (76%) | 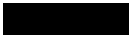 | 71% (80%) |
| EVER | 66% (76%) | 66% (77%) | 71% (80%) | 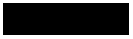 |

TABLE XVI
Homology among the PHOD family members

| | AVIA | AVIL* | EVEA | EVER |
|---|---|---|---|---|
| AVIA | 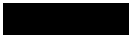 | 87% (90%) | 63% (71%) | 63% (71%) |
| AVIL* | 87% (90%) | 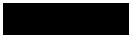 | 63% (70%) | 61% (69%) |
| EVEA | 63% (71%) | 63% (70%) | 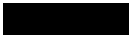 | 64% (75%) |
| EVER | 63% (71%) | 61% (69%) | 64% (75%) | 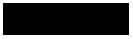 |

TABLE XVII
Homology among the UEVA family members

| | AVIA | AVIL | EVEA | EVER |
|---|---|---|---|---|
| AVIA | 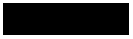 | 95% (96%) | 77% (83%) | 70% (79%) |
| AVIL | 95% (96%) | 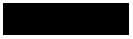 | 78% (84%) | 71% (79%) |
| EVEA | 77% (83%) | 78% (84%) | 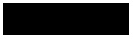 | 73% (82%) |
| EVER | 70% (79%) | 71% (79%) | 73% (82%) | 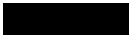 |

TABLE XVIII
Homology among the UEVB family members

| | AVIA | AVIL | EVEA | EVER |
|---|---|---|---|---|
| AVIA | 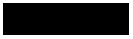 | 93% (98%) | XXX | 81% (94%) |
| AVIL | 93% (98%) | 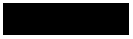 | XXX | 79% (95%) |
| EVEA | XXX | XXX | 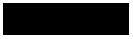 | XXX |
| EVER | 81% (94%) | 79% (95%) | XXX | 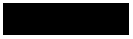 |

TABLE XIX
Homology among the UNKU family members

| | AVIA | AVIL* | EVEA | EVER |
|---|---|---|---|---|
| AVIA | 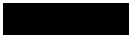 | 59% (63%) | 64% (75%) | 62% (73%) |
| AVIL* | refer to figure | 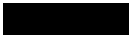 | refer to figure | refer to figure |
| EVEA | 64% (75%) | refer to figure | 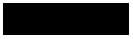 | 64% (75%) |
| EVER | 62% (73%) | refer to figure | 64% (75%) | 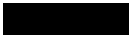 |

TABLE XX
Homology among the OXRX and UNAJ + OXRW family members

| | AVIA | | AVIL | | EVEA | | EVER |
|---|---|---|---|---|---|---|---|
| | OXRW | UNAJ | OXRW | UNAJ | OXRW | UNAJ | OXRX |
| AVIA_OXRW | 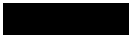 | n/a | 92% (92%) | n/a | 66% (75%) | n/a | 72% (78%) |
| AVIA_UNAJ | n/a | 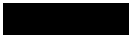 | n/a | 81% (87%) | n/a | 66% (75%) | 69% (78%) |
| AVIL_OXRW | 92% (92%) | n/a | 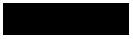 | n/a | 66% (75%) | n/a | 74% (81%) |
| AVIL_UNAJ | n/a | 81% (87%) | n/a | 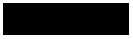 | n/a | 58% (68%) | 62% (71%) |
| EVEA_OXRW | 66% (75%) | n/a | 66% (75%) | n/a | 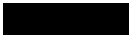 | n/a | 73% (80%) |
| EVEA_UNAJ | n/a | 66% (75%) | n/a | 58% (68%) | n/a | 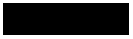 | 77% (83%) |
| EVER_OXRX | 72% (78%) | 69% (78%) | 74% (81%) | 62% (71%) | 73% (80%) | 77% (83%) | 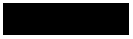 |

Without intending to be limited to any particular mechanism of action or biosynthetic scheme, the protein families which are found in all orthosomycin biosynthetic loci can explain formation of structural elements that define orthosomycin compounds. FIG. 2 shows one scheme for the biosynthesis of dichloroisoeverninic acid from acetyl CoA. In the scheme of FIG. 2, the KASA enzyme (a putative ketoacyl synthase) is a priming enzyme which loads acetyl CoA onto the PKSO (a putative orsellinic acid synthase). MFTA (similar to aromatic O-methyl transferases) methylates orsellinic acid, and HOXM (similar to non-heme hydroxylase/halogenases) chlorinates isoeverninic acid. Member of other protein families present in all orthosomycin loci may also be involved in the biosynthesis of dichloroisoeverninic acid moiety (or moieties) of orthosomycins.

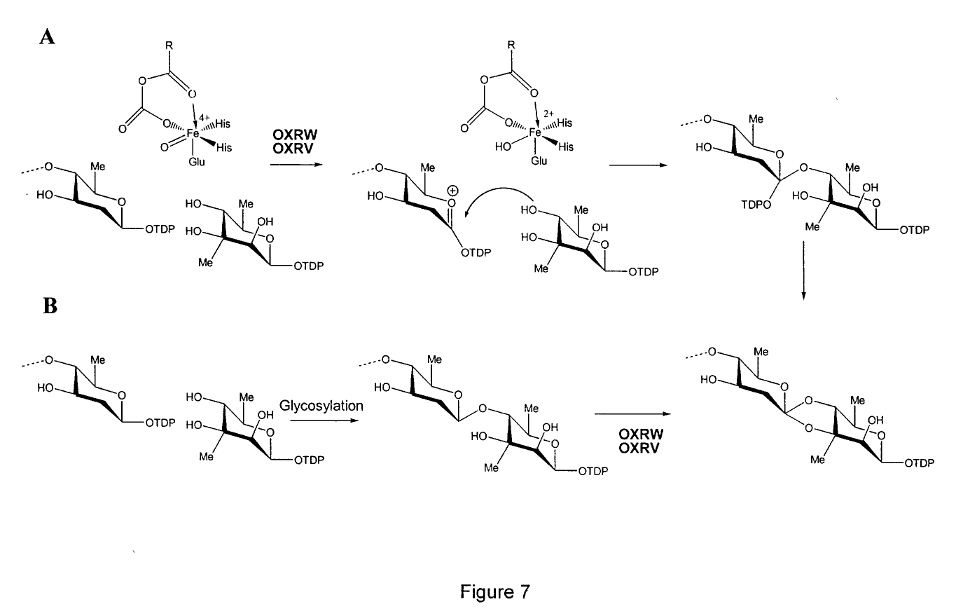

FIG. 7 shows two schemes (A and B) for orthoester formation by the two OXRW's and OXRV, all of which have sequence similarity to iron alpha-ketoglutaric acid dependent enzymes. Scheme A is distinguished from scheme B in that the former does not implicate the action of a glycosyltransferase enzyme prior to the oxidative C—O coupling reaction. Similar oxidative C—O coupling has been observed in other iron alpha-ketoglutaric acid dependent enzymes such as clavaminic acid synthase (Salowe SP, Marsh EN, Townsend, CA, Biochemistry 29(27): 6499-6508). Members of other protein families present in all orthosomycin loci may also be involved in the formation of the orthoester linkage(s) of orthosomycins.

### EXAMPLE 4

### Genes Specific to Everninomicin-Type Orthosomycin Biosynthetic Loci

Protein families DATC, DEPF, EPIM, GTFA, MTFG, MTFV, OXBN, OXCO, and UNBB (group (iv) of Table IV) are considered diagnostic of everninomicin-type orthosomycin biosynthetic loci and everninomicin-type orthosomycin producers, particularly when a member of at least one, preferably 2, more preferably 3, still more preferably 4, still more preferably 5 and most preferably 6 or more of the nine protein families is found together with a member of one, preferably 2, more preferably 3, still more preferably 4, still more preferably 6, and most preferably 8 or more members of the seventeen orthosomycin specific protein families listed in group (ii) of Table IV. DATC, DEPF, EPIM, GTFA, MTFV, OXBN, and OXCO are not unique to everninomicin-type orthosomycin loci as close relatives are associated with secondary metabolism unrelated to orthosomycin biosynthesis. MTFG and UNBB represent two families that are considered to be unique to everninomicin-type orthosomycin loci as no homologues exist that are naturally involved in the biosynthesis of compounds other than everninomicin-type orthosomycins and/or no homologues exist that are in a context other than an everninomicin-type orthosomycin biosynthetic locus. An everninomicin-type orthosomycin biosynthetic locus is not expected to necessarily contain a member of the nine protein families considered diagnostic of everninomicin-type orthosomycin loci.

Homologues of the nine protein families diagnostic of everninomicin-type orthosomycin loci and present in EVER and EVEA were compared by Blast analysis with the default parameters. The percent identity and percent similarity of the amino acid sequences are reported in Table XXI.

TABLE XXI

| | % identity | % similarity |
|---|---|---|
| DATC | 78% | 84% |
| DEPF | 64% | 74% |
| EPIM | 74% | 84% |
| GTFA | 60% | 74% |
| MTFG | 70% | 75% |
| MTFV | 81% | 85% |
| OXBN | 80% | 86% |
| OXCO | 77% | 85% |
| UNBB | 71% | 82% |

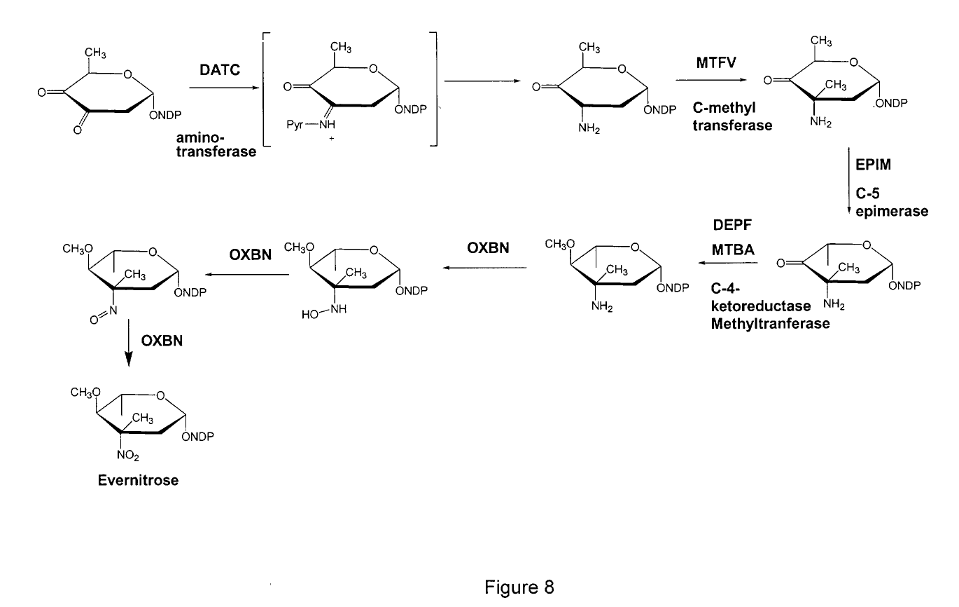

Without intending to be limited to any particular mechanism or biosynthetic scheme, the protein families diagnostic of everninomicin-type orthosomycin biosynthetic loci can explain formation of structural elements that characterize everninomicin compounds. FIG. 8 shows one route for the formation of the nitrosugar residue of everninomicin. In FIG. 8 the amine oxidation reactions are catalyzed sequentially by OXBN, with sequence similarity to flavin-dependent monooxygenases.

### EXAMPLE 5

### Genes Specific to Avilamycin-Type Orthosomycin Biosynthetic Loci

Protein families ABCD, DEPN, MEMD, REBU, UNAI and UNBR (group (iii) of Table IV) are considered to be diagnostic of avilamycin-type orthosomycin, particularly when a member of one, preferably 2, more preferably 3, still more preferably 4 or more of the six protein families diagnostic of an avilamycin-type orthosomycin biosynthetic locus is found together with a member of one, preferably two, more preferably 4, still more preferably 6, still more preferably 8, and most preferably 10 or more members of the seventeen orthosomycin specific protein families listed in group (ii) of Table IV.

The six protein families considered diagnostic of avilamycin-type orthosomycin biosynthetic are ABCD (AVIA ORF 27, SEQ ID NO: 245; AVIL accession no. AAG32068); DEPN (AVIA ORF 21, SEQ ID NO: 247; AVIL accession no. AAK83183); MEMD (AVIA ORF 28, SEQ ID NO: 249; AVIL accession no. AAG32069); REBU (AVIA ORF 7, SEQ ID NO: 251; AVIL accession no. AAK83172); UNAI (AVIA ORF 6, SEQ ID NO: 253; AVIL accession no. AAK83171) and UNBR (AVIA ORF 10, SEQ ID NO: 255; AVIL accession no. AAK83175). ABCD, DEPN, MEMD, and UNAI are not unique to avilamycin-type orthosomycin loci as close relatives of their protein families exist in secondary metabolism unrelated to orthosomycin biosynthesis. REBU and UNBR members represent two families that are considered to be unique to avilamycin-type orthosomycin loci as no homologues exist that are naturally involved in the biosynthesis of compounds other than avilamycin-type orthosomycins and/or no homologues exist that are in a context other than an avilamycin-type orthosomycin biosynthetic locus. An avilamycin-type orthosomycin is not expected to necessarily include a member of each of the six protein families considered diagnostic of orthosomycin loci.

Homologues of the six families diagnostic of avilamycin-type orthosomycin loci and present in AVIA and AVIL were compared by Blast analysis. The percent identity and percent similarity of the amino acid sequences are reported in Table XXII.

TABLE XXII

| | % identity | % similarity |
|---|---|---|
| ABCD | 89% | 93% |
| DEPN | 89% | 94% |
| MEMD | 94% | 95% |
| REBU | 92% | 93% |
| UNAI | 86% | 92% |
| UNBR | 90% | 94% |

AVIA and AVIL both contain a two-component transport system that is not found in everninomicin-type loci. The ABCD and MEMD proteins in AVIA have been described as an ATP-binding transporter (AviABCI) and a transmembrane transporter (AviABCII), respectively, and are involved in conferring resistance of S. viridochromogenes to avilamycin A (Weitnauer et al., 2001, Antimicrob. Agents Chemother., Vol. 45, pp. 690-695). Based on the high sequence homology, corresponding ORF 27 (SEQ ID NO: 245) and ORF 28 (SEQ ID NO: 249) in the AVIA are believed to carry out analogous functions. These proteins are also similar to the DrrA and DrrB proteins of S. peucetius involved in conferring resistance of that organism to daunorubicin and doxorubicin. The ABCD protein, the AviABCI protein and the DrrA proteins are similar to proteins encoded by the mdr genes of mammalian tumor cells, which confer resistance on these cells to many structurally unrelated chemotherapeutic agents. ABCD and MEMD act jointly to confer resistance to avilamycin-type orthosomycin oligosaccharides by a mechanism analogous to the antiport mechanism established for mammalian tumor cells that contain amplified or overexpressed mdr genes (Guilfoile et al., 1991, Proc. Natl. Acad. Sci. USA, Vol. 88, pp. 8553-8557). AVIA and AVIL both contain a dehydratase/epimerase that is designated as ‘DEPN’ and which is distinct from the dehydratase/epimerase enzymes in the everninomicin-type orthosomycin loci. AVIA and AVIL both contain an ORF of unknown function designated as ‘UNAI’ for which no homologue is present in the everninomicin-type orthosomycin loci, but for which at least one homologue exists, hypothetical protein SCF55.28c of Streptomyces coelicolor A3(2)

### EXAMPLE 6

### Design of Diagnostic Nucleic Acid Sequences for Identifying Orthosomycin Genes by Hybridization or by PCR Amplification

Three of the seventeen families of proteins common to orthosomycin oligosaccharide biosynthetic loci were used to design oligonucleotides that may be used either as hybridization probes or as PCR primers for the purpose of identifying orthosomycin biosynthetic loci in other organisms. The three families of proteins that were used in this example include UEVA, UEVB, and HOXG. The nucleotide sequences of the UEVA, UEVB, and HOXG protein families from EVER, namely EVER ORFs 17, 9, and 12 (SEQ ID NOS: 195, 201 and 71 respectively), and from AVIA, namely AVIA ORFs 26, 9, and 37 (SEQ ID NOS: 193, 199 and 69 respectively) were aligned by pairwise comparison using ‘BLAST 2 Sequences’, a BLAST-based tool for aligning two protein or nucleotide sequences (Tatiana et al. 1999 FEMS Microbiol Lett. 174:247-250). Parameters were all default settings except that filtering (masking of segments of the query sequence that have low compositional complexity) was not applied.

The alignments of the EVER and AVIL sequences for their UEVA, UEVB and HOXG proteins are shown below in Tables XXIII, XXIV and XXV respectively. Table XXIII is a nucleic acid alignment of the UEVA protein family, comparing AVIA ORF 26 (SEQ ID NO: 193) and EVER ORF 17 (SEQ ID NO: 195). Table XXIV is a nucleic acid alignment of the UEVB protein family, comparing AVIA ORF 9 (SEQ ID NO: 199) and EVER ORF 9 (SEQ ID NO: 201). Table XXV is a nucleic acid alignment of the HOXG protein family, comparing AVIA ORF 37 (SEQ ID NO: 69) and EVER ORF 12 (SEQ ID NO: 71). Several well-conserved regions of the alignment that served as a basis for designing diagnostic oligonucleotides are highlighted (‘>’ is used to indicate oligonucleotides oriented in the ‘sense’ direction; ‘<’ is used to indicate oligonucleotides oriented in the ‘antisense’ direction; and ‘\hat{0}’ is used to indicate a control oligonucleotide that has the same sequence as one strand but with inverted polarity and hence is unable to hybridize to either strand, thus serving as a negative control).

TABLE XXIII

| | | UEVA-S1 | |
|---|---|---|---|
| | | >>>>>>>>>>>>>>>>>>>>>>>>>> | |
| AVIA_ORF26: | 30 | gtgcgtgctgccgtggatccacatgtgcgcctccatcgacggcgtctacggccggtgctg | 89 |
| | | \|\|\| \|\|\|\|\|\|\|\|\|\|\|\|\|\|\|\|\|\| \| \|\|\|\|\|\|\|\|\|\|\|\|\|\|\|\|\|\|\|\|\|\|\|\|\|\|\|\|\|\|\|\|\|\|\| | |
| EVER_ORF17: | 57 | gtgtgtgctgccgtggatccacctctgcgcctccatcgacggcgtctacggccggtgctg | 116 |
| | | > | |
| AVIA_ORF26: | 90 | cgtggacgactccatgtaccacaacgagctgtacgagtccgtggacgagccggtcttcaa | 149 |
| | | \|\|\| \|\|\|\|\|\|\|\| \|\|\|\|\|\|\|\|\|\| \|\|\|\|\|\|\|\|\|\|\| \|\|\| \|\|\|\|\|\|\| \|\|\| | |
| EVER_ORF17: | 117 | cgtcgacgactcgatgtaccacacggagctgtacgacgagcaggaggagccggcgttcgc | 176 |
| | | UEVA-S2 | |
| | | >>>>>>>>>>>>>>>>>>>>>>>>>> | |
| AVIA_ORF26: | 150 | gctcaacgccgacgccgtcggctgcgcgcccaactcccgctacgccaaggacaacccgga | 209 |
| EVER_ORF17: | 177 | gctgaacgacgacgcgatcggttgctccccgggctcgcggtacgccaaggacaacccgga | 236 |
| | | > | |
| AVIA_ORF26: | 210 | cgaggtacgcgggctgacggaggcgttcaacagccccaacatgcggcgcacccggctgaa | 269 |
| | | \| \|\| \|\| \| \|\|\|\|\|\| \|\|\|\|\|\|\|\|\|\|\|\|\|\|\|\|\|\| \|\|\| \|\|\|\|\|\|\|\|\| | |
| EVER_ORF17: | 237 | ccgcgtgatgggcatccgggaggccttcaacagccccaacatgaagcggacccggctggc | 296 |
| AVIA_ORF26: | 270 | gatgctggccggcgagcgggtgtccgcgtgcgactactgctaccaccgcgaggaccgggg | 329 |
| | | \|\|\|\|\|\| \| \|\|\|\|\|\|\|\| \|\|\| \|\|\|\|\|\| \| \|\|\|\|\|\|\|\|\| \|\|\| \|\|\|\|\|\|\| \|\| | |
| EVER_ORF17: | 297 | gatgctcggtggcgagcgcgtggaggcgtgcaagtactgctacttccgggaggaccacgg | 356 |
| AVIA_ORF26: | 330 | cgcgacctcgtaccggcagagcatcaacgagcggttcgccgacacggtggacttcgccga | 389 |
| | | \|\|\| \|\| \|\|\|\|\|\|\|\|\|\| \| \|\|\|\|\| \|\|\|\|\|\| \| \| \| \|\|\| \|\|\| \| | |
| EVER_ORF17: | 357 | cgcccagtcctaccggcagaacgtcaaccgccggttccaccaggagtacgacctcgatgc | 416 |
| AVIA_ORF26 | 390 | cctggccgaacggaccgcccccgacggctcgttcgacgagttcccgttcttcctggacat | 449 |
| | | \|\| \|\|\|\| \|\| \|\|\|\|\|\|\|\| \| \|\|\|\|\|\| \|\| \|\|\|\| \|\|\|\|\|\|\|\|\|\|\|\| \|\| \|\|\|\|\| | |
| EVER_ORF17: | 417 | gcccgccgcccgtaccgccgcggacggcacggtcgaggagttcccgttctttctcgacat | 476 |
| AVIA_ORF26: | 450 | ccggttcggcaacacctgcaacctgcggtgcgtgatgtgcgcctacccggtcagctccgg | 509 |
| | | \| \|\|\|\|\|\|\|\|\|\|\| \|\|\|\|\|\|\|\|\|\|\|\|\|\|\|\|\|\| \|\|\|\|\|\| \|\|\|\|\|\|\|\|\|\| \|\| \|\|\| | |
| EVER_ORF17: | 477 | caggttcggcaacctctgcaacctgcggtgcgtcatgtgcacctacccggtgagttcctc | 536 |
| | | UEVA-AS1 | |
| | | <<<<<<<<<<<<<<<<<<<<<<<<<<< | |
| AVIA_ORF26: | 510 | ctggggcgccaagaagcggccgtcgtggtcgtccgcggtgatcgacccgtaccgcgagga | 569 |
| | | \|\|\|\|\|\|\|\|\|\|\|\|\| \| \|\| \|\|\|\|\|\|\|\|\|\|\|\|\|\|\|\|\|\|\|\| \|\|\|\|\|\|\|\|\|\|\|\|\|\|\|\|\| \|\| | |
| EVER_ORF17: | 537 | ctggggcgccaagcaacgcccgtcgtggtcgtccgcggtcatcgacccgtaccgcgacga | 596 |
| AVIA_ORF26: | 570 | cgaggagctgtgggcgacgctccgcgagaacgcccacctcatccgccggctgtacttcgc | 629 |
| | | \|\|\| \|\|\| \|\|\|\|\|\|\|\|\|\|\|\|\| \|\| \|\|\|\|\| \|\| \|\|\|\|\| \|\|\|\|\|\| \|\|\|\|\|\|\|\|\|\|\|\| | |
| EVER_ORF17: | 597 | cgacgagttgtgggcgacgctgcgggagaatgcgcacctgatccgcaagctgtacttcgc | 656 |
| AVIA_ORF26: | 630 | cggcggtgaaccgttcatgcagccgggccacttcgcgatgctcgacctgctgatcgagac | 689 |
| | | \|\|\|\|\| \|\|\|\|\| \|\|\| \|\|\|\| \|\|\|\|\| \|\| \|\|\|\|\| \|\|\|\|\|\|\|\| \|\|\|\|\| \| \|\| \|\| | |
| EVER_ORF17: | 657 | gggcggcgaacccttcctgcaaccgggtcatttcgccatgctcgagctgctcgtggaaac | 716 |
| AVIA_ORF26: | 690 | cggcaacgcgggcaacgtcgacatcgtctacaactccaacctcacggtgctcccggagaa | 749 |
| | | \|\|\| \|\|\|\|\|\| \|\|\|\|\|\|\|\|\|\|\|\|\| \|\|\|\|\|\|\|\| \|\|\|\|\| \|\| \|\| \|\|\|\|\|\| \|\| | |
| EVER_ORF17: | 717 | cgggaacgcgcacaacgtcgacatccagtacaactcgaacctgaccgtctccccggacaa | 776 |
| | | UEVA-AS2 | |
| | | <<<<<<<<<<<<<< | |
| AVIA_ORF26: | 750 | ggtcttcgaccgcttcccgcacttcaagagcgtcgggatcggcgcctcctgcgacggcgt | 809 |
| | | \| \| \| \| \| \| \| \|\|\|\|\|\|\|\|\|\|\|\|\|\|\| \|\| \|\|\|\|\| \|\| \|\|\|\|\|\|\|\|\|\|\|\|\|\| | |
| EVER_ORF17: | 777 | cgcgataaagctcctacggcacttcaagagcgtgggcatcggggcttcctgcgacggcgt | 836 |
| | | UEVA-AS2 | |
| AVIA_ORF26: | 810 | cggcgaggtcttcgagcgcatccggcagcccgcgaaatgggacgtgttcgtcgccaacgt | 869 |
| | | \|\|\|\|\|\|\|\|\| \|\|\|\|\| \|\|\|\|\|\|\| \|\| \|\|\| \|\|\|\| \| \|\|\|\|\| \|\|\|\|\| \| | |
| EVER_ORF17: | 837 | cggcgaggtgttcgaatacatccgggccggcgggaagtgggcggacttcgtggccaatct | 896 |
| AVIA_ORF26: | 870 | ccgccgggccaagaccgaggtgaacctctggctccaggtcgcgccccagcggctcaacct | 929 |
| | | \|\|\|\| \| \| \| \|\|\|\| \| \|\| \|\|\|\|\|\|\|\|\|\|\|\|\| \| \|\| \|\|\|\|\|\|\| \|\|\|\|\|\| | |
| EVER_ORF17: | 897 | gcgcctgctccggtccgacttcgacgtctggctccaggtgtccccgcagcggcacaacct | 956 |
| AVIA_ORF26: | 930 | gtgggggctgcgggacctgctgcacttcggccgcgaggagggcctcgacgcggacctcgc | 989 |
| | | \|\|\|\|\| \|\|\|\|\| \|\| \|\|\|\| \| \|\|\|\|\| \|\| \|\|\|\|\| \|\| \|\| \| \|\|\|\|\|\| \|\| | |
| EVER_ORF17: | 957 | gtgggacctgcgcaacgtgctcgagttcgcgcgtaccgaggggctggaggtggacctggc | 1016 |
| AVIA_ORF26: | 990 | caacgtcgtgcagtggcccgacgactactccgtcgccaacctcccggacgaggagaagcg | 1049 |
| | | \|\|\|\|\|\| \|\|\|\|\|\|\|\|\|\|\| \| \|\| \|\|\| \|\|\|\|\|\|\| \|\|\| \|\|\| \|\|\|\|\|\|\|\|\|\|\|\| | |
| EVER_ORF17: | 1017 | caacgtggtgcagtggccgcaggatctctcggtcgccagcctgtcggccgaggagaaggc | 1076 |
| AVIA_ORF26: | 1050 | gcgggcgaccgtcgagctggccgacctggccgagtggtgcgacagcctggactgggccaa | 1109 |
| | | \|\|\| \|\| \|\|\| \|\|\|\|\|\| \| \|\|\|\|\|\| \|\| \|\|\|\|\|\|\| \| \|\| \| \|\|\|\|\| \|\|\| | |
| EVER_ORF17: | 1077 | gcgcgccacccaggagctgacggacctgatcgcctggtgcgccgagctcggctgggccaa | 1136 |
| AVIA_ORF26: | 1110 | gcccgc 1115 | |
| | | \|\|\|\|\|\| | |
| EVER_ORF17: | 1137 | gcccgc 1142 | |

Identities = 840/1066 (77%)

TABLE XXIV

| | | | |
|---|---|---|---|
| AVIA_ORF 9: | 2 | tgaaaatcgaggtgctccaaccgacctgcaacctggacacggtgcgggacggtcgcggcg | 61 |
| | | \|\|\|\| \|\|\|\|\|\|\|\| \|\| \|\| \|\|\|\| \|\|\|\|\|\|\|\|\|\|\|\|\|\|\| \|\| \|\|\|\|\|\|\|\| \|\| \|\|\|\| | |
| EVER_ORF 9: | 2 | tgaagatcgaggtcctgcagccgagctgcaacctggacaccgtccgggacggccggggcg | 61 |
| AVIA_ORF 9: | 62 | gaattttcacctgggttcccccggagcccatcctggaattcaatatgctgcacctgtacc | 121 |
| | | \| \|\| \|\|\|\|\|\|\|\|\|\|\| \|\| \|\| \|\|\|\|\| \|\|\|\|\|\|\|\| \|\|\|\|\| \| \| \| \|\| \|\|\| | |
| EVER_ORF 9: | 62 | gcatcttcacctgggtgccaccagagccgatcctggagttcaacctcatcaccatgcacc | 121 |
| | | UEVB-S1 | |
| | | >>>>>>>>>>>>>>>>>>>>>>> | |
| AVIA_ORF 9: | 122 | cgggaaaggtgcgcggtctgcactaccacccgcacttcgtcgaatacctgctcttcgtcg | 181 |
| | | \| \|\| \|\|\|\|\| \|\| \|\| \|\|\|\|\|\|\|\|\|\|\|\|\|\|\|\|\|\|\|\|\|\|\| \|\|\|\|\|\|\|\|\|\|\| \|\|\|\|\|\|\| | |
| EVER_ORF 9: | 122 | ccggcaaggtccgtgggctgcactaccacccgcacttcgtggaatacctgctgttcgtcg | 181 |
| AVIA_ORF 9: | 182 | agggctcgggcgtgctggtcaccaaggacgacgccgacgacccgaactgcgaggaagagt | 241 |
| | | \| \|\| \|\|\| \|\|\|\|\|\|\|\| \|\|\|\|\|\|\|\|\|\|\| \| \|\|\|\|\|\|\|\| \|\|\|\|\| \|\|\| \|\|\|\| | |
| EVER_ORF 9: | 182 | acggggagggggtgctggtgaccaaggacgatccggacgaccccgactgcccggaggagt | 241 |
| AVIA_ORF 9: | 242 | tcatccacgtctcgcgcggcatctgcaccaggacgcccgcggggatcatgcacgccgtcc | 301 |
| | | \|\|\|\|\|\|\|\|\|\|\| \| \|\| \|\| \| \|\| \|\| \| \|\|\|\|\|\| \| \|\| \| \|\|\|\|\|\|\|\| \|\|\| | |
| EVER_ORF 9: | 242 | tcatccacgtcgcccgggggacgtgtacgcgcacgccctccggagtgatgcacgcggtct | 301 |
| AVIA_ORF 9: | 302 | acgccatcacgccgctgacgttcatcgccatgctcaccaagccctgggacgagtgcgacc | 361 |
| | | \|\| \| \|\|\|\|\|\| \|\|\|\|\| \| \|\|\| \| \|\|\|\|\|\| \| \|\|\| \|\| \|\|\|\|\|\|\|\|\|\|\| \|\| \| | |
| EVER_ORF 9: | 302 | actcgatcacgtcgctgtccttcgtggccatgttgacccgaccgtgggacgagtgtgatc | 361 |
| AVIA_ORF 9: | 362 | cgccgctggtccaggtcgagccgctgccgcacaccct 398 | |
| | | \|\|\|\| \| \|\|\|\|\|\|\|\| \|\|\|\|\|\|\|\|\|\|\|\|\|\|\|\|\|\|\| | |
| EVER_ORF 9: | 362 | cgcccatcgtccaggtgcagccgctgccgcacaccct 398 | |
| | | \hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ }\hat{ } | |
| | | UEVB_CTL1 | |

Identities = 314/397 (79%)

TABLE XXV

| | | | |
|---|---|---|---|
| AVIA_ORF37: | 16 | ctgaccgag--gagcaggtcgagggcttcgtctccgacggcttcgtccacctgccgggtg | 73 |
| | | \|\|\|\|\| \|\| \|\|\|\|\|\| \|\|\|\|\| \|\|\|\|\|\|\|\| \|\|\|\|\|\|\|\|\|\|\|\|\|\|\| \|\|\|\|\| \| | |
| EVER_ORF12: | 4 | ctgac--agccgagcagatcgagagcttcgtcgccgacggcttcgtccgggtgccgadcg | 61 |
| AVIA_ORF37: | 74 | cgttcccgggggagctcgccgaggaggcgcgcgcc--ctgctgtggcggcagctggacat | 131 |
| | | \| \|\|\|\|\| \| \| \|\|\|\|\|\|\|\| \|\|\| \|\| \|\|\| \|\|\|\|\| \|\|\| \|\|\| \|\| \|\|\| \| | |
| EVER_ORF12: | 62 | ctttccccgccgcgctcgccgccgagt-gc-cgcaatctgctctggaagcaactcgacgt | 119 |
| AVIA_ORF37: | 132 | gga-cccggacgac--c-cgggcacctggacgc-gggaggtggtccggctcggggtgcgc | 186 |
| | | \|\|\| \|\|\|\| \|\|\|\|\| \| \|\| \|\|\|\|\|\|\|\| \| \|\|\|\|\|\|\| \|\|\|\|\|\|\|\|\|\|\| \|\|\|\| | |
| EVER_ORF12: | 120 | ggatcccg-acgacagctcg---acctggac-cagggaggtcgtccggctcggtctgcgg | 174 |
| AVIA_ORF37: | 187 | gacgacgacgtgttcgtcc-gtgccgccaacaccccg-c--tgct-gcacgccgcctacg | 241 |
| | | \| \|\|\|\|\|\|\|\| \|\|\|\|\|\| \| \| \|\| \|\|\|\|\|\|\|\|\|\|\|\| \| \|\| \| \| \| \|\| \| \|\|\|\| | |
| EVER_ORF12: | 175 | ggcgacgacgcgttcgtgcagagc-gccaacaccccggcgttggtcg-aggc-g--tacg | 229 |
| AVIA_ORF37: | 242 | accagctcgccggggagggccgctggc-agccgctg-accca-ggtcggcacgttcccgg | 298 |
| | | \|\|\|\|\|\|\|\|\| \|\|\| \| \|\|\|\|\|\| \|\|\|\| \| \|\|\|\|\|\| \|\| \| \|\|\|\|\|\| \|\|\|\|\|\|\|\|\| | |
| EVER_ORF12: | 230 | accagctcgtcggtgcgggccggtggcga-ccgctggac--atggtcgggacgttcccga | 286 |
| | | HOXG-S1 | |
| | | >>>>>>>>>>>>>>>>>> | |
| AVIA_ORF37: | 299 | tgcggttccccgtg-acgaagcgg--ccggaggagaccgaggactacggctggcacatcg | 355 |
| | | \| \|\| \|\|\|\|\| \|\|\| \|\| \| \|\| \|\|\|\|\| \|\| \|\|\|\|\|\|\|\|\|\|\|\|\|\|\|\|\|\|\|\|\|\|\|\| | |
| EVER_ORF12: | 287 | tccgtttcccggtggacc--g-ggatccggaacaggccgaggactacggctggcacatcg | 343 |
| | | HOXG-S1 | |
| | | >>>>>>>>> | |
| AVIA_ORF37: | 356 | acgccagcttcctc-gcc--gagggcgccgacgcc--ga-c--cg--c--gactggtccg | 403 |
| | | \|\|\|\|\|\|\|\|\|\|\|\|\|\| \|\|\| \|\|\|\|\|\|\| \| \|\|\|\| \|\| \| \|\| \| \|\|\|\|\|\| \| | |
| EVER_ORF12: | 344 | acgccagcttcctcagccccgagggcgtggccgccatgagcagcggccaggactgggagg | 403 |
| AVIA_ORF37: | 404 | gcgagctcgacg-t-gatcccgccggactacgacaagatcttccggta-caacgtgtg-g | 459 |
| | | \|\|\|\|\|\|\|\| \|\| \| \| \| \|\|\|\|\|\|\|\|\|\|\|\|\|\|\| \|\|\|\|\|\|\|\| \| \| \|\|\|\| \|\| \| | |
| EVER_ORF12: | 404 | gcgagctcc-cgctcg-tgccgccggactacgaccggatcttcc-gcagcaacctg-gtt | 459 |
| AVIA_ORF37: | 460 | tcccgcggccgggcgctgctgctcctgctgctgttctccgacaccggcgag-gaggacgc | 518 |
| | | \|\| \|\| \|\|\|\|\|\|\|\| \|\|\|\|\|\| \| \|\|\|\|\| \|\| \| \|\|\|\|\|\|\|\|\|\|\|\|\|\|\|\| \| \|\|\|\|\| | |
| EVER_ORF12: | 460 | tcgcgtggccgggccctgctggtgctgctcctctactccgacaccggcgagcg-tgacgc | 518 |
| AVIA_ORF37: | 519 | gcccacgctgatccgcgtcggctcccacctggacgtaccgccgctgctggcaccgtacgg | 578 |
| | | \|\|\|\|\|\|\|\|\|\|\|\|\|\|\| \|\|\|\|\| \|\| \|\|\|\|\|\|\|\|\|\|\| \|\|\|\|\| \|\|\|\|\|\|\|\| \|\| \|\|\|\|\| | |
| EVER_ORF12: | 519 | gcccacgctgatccgggtcggttcgcacctggacgtgccgcccctgctggcgccctacgg | 578 |
| AVIA_ORF37: | 579 | cgccgagggcacctacctggaggcc-g--gggaggtgggacg-ggaccggccgct---ga | 631 |
| | | \|\|\|\|\|\|\|\|\| \|\|\|\|\|\|\|\| \|\|\| \| \| \|\| \|\|\|\|\| \|\| \|\|\|\|\|\| \|\| \|\| \|\| | |
| EVER_ORF12: | 579 | cgccgaggggacctacct---cgcctgccgcgacgtggg-cgcggaccgccccctcgcga | 634 |
| | | HOXG-AS1 | |
| | | <<<<<<<<<<<<<<<<<<<<<<<<<<< | |
| AVIA_ORF37: | 632 | -ggtccgcga-cgggcaaggccggg-gacgcctacctctgccaccccttcctggtgcaca | 688 |
| | | \|\| \|\| \| \|\|\|\|\| \|\| \|\|\|\| \|\|\|\|\|\|\|\|\|\|\|\|\|\|\|\|\| \|\| \|\|\|\|\|\|\|\|\|\|\|\|\| | |
| EVER_ORF12: | 635 | tgg-cc---accgggc--gggcgggcgacgcctacctctgccatccgttcctggtgcaca | 688 |
| AVIA_ORF37: | 689 | cgccggtcgccaacaccggcgtcc-gcccgcgcttcatggcccagccgaacct-gctgc- | 745 |
| | | \|\|\|\|\| \|\| \|\|\|\|\|\|\|\|\|\|\| \|\| \|\|\|\| \|\| \|\|\|\|\|\|\|\|\|\|\|\|\| \|\|\| \|\|\|\|\| | |
| EVER_ORF12: | 689 | cgccgatcaccaacaccggc-accagcccccggttcatggcccagc----cctcgctgca | 743 |
| AVIA_ORF37: | 746 | -ccgtggggc-agctcgaactcgaccggc-ccgacggccggtacacccccgtcgagcggg | 802 |
| | | \|\|\| \|\|\| \|\| \|\|\|\| \|\| \|\|\|\| \|\| \|\|\|\|\|\|\| \| \|\|\|\| \|\|\| \|\|\|\|\|\|\|\|\|\| | |
| EVER_ORF12: | 744 | accgaccggcgagttcgacctggacc-gcgccgacgggcagtacgtcccggtcgagcggg | 802 |
| AVIA_ORF37: | 803 | ccg-tgcgccggg 814 | |
| | | \|\| \| \|\|\|\|\| | |
| EVER_ORF12: | 803 | -cgat---ccggg 811 | |

Identities = 653/853 (76%),
Gaps = 99/853 (11%)

The oligonucleotide sequences listed below on Table XXVI were supplied by Invitrogen™. Where necessary, degenerate oligonucletides were designed in which “S” denotes a base in the oligonucleotide that consists of an approximately equimolar mixture of G and C, and in which “R” denotes a base in the oligonucleotide that consists of an approximately equimolar mixture of G and A. The oligonucleotides may be used as hybridization probes to identify orthosomycin genes as further described in Example 7. The oligonucleotides may also be used as PCR primers, as described in Example 8, to amplify portions of orthosomycin genes either from isolated DNA (from pure cultures, mixed cultures, or environmental samples) or directly from crude cell mass or environmental sample. As further members of each gene family disclosed in this application are identified, those skilled in the art will be able to improve and refine diagnostic oligonucleotides for identifying and isolating orthosomycin genes, for example by using appropriate tools capable of carrying out multiple sequence alignments, for example Clustal (Higgins.and Sharp (1988) Gene Vol. 73 pp.237-244).

TABLE XXVI

| | | |
|---|---|---|
| Oligo- | | |
| nucleotide | Sequence (5′->3′) | length (nt) |

| | | |
|---|---|---|
| UEVA-S1 | ATCGACGGCGTCTACGGCCGGTGCTGC | 27 |
| UEVA-S2 | TCSCGSTACGCCAAGGACAACCCGGAC | 27 |
| UEVA-AS1 | GTCGATSACCGCGGACGACCACGACGG | 27 |
| UEVA-AS2 | GAASACCTCGCCGACGCCGTCGCAGGA | 27 |
| UEVB-S1 | CTGCACTACCACCCGCACTTCGT | 23 |
| UEVB-CTL1* | TCCCACACGCCGTCGCCGASSTGG | 24 |
| HOXG-S1 | ACTACGGCTGGCACATCGACGCCAGCT | 27 |
| HOXG-AS1 | CAGGAASGGRTGGCAGAGGTAGGCGTC | 27 |

*This oligonucleotide serves as a negative control in the hybridization experiments.

### EXAMPLE 7

### Use of Diagnostic Nucleic Acid Sequences for Identifying Orthosomycin Genes by Hybridization:

The microorganism Micromonospora carbonacea var. africana NRRL 15099 was obtained from the Agriculture Research Service Culture Collection of the United States Department of Agriculture. This organism was propagated on N-Z amine agar medium (per liter of water: 10.0 g glucose, 20 g soluble starch, 5.0 g yeast extract, 5.0 g N-Z Amine Type A (Sigma C0626), 1.0 g reagent grade CaCO₃, 15.0 g agar) at 28 degrees Celsius for several days. For isolation of high molecular weight genomic DNA, cell mass from three freshly grown, near confluent 100 mm petri dishes was used. The cell mass was collected by gentle scraping with a plastic spatula. Residual agar medium was removed by repeated washes with STE buffer (75 mM NaCl; 20 mM Tris-HCl, pH 8.0; 25 mM EDTA). High molecular weight DNA was isolated by established protocols (Kieser et al., Practical Streptomyces Genetics, The John Innes Foundation, 2000) and its integrity was verified by field inversion gel electrophoresis (FIGE) using the preset program number 6 of the FIGE MAPPER™ power supply (BIORAD).

A Micromonospora carbonacea var. africana genomic DNA cosmid library was prepared using the SuperCos-1 cosmid vector (Stratagene™). The cosmid arms were prepared as specified by the manufacturer. The high molecular weight DNA was subjected to partial digestion at 37 degrees Celsius with approximately one unit of Sau3AI restriction enzyme (New England Biolabs) per 100 micrograms of DNA in the buffer supplied by the manufacturer. At various timepoints, aliquots of the digestion were transferred to new microfuge tubes and the enzyme was inactivated by adding a final concentration of 10 mM EDTA and 0.1% SDS. Aliquots judged by FIGE analysis to contain a significant fraction of DNA in the desired size range (30-50 kb) were pooled, extracted with phenol/chloroform (1:1 vol:vol), and pelletted by ethanol precipitation. The 5′ ends of Sau3AI DNA fragments were dephosphorylated using alkaline phosphatase (Roche) according to the manufacturer's specifications at 37 degrees Celcius for 30 min. The phosphatase was heat inactivated at 70 degrees Celcius for 10 min and the DNA was extracted with phenol/chloroform (1:1 vol:vol), pelletted by ethanol precipitation, and resuspended in sterile water. The dephosphorylated Sau3AI DNA fragments were then ligated overnight at room temperature to the SuperCos-1 cosmid arms in a reaction containing approximately four-fold molar excess SuperCos-1 cosmid arms. The ligation products were packaged using Gigapack® III XL packaging extracts (Stratagene™) according to the manufacturer's specifications. A library of 864 isolated cosmid clones was picked and inoculated into nine 96-well microtiter plates containing LB broth (per liter of water: 10.0 g NaCl; 10.0 g tryptone; 5.0 g yeast extract) which were grown overnight and then adjusted to contain a final concentration of 25% glycerol. These microtiter plates were stored at −80 degrees Celcius and served as glycerol stocks. Duplicate microtiter plates were arrayed onto nylon membranes as follows. Cultures grown on microtiter plates were concentrated by pelleting and resuspending in a small volume of LB broth. A 3×3 grid of 96-pins per grid was spotted onto nylon membranes. These membranes representing the complete cosmid library were then layered onto LB agar and incubated ovenight at 37 degrees Celcius to allow colonies to grow. The membranes were layered onto filter paper pre-soaked with 0.5 N NaOH/1.5 M NaCl for 10 min to denature the DNA and then neutralized by transferring onto filter paper pre-soaked with 0.5 M Tris (pH 8)/1.5 M NaCl for 10 min. Cell debris was gently scraped off with a plastic spatula and the DNA was crosslinked onto the membranes by UV irradiation using a GS GENE LINKER™ UV Chamber (BIORAD).

Orthosomycin-specific hybridization oligonucleotide probes were radiolabeled with P³² using T4 polynucleotide kinase (New England Biolabs) in 15 microliter reactions containing 5 picomoles of oligonucleotide and 6.6 picomoles of [γ-P³²]ATP in the kinase reaction buffer supplied by the manufacturer. After 1 hour at 37 degrees Celcius, the kinase reaction was terminated by the addition of EDTA to a final concentration of 5 mM. The specific activity of the radiolabeled oligonucleotide probes was estimated using a Model 3 Geiger counter (Ludlum Measurements Inc., Sweetwater, Tex.) with a built-in integrator feature. The radiolabeled oligonucleotide probes were heat-denatured by incubation at 85 degrees Celcius for 10 minutes and quick-cooled in an ice bath immediately prior to use.

Cosmid library membranes were prepared by incubation for at least 2 hours at 42 degrees Celcius in Prehyb Solution (6× SSC; 20 mM NaH₂PO₄; 5× Denhardt's; 0.4% SDS; 0.1 mg/ml sonicated, denatured salmon sperm DNA) using a hybridization oven with gentle rotation. The membranes were then placed in Hyb Solution (6× SSC; 20mM NaH₂PO₄; 0.4% SDS; 0.1 mg/ml sonicated, denatured salmon sperm DNA) containing 1×10⁶ cpm/ml of radiolabeled oligonucleotide probe and incubated overnight at 42 degrees Celcius using a hybridization oven with gentle rotation. The next day, the membranes were washed with Wash Buffer (6× SSC, 0.1% SDS) for 45 minutes each at 46, 48, and 50 degrees Celcius using a hybridization oven with gentle rotation. The membranes were then exposed to X-ray film to visualize and identify the positive cosmid clones. The results obtained with four representative orthosomycin-specific oligonucleotide probes are shown in Table XXVII. Cosmid clones that were positive in the hybridization experiment are indicated by a ‘+’. The ends of the inserts in these cosmids were sequenced using T7 and T3 universal primers and, as expected, were shown to contain sequences homologous to those in the EVER locus (data not shown). Since cosmid clone IHO1was detected by most of the orthosomycin-specific oligonucleotide probes, including one derived from the OXCO gene family from the EVER locus (data not shown), it was selected for further sequencing analysis. This cosmid clone was completely sequenced using a shotgun method. Cosmid clones FBO3 and DHO1 were found to overlap and extend the IHOl sequence towards the 5′ and 3′ direction, respectively, so they too were sequenced. Together, overlapping cosmid clones IH01, FB03, and DH01 (hereto referred to as 050CB, 050CA, and 050CG, respectively) constitute over 85 kilobasepairs that includes the everninomicin biosynthetic locus of Micromonospora carbonacea var. africana (EVEA). EVEA is further described in Example 10.

TABLE XXVII

| | oligonucleotide probe | | | |
|---|---|---|---|---|
| Cosmid clone | UEVA-S2 | UEVA-AS2 | HOXG-S1 | HOXG-AS1 |
| AF02 | | | + | + |
| CD01 | + | | | |
| DA01 | + | | | |
| DD06 | | | | + |
| DH01 | | | + | |
| FB03 | | | | + |
| FH08 | + | | | |
| FH09 | | + | | |
| GF08 | | + | | |
| HA08 | | + | | |
| HD01 | | + | | |
| HF10 | | | | + |
| HH12 | | | + | |
| IB04 | | + | | |
| ID08 | | | + | + |
| IF12 | | + | | |
| IH01 | + | + | | + |

To verify the specificity of the diagnostic probes according to the invention, 50 ng aliquots of cosmid DNA from three microorganisms known to contain orthosomycin biosynthetic loci were spotted onto nylon membranes and denatured, crosslinked and probed as described above. Cosmid DNA was isolated according to the alkaline lysis method (Sambrook et al. 1989 Molecular cloning: a laboratory manual, 2^{nd} edition. Cold Spring Harbour Laboratory, Cold Spring Harbour, N.Y.) from 15 mililiter cultures. Cosmids used in this experiment included 050CA, 050CB, and 050CG of the everninomicin locus from Micromonospora carbonacea var. africana (EVEA); 010CA, 010CB, and 010CG of the everninomicin locus from Micromonospora carbonacea var. aurantiaca (EVER); and 017CH, 017CP, and 017CP of the avilamycin-type locus from Streptomyces mobarensis (AVIA). In addition, a Micromonospora carbonacea var. aurantiaca genomic DNA cosmid clone, 050CC, which is unrelated to orothosomycin loci served as a negative control. The results obtained with eight orthosomycin-specific oligonucleotide probes are shown in Table XXVIII. Cosmid clones that were positive in the hybridization experiment are indicated by a ‘+’. Cosmid clones that were negative in the hybridization experiment are indicated by a ‘−’.

The results of the experiment summarized in Table XXVIII are consistent with the sequence information available for EVER, EVEA, and AVIA. The members of the UEVA, HOXG, and UEVB protein families in EVER are all contained within the 010CA cosmid; the same is not true for the other two loci, i.e. the members of the UEVA, HOXG and UEVB protein families in EVEA and AVIA are more distant to one another. All four UEVA probes consistently detected the same cosmid(s) in EVER, EVEA and AVIA, although the UEVA-S2 probe gave a weak signal for EVEA (indicated by the parentheses in Table XXVIII). The UEVB-S1 probe did not hybridize to EVEA cosmids as EVEA does not contain a UEVB homologue (see Example 10). None of the oligonucleotide probes hybridized to the negative control cosmid DNA, 050CC. The negative control oligonucleotide probe UEVB-CTL1 did not hybridize with any of the cosmid DNAs.

TABLE XXVIII

| | Cosmid | Oligonucleotide probe | | | | | | | |
|---|---|---|---|---|---|---|---|---|---|
| LOCUS | clone | UEVA-S1 | UEVA-S2 | UEVA-AS1 | UEVA-AS2 | HOXG-S1 | HOXG-AS1 | UEVB-S1 | UEVB-CTL1 |
| EVER | 010CA | + | + | + | + | + | + | + | − |
| | 010CB | − | − | − | − | − | − | − | − |
| | 010CG | − | − | − | − | − | − | − | − |
| EVEA | 050CA | − | − | − | − | − | − | − | − |
| | 050CB | + | (+) | + | + | − | − | − | − |
| | 050CG | − | − | − | − | + | + | − | − |
| AVIA | 017CH | + | + | + | + | − | − | + | − |
| | 017CP | + | + | + | + | + | + | | − |
| | 017CR | − | − | − | − | − | − | + | − |
| control | 050CC | − | − | − | − | − | − | − | − |

### EXAMPLE 8

### Use of Diagnostic Nucleic Acid Sequences for Identifying Orthosomycin Genes by PCR Amplification

The oligonucleotides described in Example 6 may be used as PCR primers to identify orthosomycin genes and biosynthetic loci and/or orthosomycin-producing organisms. Genomic DNA was prepared from Micromonospora carbonacea var. africana and Micromonospora carbonacea var. aurantiaca as described in Example 7. 010CA cosmid DNA was prepared by the alkaline lysis method (Sambrook et al. 1989 Molecular cloning: a laboratory manual, 2^{nd} edition. Cold Spring Harbour Laboratory, Cold Spring Harbour, NY). Aliquots of the genomic DNA and the cosmid DNA were used as template DNA in PCR reactions with the following four PCR primer pairs: 1) UEVA-S2 and UEVA-AS1; 2) UEVA-S1 and UEVA-AS1; 3) UEVA-S2 and UEVA-AS2; and 4) UEVA-S1 and UEVA-AS2.

Each PCR amplification was carried out in 50 microliter reactions containing 50-100 nanograms of template DNA; 37.5 picomoles of each primer; a final concentration of 0.2 mM each of dATP, dGTP, dCTP, and dTTP; a final concentration of 10% dimethyl sulfoxide, and 2 units of Pfu DNA polymerase (Stratagene™) in the reaction buffer supplied with the enzyme by the manufacturer. The PCR conditions included an initial two minute denaturation step at 96 degrees Celcius followed by thirty amplification cycles in which denaturation was performed at 96 degrees Celcius for 30 seconds, annealing was performed at 45 degrees Celcius for 30 seconds, and extension was performed at 72 degrees Celcius for 2.5 minutes.

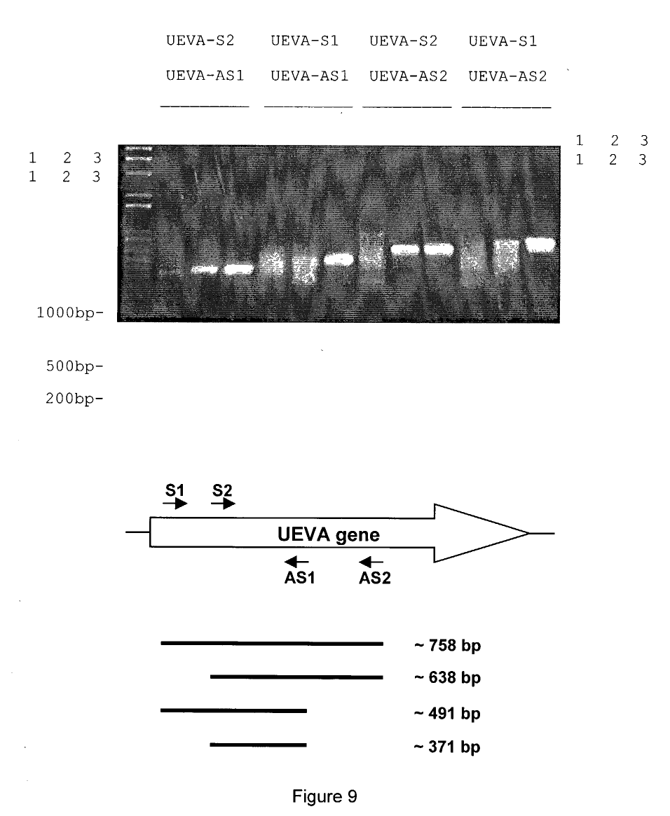

The four primer pairs used were expected to amplify portions of the orthosomycin-specific UEVA gene and are listed in the order of increasing expected size for the amplified product. The relative position of these oligonucleotides is depicted on the UEVA aligned nucleotide sequences as shown below and in FIG. 9.

FIG. 9 is a picture of a 1% agarose gel stained with ethidium bromide in which 5 microliter aliquots of the PCR reactions were resolved by electrophoresis. Primer pairs are indicated at the top of the Figure. The numbers indicate which template DNA was used in the PCR reaction, i.e. “1” represents Micromonospora carbonacea var. africana genomic DNA; “2” represents Micromonospora carbonacea var. aurantiaca genomic DNA; and “3” represents cosmid 010CA from the EVER locus. The leftmost lane contains the 1 Kb Plus DNA ladder (Invitrogen™) molecular weight standards, some of which are labeled to the left in basepairs (bp). The schematic drawing below the picture in FIG. 9 depicts the relative positions of the primer pairs and the expected sizes (in basepairs) of the PCR products based on the known nucleotide sequence of the UEVA gene from the EVER locus (described in Example 1).

Referring to FIG. 9, the PCR reactions in which genomic DNA was used as template produced a smear with all four primer pairs tested. In contrast, the-PCR reactions in which purified 010CA cosmid DNA was used as template gave rise to distinct bands that are consistent with the expected sizes. This result suggests that the PCR conditions used are suboptimal for amplification from genomic DNA but may be adequate for less complex, subcloned DNA fragments. The smears that arise with genomic DNA templates are likely due to mispriming (i.e., inaccurate annealing of the PCR primers followed by extension) caused by a combination of a suboptimal annealing temperature in the thermal cycle, a high G/C content and complexity of the genomic DNA, relatively low abundance of the target sequence, and the presence of some degenerate positions in the oligonucleotide PCR primers.

Based on the assumption that a certain proportion of the amplified products arise from accurate priming events (as can be seen in several lanes in FIG. 9), an aliquot of the products obtained with the UEVA-S1 and UEVA-AS2 primer pair was used as template DNA in a second PCR reaction using the UEVA-S2 and UEVA-AS1 primer pair so as to specifically amplify the UEVA sequences. In essence, this amounts to a two-step nested PCR in which the first round of amplification serves to enrich for UEVA sequences with a pair of “outer” UEVA-derived primers and the second round of amplification, carried out with primers that are contained within the region defined by the “outer” primers. Using this two-step nested PCR approach on both Micromonospora carbonacea var. africana genomic DNA and Micromonospora carbonacea var. aurantiaca genomic DNA, a distinct band was obtained whose size was similar to that obtained with cosmid 010CA using the UEVA-S2 and UEVA-AS1 primer pair (data not shown). The band was resolved on an agarose gel and purified by spinning through a glass wool plug, extraction with phenol/chloroform (1:1 vol:vol), and pelletting by ethanol precipitation. The purified DNA was then sequenced using the UEVA-S2 and UEVA-AS1 primers.

The sequencing of the M. carbonacea var. africana PCR product yielded 302 nucleotides of high quality sequence information which is in perfect agreement with the region coding for amino acids 69-168 of the UEVA protein in EVEA (described in Example 10):

| | |
|---|---|
| AACCCCGGCCGGGTGATGGGCCTGGCGGACGCCTTCAACAGCCCC | 45 |
| N P G R V M G L A D A F N S P | |
| AACATGCGCCGGACCCGGCTGGCGATGCTGGCCGGGGAGCGGGTC | 90 |
| N M R R T R L A M L A G E R V | |
| GACGCCTGCTCCTACTGCTACCACCGCGAGGACCACGGCGCGCTG | 135 |
| D A C S Y C Y H R E D H G A L | |
| TCGTACCGGCAGGAGATCAACCAGCGGTTCCGGGACATCGCCGAC | 180 |
| S Y R Q E I N Q R F R D I A D | |
| CCCGACCGGCTGGCCGCCCGCACCGCGCCCGACGGCACCGTCGAG | 225 |
| P D R L A A R T A P D G T V E | |
| GACTTCCCGTTCTTCCTCGACATCCGGTTCGGCAACACCTGCAAC | 270 |
| D F P F F L D I R F G N T C N | |
| CTGCGGTGCGTGATGTGCGCGTACCCGGTCAG 302 | |
| L R C V M C A Y P V | |

The sequencing of the M. carbonacea var. aurantiaca PCR product yielded 343 nucleotides of high quality sequence information which is in perfect agreement with the region coding for amino acids 72-185 of the UEVA protein in the EVER locus (described in Example 1):

| | |
|---|---|
| CGGTACGCCAAGGACAACCCGGACCGCGTGATGGGCATCCGGGAG | 45 |
| R Y A K D N P D R V M G I R E | |
| GCCTTCAACAGCCCCAACATGAAGCGGACCCGGCTGGCGATGCTC | 90 |
| A F N S P N M K R T R L A M L | |
| GGTGGCGAGCGCGTGGAGGCGTGCAAGTACTGCTACTTCCGGGAG | 135 |
| C G E R V E A C K Y C Y F R E | |
| GACCACGGCGCCCAGTCCTACCGGCAGAACGTCAACCGCCGGTTC | 180 |
| D H C A Q S Y R Q N V N R R E | |
| CACCAGGAGTACGACCTCGATGCGCTCGCCGCCCGTACCGCCGCG | 225 |
| H Q E Y D L D A L A A R T A A | |
| GACGGCACGGTCGAGGAGTTCCCGTTCTTTCTCGACATCAGGTTC | 270 |
| D C T V E E F P F F L D I R F | |
| GGCAACCTCTGCAACCTGCGGTGCGTCATGTGCACCTACCCGGTG | 315 |
| G N L C N L R C V M C T Y P V | |
| AGTTCCTCCTGGGGCGCCAAGCAACGCC 343 | |
| S S S W G A K Q R | |

### EXAMPLE 9

### In silico Identification of Orthosomycin Biosynthetic Genes

Sequence information from the polypeptides and polynucleotides taught in the invention allows for in silico identification of orthosomycin biosynthetic loci in any biological sample. The biological sample may be an environmental sample (i.e. soil), genetic material and purified genetic material (DNA, RNA, cDNA) from environmental samples or from cultivated microorganisms. Genomic DNA from cultured Micromonospora carbonacea var. africana NRRL 15009 was extracted and analyzed as described in Canadian patent application 2,352,451. Briefly, extracted genomic DNA was randomly fragmented, size-fractionated to generate small size DNA fragments and cloned into an appropriate plasmid vector to generate a Genomic Sampling Library (GSL). The GSL is a library of small size random genomic DNA fragments that covers the entire genome of Micromonospora carbonacea var. africana NRRL 15009.

The GSL library was analyzed by sequence determination of the cloned genomic DNA inserts. The universal primers KS and/or SK, referred to as forward (F) and reverse (R) primers respectively, were used to initiate polymerization of labeled DNA. Sequence analysis of the Genomic Sequence Tags (GSTs) generated was performed using a 3700 ABI capillary electrophoresis DNA sequencer (Applied Biosystems). Further analysis of the GSTs was performed by sequence homology comparison to various protein sequence databases. The DNA sequences of the obtained GSTs were translated into amino acid sequences and compared to the National Center for Biotechnology Information (NCBI) nonredundant protein database and the DECIPHER™ database (Ecopia BioSciences, St.-Laurent, QC, Canada) using previously described algorithms (Altschul et al. J. Mol. Biol., October 5; 215(3) 403-10). Sequence similarity with known proteins of defined function in the databases facilitates recognition of protein families of the invention from the polypeptides encoded by the translated GSTs.

Four hundred GSTs were analyzed from the Micromonospora carbonacea var. africana GSL library and compared to the above protein databases. Among the 400 analyzed GSTs, three GSTs (RAA12, RAC92, FAE38) were found to have substantial sequence similarity to proteins taught by the invention to be diagnostic of orthosomycin biosynthetic loci (HOXG, OXRW, MTFD, respectively). These three GSTs had a much greater degree of similarity to homologous proteins from orthosomycin-specifying loci than to related proteins from non-orthosomycin-encoding loci. The degree of homology between the translated GST products and their homologs in EVER, AVIA, and AVIL othosomycin loci is shown in Table XXIX. All three GSTs encode members of protein families that are unique to the biosynthesis of orthosomycin compounds. HOXG, OXRW, and MTFD are only found in orthosomycin-encoding loci and their detection through the genomic sampling of Micromonospora carbonacea var. africana clearly indicates the presence of an orthosomycin-specific locus within the genome of the microorganism. The GSTs used for the in silico determination of the orthosomycin locus were subsequently shown to belong to EVEA as confirmed by complete sequence determination of the EVEA locus (see example 10).

Further determination of the class of the predicted orthosomycin compound would have been possible if GSTs harboring members of the protein families diagnostic for everninomicins or avilamycins had been detected. The presence of the orthosomycin-specifying locus was confirmed by detection and complete sequence determination of the locus (see example 10

A similar approach was used to evaluate the potential of Streptomyces sp. (collection ATCC 39365) to encode orthosomycin compounds. Seven hundred GSTs were analyzed and compared to protein databases. Among these GSTs, two (FAF63, FAA47) were shown to have substantial sequence homology to HOXG and PKSO protein families that are found in orthosomycin loci (see Table XXIX). HOXG is an orthosomycin diagnostic protein family as it is only found in orthosomycin biosynthetic loci, whereas PKSO is a protein family found in orthosomycin loci, but may also be associated with secondary metabolism other than orthosomycin biosynthesis. Use of the compositions and methods of the invention in regard to Streptomyces sp. (collection ATCC 39365) demonstrates the predictive ability of the invention for discovery of orthosomycin loci in microorganisms or biomass for which no metabolite expression determination was previously performed.

Table XXIX presents comparison of translated GSTs from Micromonospora carbonacea var. africana and Streptomyces sp. with their homologs from orthosomycin loci. Blast analysis was performed using the Blastx algorithm (Altschul et al. J. Mol. Biol., October 5; 215(3) 403-10). In each comparison, the first line indicates the number of identical amino acids and the degree of identity whereas the second line indicates the number of similar amino acids and the degree of similarity between the two protein segments.

TABLE XXIX

| | | | |
|---|---|---|---|
| EVER | EVEA | AVIA | AVIL |

Micromonospora carbonacea var. africana

| | | | | |
|---|---|---|---|---|
| HOXG | 75/93 (80%) | 86/86 (100%) iden. | 78/94 (82%) | 78/93 (83%) |
| | iden. | | | |
| (RAA12) | 77/93 (82%) | NA | 85/94 (89%) | 86/93 (91%) |
| | sim. | | | |
| OXRW | 83/132 (62%) | 134/134 (100%) | 86/132 (65%) | 87/132 (65%) |
| (RAC92) | 102/132 (76%) | NA | 101/132 (76%) | 102/132 (76%) |
| MTFD | 56/87 (64%) | 105/105 (100%) | 50/91 (54%) | 48/91 (52%) |
| (FAE38) | 70/87 (80%) | NA | 63/91 (68%) | 62/91 (67%) |

Streptomyces sp.

| | | | | |
|---|---|---|---|---|
| HOXG | 47/99 (47%) | 47/99 (47%) | 52/109 (47%) | 49/109 (44%) |
| (FAF63) | 56/99 (56%) | 56/99 (56%) | 63/109 (57%) | 61/109 (55%) |
| PKSO | 81/146 (55%) | 76/146 (52%) | 83/146 (56%) | 82/146 (56%) |
| (FAA47) | 97/146 (65%) | 92/146 (62%) | 97/146 (65%) | 95/146 (64%) |

### EXAMPLE 10

### The Everninomicin Biosynthetic Locus in Micromonospora carbonacea var. africana

The microorganism Micromonospora carbonacea var. africana NRRL 15099 was obtained from the Agriculture Research Service Culture Collection of the United States Department of Agriculture, 1815 N. University Street, Peoria, Ill. 61604. The everninomicin compounds produced by strain NRRL 15099 are described in U.S. Pat. No. 4,597,968. The biosynthetic locus for everninomicin from strain NRRL 15099 (EVEA) was identified according to the method described in Canadian patent application CA 2,352,451. The sequences obtained from cosmids containing overlapping genomic inserts spanning EVEA were identified. Within the sequences of the cosmid inserts, numerous ORFs encoding polypeptides having homology to known proteins were identified. Contiguous nucleotide sequences and deduced amino acid sequences of EVEA are provided as follows: the amino acid sequence of ORF 1 (SEQ ID NO 271) is deduced from the nucleic acid sequence of SEQ ID NO 272 drawn from contig 1 (SEQ ID NO 278). The amino acid sequence of ORF 2 (SEQ ID NO 137) is deduced from the nucleic acid sequence of SEQ ID NO 138 drawn from contig 1 (SEQ ID NO 278). The amino acid sequence of ORF 3 (SEQ ID NO 5) is deduced from the nucleic acid sequence of SEQ ID NO 6 drawn from contig 1 (SEQ ID NO 278). The amino acid sequence of ORF 4 (SEQ ID NO 37) is deduced from the nucleic acid sequence of SEQ ID NO 38 drawn from contig 1 (SEQ ID NO 278). The amino acid sequence of ORF 5 (SEQ ID NO 171) is deduced from the nucleic acid sequence of SEQ ID NO 172 drawn from contig 1 (SEQ ID NO 278). The amino acid sequence of ORF 6 (SEQ ID NO 173) is deduced from the nucleic acid sequence of SEQ ID NO 174 drawn from contig 1 (SEQ ID NO 278). The amino acid sequence of ORF 7 (SEQ ID NO 49) is deduced from the nucleic acid sequence of SEQ ID NO 50 drawn from contig 1 (SEQ ID NO 278). The amino acid sequence of ORF 8 (SEQ ID NO 103) is deduced from the nucleic acid sequence of SEQ ID NO 104 drawn from contig 1 (SEQ ID NO 278). The amino acid sequence of ORF 9 (SEQ ID NO 269) is deduced from the nucleic acid sequence of SEQ ID NO 270 drawn from contig 1 (SEQ ID NO 278). The amino acid sequence of ORF 10 (SEQ ID NO 109) is deduced from the nucleic acid sequence of SEQ ID NO 110 drawn from contig 1 (SEQ ID NO 278). The amino acid sequence of ORF 11 (SEQ ID NO 157) is deduced from the nucleic acid sequence of SEQ ID NO 158 drawn from contig 1 (SEQ ID NO 278). The amino acid sequence of ORF 12 (SEQ ID NO 115) is deduced from the nucleic acid sequence of SEQ ID NO 116 drawn from contig 1 (SEQ ID NO 278). The amino acid sequence of ORF 13 (SEQ ID NO 121) is deduced from the nucleic acid sequence of SEQ ID NO 122 drawn from contig 1 (SEQ ID NO 278). The amino acid sequence of ORF 14 (SEQ ID NO 197) is deduced from the nucleic acid sequence of SEQ ID NO 198 drawn from contig 1 (SEQ ID NO 278). The amino acid sequence of ORF 15 (SEQ ID NO 91) is deduced from the nucleic acid sequence of SEQ ID NO 92 drawn from contig 1 (SEQ ID NO 278). The amino acid sequence of ORF 16 (SEQ ID NO 185) is deduced from the nucleic acid sequence of SEQ ID NO 186 drawn from contig 2 (SEQ ID NO 279). The amino acid sequence of ORF 17 (SEQ ID NO 85) is deduced from the nucleic acid sequence of SEQ ID NO 86 drawn from contig 2 (SEQ ID NO 279). The amino acid sequence of ORF 18 (SEQ ID NO 227) is deduced from the nucleic acid sequence of SEQ ID NO 228 drawn from contig 2 (SEQ ID NO 279). The amino acid sequence of ORF 19 (SEQ ID NO 239) is deduced from the nucleic acid sequence of SEQ ID NO 240 drawn from contig 2 (SEQ ID NO 279). The amino acid sequence of ORF 20 (SEQ ID NO 79) is deduced from the nucleic acid sequence of SEQ ID NO 80 drawn from contig 2 (SEQ ID NO 279). The amino acid sequence of ORF 21 (SEQ ID NO 275) is deduced from the nucleic acid sequence of SEQ ID NO 276 drawn from contig 2 (SEQ ID NO 279). The amino acid sequence of ORF 22 (SEQ ID NO 11) is deduced from the nucleic acid sequence of SEQ ID NO 12 drawn from contig 2 (SEQ ID NO 279). The amino acid sequence of ORF 23 (SEQ ID NO 43) is deduced from the nucleic acid sequence of SEQ ID NO 44 drawn from contig 2 (SEQ ID NO 279). The amino acid sequence of ORF 24 (SEQ ID NO 143) is deduced from the nucleic acid sequence of SEQ ID NO 144 drawn from contig 2 (SEQ ID NO 279). The amino acid sequence of ORF 25 (SEQ ID NO 17) is deduced from the nucleic acid sequence of SEQ ID NO 18 drawn from contig 2 (SEQ ID NO 279). The amino acid sequence of ORF 26 (SEQ ID NO 191) is deduced from the nucleic acid sequence of SEQ ID NO 192 drawn from contig 2 (SEQ ID NO 279). The amino acid sequence of ORF 27 (SEQ ID NO 61) is deduced from the nucleic acid sequence of SEQ ID NO 62 drawn from contig 2 (SEQ ID NO 279). The amino acid sequence of ORF 28 (SEQ ID NO 31) is deduced from the nucleic acid sequence of SEQ ID NO 32 drawn from contig 2 (SEQ ID NO 279). The amino acid sequence of ORF 29 (SEQ ID NO 179) is deduced from the nucleic acid sequence of SEQ ID NO 180 drawn from contig 2 (SEQ ID NO 279). The amino acid sequence of ORF 30 (SEQ ID NO 163) is deduced from the nucleic acid sequence of SEQ ID NO 164 drawn from contig 2 (SEQ ID NO 279). The amino acid sequence of ORF 31 (SEQ ID NO 67) is deduced from the nucleic acid sequence of SEQ ID NO 68 drawn from contig 2 (SEQ ID NO 279). The amino acid sequence of ORF 32 (SEQ ID NO 207) is deduced from the nucleic acid sequence of SEQ ID NO 208 drawn from contig 2 (SEQ ID NO 279). The amino acid sequence of ORF 33 (SEQ ID NO 55) is deduced from the nucleic acid sequence of SEQ ID NO 56 drawn from contig 2 (SEQ ID NO 279). The amino acid sequence of ORF 34 (SEQ ID NO 25) is deduced from the nucleic acid sequence of SEQ ID NO 26 drawn from contig 2 (SEQ ID NO 279). The amino acid sequence of ORF 35 (SEQ ID NO 223) is deduced from the nucleic acid sequence of SEQ ID NO 224 drawn from contig 2 (SEQ ID NO 279). The amino acid sequence of ORF 36 (SEQ ID NO 235) is deduced from the nucleic acid sequence of SEQ ID NO 236 drawn from contig 2 (SEQ ID NO 279). The amino acid sequence of ORF 37 (SEQ ID NO 211) is deduced from the nucleic acid sequence of SEQ ID NO 212 drawn from contig 2 (SEQ ID NO 279). The amino acid sequence of ORF 38 (SEQ ID NO 231) is deduced from the nucleic acid sequence of SEQ ID NO 232 drawn from contig 2 (SEQ ID NO 279). The amino acid sequence of ORF 39 (SEQ ID NO 219) is deduced from the nucleic acid sequence of SEQ ID NO 220 drawn from contig 2 (SEQ ID NO 279). The amino acid sequence of ORF 40 (SEQ ID NO 215) is deduced from the nucleic acid sequence of SEQ ID NO 216 drawn from contig 2 (SEQ ID NO 279). The amino acid sequence of ORF 41 (SEQ ID NO 243) is deduced from the nucleic acid sequence of SEQ ID NO 244 drawn from contig 2 (SEQ ID NO 279). The amino acid sequence of ORF 42 (SEQ ID NO 273) is deduced from the nucleic acid sequence of SEQ ID NO 274 drawn from contig 2 (SEQ ID NO 279). The amino acid sequence of ORF 43 (SEQ ID NO 73) is deduced from the nucleic acid sequence of SEQ ID NO 74 drawn from contig 2 (SEQ ID NO 279). The amino acid sequence of ORF 44 (SEQ ID NO 97) is deduced from the nucleic acid sequence of SEQ ID NO 98 drawn from contig 2 (SEQ ID NO 279). The amino acid sequence of ORF 45 (SEQ ID NO 131) is deduced from the nucleic acid sequence of SEQ ID NO 132 drawn from contig 2 (SEQ ID NO 279). Homology was determined using the BLASTP version 2.2.2 algorithm with the default parameters. Table XXX-A presents the results of the homology analysis. Table XXX-B presents the position, length and orientation of each EVEA ORF within SEQ ID NOS: 278 and 279.

TABLE XXX-A
EVEA blast table

| ORF | Family | #aa | GenBank homology | probability | | | |
|---|---|---|---|---|---|---|---|
| 1 | HYDH | 248 | CAB95990 1, 229aa | 1e−43 | 100/201 (49.75%) | 115/201 (57.21%) | putative hydrolase, Streptomyces |
| | | | | | | | coelicolor |
| 2 | NUTA | 355 | A26984, 355 aa | 1e−127 | 218/353 (61.76%) | 272/353 (77.05%) | strD protein, Streptomyces |
| | | | | | | | griseus |
| | | | P08075, 355aa | 1e−126 | 217/353 (61.47%) | 272/353 (77.05%) | dTDP-glucose synthase, Streptomyces |
| | | | | | | | griseus |
| | | | T30872, 355aa | 1e−123 | 213/354 (60.17%) | 273/354 (77.12%) | dNDP-glucose synthase, Streptomyces |
| | | | | | | | viridochromogenes |
| 3 | DEPA | 329 | T30873, 355aa | 1e−146 | 244/322 (75.78%) | 279/322 (86.65%) | dNDP-glucose dehydratase, Streptomyces |
| | | | | | | | viridochromogenes |
| | | | AAG18457.1, 332aa | 1e−127 | 223/326 (68.4%) | 257/326 (78.83%) | AprE, Streptomyces tenebrarius |
| | | | AAC68681.1, 337aa | 1e−120 | 214/328 (65.24%) | 245/328 (74.7%) | TDP-glucose-4,6-dehydratase, |
| | | | | | | | Streptomyces venezuelae |
| 4 | DEPJ | 342 | AAK83179.1, 342aa | 1e−160 | 277/342 (80.99%) | 302/342 (88.3%) | putative NDP-glucose 4-epimerase, |
| | | | | | | | Streptomyces viridochromogenes |
| | | | NP_228319.1, 309aa | 1e−43 | 113/305 (37.05%) | 164/305 (53.77%) | UDP-glucose 4-epimerase, putative, |
| | | | | | | | Thermotoga maritima |
| | | | NP_252757.1, 309aa | 1e−36 | 111/303 (36.63%) | 155/303 (51.16%) | probable epimerase, |
| | | | | | | | Pseudomonas aeruginosa |
| 5 | UNAJ | 245 | AAK83180.1, 199aa | 9e−59 | 116/198 (58.59%) | 137/198 (69.19%) | putative methyltransferase, |
| | | | | | | | Streptomyces viridochromogenes |
| 6 | OXRW | 224 | AAK83181.1, 223aa | 3e−80 | 146/218 (66.97%) | 167/218 (76.61%) | putative oxygenase, Streptomyces |
| | | | | | | | viridochromogenes |
| | | | AAF01812.1, 267aa | 5e−10 | 61/231 (26.41%) | 93/231 (40.26%) | SnoK, Streptomyces nogalater |
| | | | AAK83187.1, 314aa | 2e−08 | 54/212 (25.47%) | 86/212 (40.57%) | putative oxygenase, Streptomyces |
| | | | | | | | viridochromogenes |
| 7 | GTFA | 423 | AAK83182.1, 430aa | 1e−177 | 293/418 (70.1%) | 346/418 (82.78%) | putative glycosyltransferase, Streptomyces |
| | | | | | | | viridochromogenes |
| | | | T46681, 411aa | 1e−60 | 151/417 (36.21%) | 223/417 (53.48%) | probable glycosyltransferase snogZ, imported, |
| | | | | | | | Streptomyces nogalater |
| | | | AAD55583.1, 396aa | 9e−43 | 134/414 (32.37%) | 200/414 (48.31%) | glycosyltransferase, Streptomyces |
| | | | | | | | argillaceus |
| 8 | MTFD | 247 | AAK83184.1, 240aa | 4e−67 | 125/226 (55.31%) | 157/226 (69.47%) | putative methyltransferase, |
| | | | | | | | Streptomyces viridochromogenes |
| 9 | OXRF | 342 | AAK83185.1, 330aa | 2e−77 | 163/338 (48.22%) | 193/338 (57.1%) | putative oxidoreductase, |
| | | | | | | | Streptomyces viridochromogenes |
| | | | NP_437022.1, 331aa | 9e−32 | 94/302 (31.13%) | 135/302 (44.7%) | putative aldoketo reductase protein, |
| | | | | | | | Sinorhizobium meliloti |
| | | | NP_388834.1, 331aa | 4e−30 | 84/295 (28.47%) | 137/295 (46.44%) | similar to aldo/keto reductase, |
| | | | | | | | Bacillus subtilis |
| 10 | MTFE | 257 | AAK83186.1, 239aa | 2e−87 | 157/219 (71.69%) | 172/219 (78.54%) | putative methyltransferase, |
| | | | | | | | Streptomyces viridochromogenes |
| | | | NP_419820.1, 453aa | 3e−07 | 55/164 (33.54%) | 80/164 (48.78%) | hypothetical protein, |
| | | | | | | | Caulobacter crescentus |
| 11 | OXRV | 314 | AAK83187.1, 314aa | 1e−136 | 227/303 (74.92%) | 252/303 (83.17%) | putative oxygenase, Streptomyces, |
| | | | | | | | viridochromogenes |
| | | | AAK83181.1, 223aa | 9e−10 | 65/217 (29.95%) | 96/217 (44.24%) | putative oxygenase, Streptomyces |
| | | | | | | | viridochromogenes |
| 12 | MTFF | 282 | AAK83188.1, 277aa | 1e−103 | 179/241 (74.27%) | 199/241 (82.57%) | AviG3, Streptomyces |
| | | | | | | | viridochromogenes |
| | | | NP_489410.1, 209aa | 4e−07 | 40/136 (29.41%) | 63/136 (46.32%) | probable methyltransferase, |
| | | | | | | | Nostoc PCC 7120 |
| 13 | MTFV | 426 | AAK83176.1, 407aa | 1e−75 | 183/423 (43.26%) | 233/423 (55.08%) | methyltransferase, Streptomyces |
| | | | | | | | viridochromogenes |
| | | | AAD41823.1, 418aa | 2e−74 | 171/412 (41.5%) | 225/412 (54.61%) | NDP-hexose 3-C-methyltransferase, |
| | | | | | | | Streptomyces fradiae |
| 14 | UEVA | 397 | AAK83189.1, 396aa | 0.0 | 311/394 (78.93%) | 336/394 (85.28%) | AviX12, Streptomyces viridochromogenes |
| 15 | MEMK | 445 | CAC48373.1, 452aa | 3e−74 | 159/437 (36.38%) | 212/437 (48.51%) | putative antiporter, Amycolatopsis |
| | | | | | | | mediterranei |
| | | | NP_103653.1, 737aa | 2e−31 | 94/395 (23.8%) | 153/395 (38.73%) | Na/H antiporter, Mesorhizobium loti |
| | | | AAF26906.1, 713aa | 5e−22 | 90/376 (23.94%) | 135/376 (35.9%) | unknown, Polyangium cellulosum |
| 16 | PKSO | 1266 | T30871, 1293aa | 0.0 | 826/1267 (65.19%) | 951/1267 (75.06%) | orsellinic acid synthase, Streptomyces |
| | | | | | | | viridochromogenes |
| | | | AAK48943.1, 1778aa | 0.0 | 451/1171 (38.51%) | 632/1171 (53.97%) | 6-methylsalicylic acid synthase, |
| | | | | | | | Byssochlamys nivea |
| | | | BAA20102.1, 1800aa | 0.0 | 466/1175 (39.66%) | 652/1175 (55.49%) | 6-methylsalicylic acid synthase, |
| | | | | | | | Aspergillus terreus |
| 17 | KASA | 344 | AAK83178.1, 343aa | 1e−128 | 229/343 (66.76%) | 265/343 (77.26%) | AviN, Streptomyces viridochromogenes |
| | | | AAG29787.1, 355aa | 5e−72 | 144/347 (41.5%) | 203/347 (58.5%) | 3-ketoacyl-ACP-synthase, Streptomyces |
| | | | | | | | rishiriensis |
| | | | CAB71914.1, 346aa | 5e−69 | 134/343 (39.07%) | 199/343 (58.02%) | hypothetical protein, Streptomyces |
| | | | | | | | coelicolor |
| 18 | MTFG | 353 | AAL02176.1, 341aa | 2e−11 | 78/345 (22.61%) | 128/345 (37.1%) | rRNA methyltransferase, Enterococcus |
| | | | | | | | faecium |
| | | | NP_147325.1, 354aa | 2e−09 | 92/365 (25.21%) | 132/365 (36.16%) | hypothetical protein, Aeropyrum pernix |
| 19 | OXCO | 493 | AAK61713.1, 475aa | 1e−102 | 201/458 (43.89%) | 268/458 (58.52%) | oxidoreductase-like protein, |
| | | | | | | | Streptomyces aureofaciens |
| | | | AAL82808.1, 431aa | 1e−88 | 183/382 (47.91%) | 230/382 (60.21%) | JadZ, Streptomyces venezuelae |
| | | | CAB75422.1, 602aa | 2e−61 | 169/487 (34.7%) | 236/487 (48.46%) | polyphenol oxidase, Acremonium murorum |
| 20 | HOXM | 492 | AAK81830.1, 497aa | 3e−59 | 139/390 (35.64%) | 211/390 (54.1%) | non-heme halogenase, Streptomyces |
| | | | | | | | lavendulae |
| | | | T30590, 491aa | 5e−59 | 134/374 (35.83%) | 201/374 (53.74%) | alkylhalidase homolog, Amycolatopsis |
| | | | | | | | orientalis |
| | | | CAA76550.1, 491aa | 2e−58 | 133/374 (35.56%) | 201/374 (53.74%) | halogenase, Amycolatopsis mediterranei |
| 21 | EFFA | 530 | AAF00219.1, 525aa | 1e−122 | 237/498 (47.59%) | 310/498 (62.25%) | transporter, Streptomyces fradiae |
| | | | T36377, 531aa | 1e−111 | 209/488 (42.83%) | 290/488 (59.43%) | probable exporter, Streptomyces |
| | | | | | | | coelicolor |
| | | | AAD13557.1, 517aa | 1e−108 | 206/491 (41.96%) | 284/491 (57.84%) | LanJ, Streptomyces cyanogenus |
| 22 | DEPE | 346 | CAC20923.1, 343aa | 1e−122 | 210/326 (64.42%) | 256/326 (78.53%) | PimJ protein, Streptomyces natalensis |
| | | | AAK73500.1, 344aa | 1e−121 | 210/326 (64.42%) | 262/326 (80.37%) | AmphDIII, Streptomyces nodosus |
| | | | AAF71765.1, 344aa | 1e−120 | 212/326 (65.03%) | 255/326 (78.22%) | NysD III, Streptomyces noursei |
| 23 | DHYA | 484 | CAB96551.1, 486aa | 1e−119 | 220/444 (49.55%) | 276/444 (62.16%) | D-olivose, D-oliose and D-mycarose 2,3- |
| | | | | | | | dehydratase, Streptomyces argillaceus |
| | | | T51103, 474aa | 1e−118 | 230/446 (51.57%) | 278/446 (62.33%) | 2,3-dehydratase, validated, |
| | | | | | | | Streptomyces antibioticus |
| | | | AAG23276.1, 486aa | 1e−117 | 223/457 (48.8%) | 281/457 (61.49%) | probable NDP-hexose-2,3- |
| | | | | | | | dehydratase, Saccharopolysporainosa |
| 24 | OXRA | 344 | AAG23275.1, 332aa | 2e−71 | 141/298 (47.32%) | 174/298 (58.39%) | probable NDP-hexose-3- |
| | | | | | | | ketoreductase, Saccharopolysporainosa |
| | | | AAC01734.1, 330aa | 2e−68 | 140/318 (44.03%) | 180/318 (56.6%) | oxidoreductase, Amycolatopsis |
| | | | | | | | mediterranei |
| | | | AAD13550.1, 321aa | 1e−67 | 138/317 (43.53%) | 177/317 (55.84%) | oxidoreductase homolog, Streptomyces |
| | | | | | | | cyanogenus |
| 25 | DEPG | 317 | T46669, 328aa | 1e−40 | 114/295 (38.64%) | 150/295 (50.85%) | dTDP-4-keto-6-deoxyhexose reductase, |
| | | | | | | | Streptomyces nogalater |
| | | | AAD13561.1, 326aa | 2e−40 | 106/262 (40.46%) | 136/262 (51.91%) | NDP-hexose 4-keto reductase, |
| | | | | | | | Streptomyces cyanogenus |
| | | | AAF72549.1, 346aa | 2e−38 | 120/307 (39.09%) | 150/307 (48.86%) | NDP-hexose 4-ketoreductase, |
| | | | | | | | Streptomyces fradiae |
| 26 | REBV | 82 | AAK83173.1, 192aa | 2e−22 | 50/74 (67.57%) | 62/74 (83.78%) | putative response regulator, |
| | | | | | | | Streptomyces viridochromogenes |
| | | | AAF71781.1, 210aa | 5e−21 | 46/73 (63.01%) | 61/73 (83.56%) | ORF4, Streptomyces noursei |
| | | | BAB69313.1, 232aa | 2e−18 | 43/73 (58.9%) | 56/73 (76.71%) | transcription regulatory protein, |
| | | | | | | | Streptomyces avermitilis |
| 27 | GTFG | 304 | AAK83170.1, 240aa | 1e−61 | 119/193 (61.66%) | 135/193 (69.95%) | putative glycosyltransferase, |
| | | | | | | | Streptomyces viridochromogenes |
| | | | NP_487216.1, 313aa | 2e−04 | 25/69 (36.23%) | 32/69 (46.38%) | probable glycosyl transferase, |
| | | | | | | | Nostoc. PCC 7120 |
| 28 | DEPI | 423 | AAD45554.1, 312aa | 2e−52 | 117/283 (41.34%) | 151/283 (53.36%) | Spcl, Streptomyces netropsis |
| | | | NP_228319.1, 309aa | 2e−26 | 90/301 (29.9%) | 139/301 (46.18%) | UDP-glucose 4-epimerase, putative, |
| | | | | | | | Thermotoga maritima |
| | | | NP_218151.1, 314aa | 7e−22 | 94/310 (30.32%) | 126/310 (40.65%) | rmlB2, Mycobacterium |
| | | | | | | | tuberculosis H37Rv |
| 29 | PHOD | 250 | | | | | No homolog by blastp in GenBank nr |
| | | | | | | | protein database |
| 30 | OXRW | 253 | AAK83181.1, 223aa | 3e−13 | 72/236 (30.51%) | 103/236 (43.64%) | putative oxygenase, Streptomyces |
| | | | | | | | viridochromogenes |
| | | | AAF01812.1, 267aa | 8e−12 | 65/238 (27.31%) | 95/238 (39.92%) | SnoK, Streptomyces nogalater |
| | | | NP_107761.1, 273aa | 9e−09 | 48/161 (29.81%) | 74/161 (45.96%) | unknown protein, Mesorhizobium loti |
| 31 | GTFH | 340 | AAK83193.1, 342aa | 1e−119 | 209/342 (61.11%) | 244/342 (71.35%) | putative glycosyltransferase, Streptomyces |
| | | | | | | | viridochromogenes |
| | | | NP_489347.1, 367aa | 5e−08 | 78/349 (22.35%) | 135/349 (38.68%) | probable glycosyl transferase, |
| | | | | | | | Nostoc. PCC 7120 |
| | | | NP_102448.1, 353aa | 3e−07 | 81/336 (24.11%) | 129/336 (38.39%) | probable sugar transferase, |
| | | | | | | | Mesorhizobium loti |
| 32 | UNKU | 351 | | | | | No homolog by blastp in GenBank nr |
| | | | | | | | protein database |
| 33 | GTFE | 347 | AAK83192.1, 338aa | 1e−129 | 222/309 (71.84%) | 247/309 (79.94%) | putative glycosyltransferase, |
| | | | | | | | Streptomyces viridochromcgenes |
| | | | AAF04375.1, 296aa | 3e−18 | 78/260 (30%) | 105/260 (40.38%) | dTDP-rhamnosyl transferase, |
| | | | | | | | Mycobacterium smegmatis |
| | | | NP_301582.1, 308aa | 8e−18 | 79/272 (29.04%) | 113/272 (41.54%) | putative dTDP-rhamnosyl transferase, |
| | | | | | | | Mycobacterium leprae |
| 34 | DEPH | 308 | AAK83169.1, 322aa | 1e−88 | 166/305 (54.43%) | 198/305 (64.92%) | putative UDP-glucose 4-epimerase, |
| | | | | | | | Streptomyces viridochromogenes |
| | | | NP_218151.1, 314aa | 4e−61 | 141/305 (46.23%) | 173/305 (56.72%) | rmlB2, Mycobacterium |
| | | | | | | | tuberculosis H37Rv |
| | | | NP_228319.1, 309aa | 3e−61 | 128/293 (43.69%) | 173/293 (59.04%) | UDP-glucose 4-epimerase, Nputative, |
| | | | | | | | Thermotoga maritima |
| 35 | GTFA | 389 | AAD13553.1, 373aa | 5e−76 | 161/377 (42.71%) | 218/377 (57.82%) | glycosyl transferase homolog, |
| | | | | | | | Streptomyces cyanogenus |
| | | | AAF00209.1, 365aa | 1e−69 | 159/371 (42.86%) | 203/371 (54.72%) | glycosyl transferase, |
| | | | | | | | Streptomyces fradiae |
| | | | T46519, 382aa | 4e−59 | 140/382 (36.65%) | 201/382 (52.62%) | probable glycosyl transferase, |
| | | | | | | | imported, Streptomyces |
| | | | | | | | violaceoruber |
| 36 | OXBN | 412 | S39965, 406aa | 2e−97 | 191/386 (49.48%) | 242/386 (62.69%) | hypothetical protein, Streptomyces |
| | | | | | | | griseus |
| | | | AAB63045.1, 342aa | 4e−53 | 114/211 (54.03%) | 136/211 (64.45%) | putative flavoprotein, Streptomyces |
| | | | | | | | peucetius |
| | | | NP_388333.1, 381aa | 5e−25 | 103/371 (27.76%) | 159/371 (42.86%) | similar to butyryl-CoA dehydrogenase, |
| | | | | | | | Bacillus subtilis |
| 37 | DATC | 373 | BAB72037.1, 369aa | 1e−147 | 262/373 (70.24%) | 294/373 (78.82%) | AclZ, Streptomyces galilaeus |
| | | | B43306, 370aa | 1e−146 | 260/369 (70.46%) | 291/369 (78.86%) | probable hydro-lyase dnrJ, |
| | | | | | | | Streptomyces peucetius |
| | | | AAG13910.1, 374aa | 1e−145 | 258/374 (68.98%) | 295/374 (78.88%) | TDP-3-keto-6-deoxyhexose 3- |
| | | | | | | | aminotransaminase, Micromonospora |
| | | | | | | | megalomicea |
| 38 | MTFV | 416 | T30587, 408aa | 1e−160 | 273/402 (67.91%) | 318/402 (79.1%) | hypothetical protein, Amycolatopsis |
| | | | | | | | orientalis |
| | | | CAC48364.1, 408aa | 1e−158 | 271/402 (67.41%) | 320/402 (79.6%) | putative C-3 methyl transferase, Amycolatopsis |
| | | | | | | | mediterranei |
| | | | AAC38444.1, 403aa | 1e−154 | 264/398 (66.33%) | 309/398 (77.64%) | daunorubicin/doxorubicin biosynthesis |
| | | | | | | | enzyme, Streptomyces |
| | | | | | | | peucetius |
| 39 | EPIM | 200 | S39966, 212aa | 9e−55 | 106/196 (54.08%) | 128/196 (65.31%) | hypothetical protein, Streptomyces |
| | | | | | | | griseus |
| | | | AAB63046.1, 208aa | 2e−54 | 103/196 (52.55%) | 130/196 (66.33%) | putative epimerase, Streptomyces |
| | | | | | | | peucetius |
| | | | CAC48377.1, 205aa | 9e−52 | 98/195 (50.26%) | 128/195 (65.64%) | putative 3, 5 epimerase, Amycolatopsis |
| | | | | | | | mediterranei |
| 40 | DEPF | 359 | T17473, 325aa | 5e−49 | 111/276 (40.22%) | 142/276 (51.45%) | hypothetical protein, Amycolatopsis |
| | | | | | | | orientalis |
| | | | AAL14256.1, 325aa | 4e−45 | 106/240 (44.17%) | 123/240 (51.25%) | NDP-4-keto-6-deoxyhexose 4-ketoreductase, |
| | | | | | | | Streptomyces venezuelae |
| | | | AAG13913.1, 328aa | 5e−44 | 117/288 (40.63%) | 141/288 (48.96%) | TDP-4-keto-6-deoxyhexose 4-ketoreductase, |
| | | | | | | | Micromonospora megalomicea subsp. nigra |
| 41 | UNBB | 309 | | | | | No homolog by blastp in GenBank nr |
| | | | | | | | protein database |
| 42 | OXRF | 354 | CAC18692.2, 345aa | 3e−57 | 136/331 (41.09%) | 185/331 (55.89%) | putative aldoketoreductase, |
| | | | | | | | Streptomyces coelicolor |
| | | | NP_108492.1, 347aa | 2e−55 | 130/335 (38.81%) | 182/335 (54.33%) | oxido-reductase, and dehydratase |
| | | | | | | | mocA, Mesorhizobium loti |
| | | | NP_386945.1, 344aa | 3e−54 | 128/311 (41.16%) | 179/311 (57.56%) | Putative oxidoreductase, |
| | | | | | | | Sinorhizobium meliloti |
| 43 | HOXG | 284 | | | | | No homolog by blastp in GenBank nr |
| | | | | | | | protein database |
| 44 | MTFA | 362 | P42712, 376aa | 2e−58 | 133/350 (38%) | 181/350 (51.71%) | O-demethylpuromycin-O-methyltransferase, |
| | | | | | | | Streptomyces alboniger |
| | | | BAA32132.1, 305aa | 4e−48 | 113/303 (37.29%) | 145/303 (47.85%) | Orf4, Streptomyces griseus |
| | | | CAB76315.1, 342aa | 1e−31 | 99/334 (29.64%) | 148/334 (44.31%) | putative O-methyltransferase, |
| | | | | | | | Streptomyces coelicolor A3(2) |
| 45 | MTLA | 251 | AAG32067.1, 250aa | 1e−48 | 104/209 (49.76%) | 125/209 (59.81%) | rRNA methyl transferase, |
| | | | | | | | Streptomyces viridochromogenes |

TABLE XXX-B

| EVEA | ORF | START (bp) . . . END (bp) | LENGTH (aa) | ORIENTATION | INTEGRITY |
|---|---|---|---|---|---|
| Contig 1 | 1 | 1 . . . 747 | 248 | POSITIVE | COMPLETE |
| | 2 | 895 . . . 1962 | 355 | POSITIVE | COMPLETE |
| | 3 | 1962 . . . 2951 | 329 | POSITIVE | COMPLETE |
| | 4 | 3054 . . . 4082 | 342 | POSITIVE | COMPLETE |
| | 5 | 4137 . . . 4874 | 245 | POSITIVE | COMPLETE |
| | 6 | 4871 . . . 5545 | 224 | POSITIVE | COMPLETE |
| | 7 | 5598 . . . 6869 | 423 | POSITIVE | COMPLETE |
| | 8 | 6946 . . . 7689 | 247 | POSITIVE | COMPLETE |
| | 9 | 7735 . . . 8763 | 342 | POSITIVE | COMPLETE |
| | 10 | 8753 . . . 9526 | 257 | POSITIVE | COMPLETE |
| | 11 | 9523 . . . 10467 | 314 | POSITIVE | COMPLETE |
| | 12 | 10464 . . . 11312 | 282 | POSITIVE | COMPLETE |
| | 13 | 11314 . . . 12594 | 426 | POSITIVE | COMPLETE |
| | 14 | 12627 . . . 13820 | 397 | POSITIVE | COMPLETE |
| | 15 | 13867 . . . 15204 | 445 | POSITIVE | COMPLETE |
| Contig 2 | 16 | 3806 . . . 6 | 1266 | NEGATIVE | COMPLETE |
| | 17 | 4893 . . . 3859 | 344 | NEGATIVE | COMPLETE |
| | 18 | 5247 . . . 6305 | 353 | POSITIVE | COMPLETE |
| | 19 | 7794 . . . 6313 | 493 | NEGATIVE | COMPLETE |
| | 20 | 9421 . . . 7943 | 492 | NEGATIVE | COMPLETE |
| | 21 | 11171 . . . 9579 | 530 | NEGATIVE | COMPLETE |
| | 22 | 11575 . . . 12615 | 346 | POSITIVE | COMPLETE |
| | 23 | 12612 . . . 14066 | 484 | POSITIVE | COMPLETE |
| | 24 | 14071 . . . 15105 | 344 | POSITIVE | COMPLETE |
| | 25 | 15122 . . . 16075 | 317 | POSITIVE | COMPLETE |
| | 26 | 17064 . . . 17312 | 82 | POSITIVE | COMPLETE |
| | 27 | 17463 . . . 18377 | 304 | POSITIVE | COMPLETE |
| | 28 | 19301 . . . 18030 | 423 | NEGATIVE | COMPLETE |
| | 29 | 20061 . . . 19309 | 250 | NEGATIVE | COMPLETE |
| | 30 | 20262 . . . 21023 | 253 | POSITIVE | COMPLETE |
| | 31 | 22144 . . . 21122 | 340 | NEGATIVE | COMPLETE |
| | 32 | 23214 . . . 22159 | 351 | NEGATIVE | COMPLETE |
| | 33 | 24254 . . . 23211 | 347 | NEGATIVE | COMPLETE |
| | 34 | 25177 . . . 24251 | 308 | NEGATIVE | COMPLETE |
| | 35 | 26343 . . . 25174 | 389 | NEGATIVE | COMPLETE |
| | 36 | 26626 . . . 27864 | 412 | POSITIVE | COMPLETE |
| | 37 | 27875 . . . 28996 | 373 | POSITIVE | COMPLETE |
| | 38 | 29105 . . . 30355 | 416 | POSITIVE | COMPLETE |
| | 39 | 30363 . . . 30965 | 200 | POSITIVE | COMPLETE |
| | 40 | 32002 . . . 30923 | 359 | NEGATIVE | COMPLETE |
| | 41 | 32933 . . . 32004 | 309 | NEGATIVE | COMPLETE |
| | 42 | 33190 . . . 34254 | 354 | POSITIVE | COMPLETE |
| | 43 | 34375 . . . 35229 | 284 | POSITIVE | COMPLETE |
| | 44 | 35226 . . . 36314 | 362 | POSITIVE | COMPLETE |
| | 45 | 36361 . . . 37116 | 251 | POSITIVE | COMPLETE |

FIG. 10 is a schematic representation comparing the everninomicin biosynthetic locus from Micromonospora carbonacae var. aurantiaca (EVER) to the everninomicin biosynthetic locus from Micromonospora carbonacea var. africana (EVEA). The scale at the top of the figure is in kilobasepairs. Solid black arrows depict the relative positions of the individual ORFs in EVER and EVEA with the arrowhead indicating the orientation of each ORF; the corresponding four letter protein family designation is indicated to the right of each ORF. The empty arrows between the two loci highlight segments that contain a number of ORFs whose relative order and orientation is identical between the two loci. The orientation of the empty arrows indicates the relative order of the ORFs in each segment; the segments in the EVER locus have all arbitrarily been assigned the “left-to-right” orientation. A segment is defined as two or more adjacent ORFs whose relative order and orientation is identical in the loci being compared. The solid lines between the two loci link each segment from one locus to the corresponding segment in the other locus. The dashed lines between the two loci link individual pairs of homologous ORFs that do not form segments.

ORFs in each locus that do not have a counterpart in the other locus are indicated by an ‘X’. EVER contains ten (10) ORFs for which no counterpart is found in EVEA; these include ORFs designated as members of the protein families MTBA, MTFH, UEVB, MTIA, OXRU, OXRT, DEPD, ENGA, REGL, and KINB. EVEA contains four (4) ORFs for which no counterpart is found in EVER; these include ORFs designated as members of the protein families HYDH, OXRF, EFFA and OXRF. ORFs of the protein families MTBA, MTFH, UEVB, MTIA, OXRU, OXRT, DEPD, ENGA, REGL, KINB, HYDH, OXRF, EFFA and OXRF are not likely to be involved in the assembly of the core structure of the everninomicin-type orthosomycins. Rather, they are believed to be involved in various modifications of the core structure including methylation (MTBA and MTFH); oxidation/reduction (OXRU, OXRT, OXRF); or in resistance mechanisms (MTIA, EFFA).

A search of NCBI's Conserved Domain Database with Reverse Position Specific BLAST (Altschul et al., (1997) Nucleic Acids Res. 25:3389-3402) revealed that the UEVB family displays structural homology to the double stranded beta helix domain involved in carbohydrate binding and in protein-protein interactions in different contexts. Thus the UEVB family may represent small, carbohydrate-binding proteins that may specifically recognize certain substructures of orthosomycins. One interesting possibility is that the UEVB proteins recognize and bind to the sugar residue H so as to block further modifications. This hypothesis is based on the fact that the everninomicin locus from Micromonospora carbonacae var. africana does not contain a UEVB homologue and that this organism has been described to produce everninomicins with various substitutions on sugar residue H, including an ester linkage to an orsellinic acid moiety. Thus, based on this hypothesis, one would predict that disruption of the UEVB ORF in the AVIA, AVIL, or EVER loci or other orthosomycin loci that may contain such an ORF may result in the production of new orthosomycins with additional substitutions in sugar residue H.

The finding that the ORFs of the EVER and EVEA loci are shuffled to such an extent and the presence of ORFs that have no counterparts in each locus is unexpected as both loci produce related compounds and the respective organisms containing these loci are both classified as Micromonospora carbonacae.

It is to be understood that the embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes.

1. A method of identifying an orthosomycin biosynthetic gene or gene fragment comprising providing a sample containing genomic DNA, and detecting the presence of a nucleic acid sequence coding for a polypeptide from at least two of the groups consisting of:
a. SEQ ID NO: 51; Genbank accession no. AAK83192; SEQ ID NO: 53; SEQ ID NO: 55; and polypeptides having at least 65% homology to a polypeptide having the sequence of SEQ ID NOS: 51, 53, 55 or Genbank accession no AAK83192;
b. SEQ ID NO: 57; Genbank accession no. AAK83170; SEQ ID NO: 59; SEQ ID NO: 61; and polypeptides having at least 65% homology to a polypeptide having the sequence of SEQ ID NOS: 67, 59, 61 or Genbank accession no, AAKS3170;
c. SEQ ID NO: 63, Genbank accession no. AAK83193, SEQ ID NO: 65, SEQ ID NO: 67, and polypeptides having at least 65% homology to a polypeptide having the sequence of SEQ ID NOS: 63, 65, 67 or Genbank accession no. AAK83193;
d. SEQ ID NO: 69, SEQ ID NO: 71, SEQ ID NO: 73, and polypeptides having at least 66% homology to a polypeptide having the sequence of SEQ ID NOS: 69, 71 or 73;
e. SEQ ID NO: 99, Genbank accession no. AAK83184, SEQ ID NO: 101, SEQ ID NO: 103, and polypeptides having at least 65% homology to a polypeptide having the sequence of SEQ ID NOS: 99, 101, 103 or Genbank accession no. AAK83184;
f. SEQ ID NO: 105, Genbank accession no. AAK83186, SEQ ID NO: 107, SEQ ID NO: 109, and polypeptides having at least 65% homology to a polypeptide having the sequence of SEQ ID NOS: 105, 107, 109 or Genbank accession no. AAK83186;
g. SEQ ID NO: 111, Genbank accession no. AAK83188, SEQ ID NO: 113, SEQ ID NO: 115, and polypeptides having at least 65% homology to a polypeptide having the sequence of SEQ ID NOS: 111, 113, 115 or Genbank accession no. AAK83188;
h. SEQ ID NO: 127, Genbank accession no AAG32067, SEQ ID NO: 129, SEQ ID NO: 131 and polypeptides having at least 65% homology to a polypeptide having the sequence of SEQ ID NOS: 127, 129, 131 or Genbank accession no. AAG32067;
i. SEQ ID NO: 123, Genbank accession no. AAG32066, SEQ ID NO: 125 and polypeptides having at least 65% homology to a polypeptide having the sequence of SEQ ID NOS: 123, 125 or Genbank accession no. AAG32066;
j. SEQ ID NO: 153, Genbank accession no; AAK83187, SEQ ID NO: 155, SEQ ID NO: 157, and polypeptides having at least 65% homology to a polypeptide having the sequence of SEQ ID NOS: 153, 155, 157 or Genbank accession no. AA83187
k. SEQ ID NO: 159, SEQ ID NO: 161, SEQ ID NO: 163 and polypeptides having at least 65% homology to a polypeptide having the sequence of SEQ ID NOS: 159, 161 or 163;
l. SEQ ID NO: 167, SEQ ID NO: 173, Genbank accession no. AAK83181, SEQ ID NO: 169 and polypeptides having at least 65% homology to a polypeptide having the sequence of SEQ ID NOS: 167, 169, 173 or Genbank accession no. AAK83181;
m. SEQ ID NO: 175, SEQ ID NO: 177, SEQ ID NO: 179 and polypeptides having at least 65% homology to a polypeptide having the sequence of SEQ ID NOS: 175, 177 or 179;
n. SEQ ID NO: 165, SEQ ID NO: 171, SEQ ID NO: 169 and polypeptides having at least. 65% homology to a polypeptide having the sequence of SEQ ID NOS: 165, 169 or 171;
o. SEQ ID NO: 193, Genbank accession no. AAK83189, SEQ ID NO: 195, SEQ ID NO: 197 and polypeptides having at least 65% homology to a polypeptide having the sequence of SEQ ID NOS: 193, 195, 197 or Genbank accession no. AA3189;
p. SEQ ID NO: 199, Genbank accession no. AAK83174, SEQ ID NO: 201 and polypeptides having at least 65% homology to a polypeptide having the sequence of SEQ ID NOS: 199, 201 or Genbank accession no. AAK83174; and
q. SEQ ID NO: 203, SEQ ID NO: 205, SEQ ID NO: 207 and polypeptides having at least 65% homology to a polypeptide having the sequence of SEQ ID NOS: 203, 205 or 207.

2. The method according to claim 1 further comprising the step of detecting the presence of either:
a. a nucleic acid sequence coding for a polypeptide from at least one of the of the groups consisting of:
r. SEQ ID NO: 209, SEQ ID NO: 211 and polypeptides having at least 65% homology to a polypeptide of SEQ ID NO: 209 or SEQ ID NO. 211;
s. SEQ ID NO: 213, SEQ ID NO: 215 and polypeptides having at least 65% homology to a polypeptide of SEQ ID NO: 213 or SEQ ID NO: 215;
t. SEQ ID NO: 217, SEQ ID NO: 219 and polypeptides having at least 65% homology to a polypeptide of SEQ ID NO: 217 or SEQ ID NO: 219;
u. SEQ ID NO: 221, SEQ ID NO: 223 and polypeptides having at least 65% homology to a polypeptide of SEQ ID NO: 221 or SEQ ID NO: 223;
v. SEQ ID NO: 225, SEQ ID NO: 227 and polypeptides having at least 65% homology to a polypeptide of SEQ ID NO: 225 or SEQ ID NO: 227;
w. SEQ ID NO: 229, SEQ ID NO: 231 and polypeptides having at least 65% homology to a polypeptide of SEQ ID NO: 229 or SEQ ID NO: 231;
x. SEQ ID NO: 233, SEQ ID NO: 235 and polypeptides having at least 65% homology to a polypeptide of SEQ ID NO: 233 or SEQ ID NO: 235;
y. SEQ ID NO: 237, SEQ ID NO: 239 and polypeptides having at least 65% homology to a polypeptide of SEQ ID NO: 237 or SEQ ID NO: 239; and
z. SEQ ID NO: 241, SEQ ID NO. 243 and polypeptides having at least 65% homology to a polypeptide of SEQ ID NO: 241 or SEQ ID NO: 243;or;
b. detecting the presence of a nucleic acid sequence coding for a polypeptide from at least one of the groups consisting of:
aa. SEQ ID NO: 245, Genbank accession no. AAG32068 and polypeptides having at least 65% homology to a polypeptide of SEQ ID NO: 245 or Genbank accession no. AAG32066;
bb. SEQ ID NO: 247, Genbank accession no. AAK83183, and polypeptides having at least 65% homology to a polypeptide of SEQ ID NO; 247 or Genbank accession no. AAK83183;
cc. SEQ ID NO: 249, accession no. AAG32069, and polypeptides having at least 65% homology to a polypeptide of SEQ ID NO: 249 or Genbank accession no. AA32069;
dd. SEQ ID NO: 251, Genbank accession no. AAK83172, and polypeptides having at least 65% homology to a polypeptide of SEQ ID NO; 251 or Genbank accession no. AAK83172;
ee. SEQ ID NO: 253, Genbank accession no. AAK83171 and polypeptides having at least 65% homology to a polypeptide of SEQ ID NO: 253 or Genbank accession no. AAK83171; and
ff. SEQ ID NO: 255, Genbank accession no. AAK83175, and polypeptides having at least 66% homology to a polypeptide of SEQ ID NO: 255 or Genbank accession no. AA83175.

3. The method according to claim 1 further comprising the step of using the nucleic acid sequence detected to isolate an orthosomycin gene cluster from the sample containing genomic DNA.

4. The method of claim 1 further comprising identifying an organism containing the nucleic acid sequence detected from the genomic DNA in the sample.

5. A method according to claim 1 wherein the sample containing DNA Is biomass from environmental sources.

6. A method according to claim 5 wherein the biomass is a mixed microbial culture.

7. A method according to claim 1 wherein the sample containing genomic DNA is obtained from a mixed population of organisms.

8. A method according to claim 1 wherein the sample containing genomic DNA is a genomic library containing a plurality of clones, wherein the DNA for generating the clones is obtained from a mixed population of organisms.

9. A method according to claim 1 wherein the sample containing genomic DNA is obtained from a pure culture.

10. A method according to claim 1 wherein the sample containing genomic DNA is a genomic library containing a plurality of clones, wherein the DNA for generating the clones is obtained from a pure culture.

11. A method according to claim 1 wherein the presence of a nucleic acid sequence from at least 4 of the groups (a) to (q) is detected.

12. A method according to claim 1 wherein detecting the presence of a nucleic acid sequence coding for a polypeptide from groups (a) to (q) involves use of a hybridization probe or PCR primer derived from:
a. an isolated, purified, or enriched nucleic acid comprising one of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 or the sequences complementary thereto;
or
b. an isolated, purified or enriched nucleic acid which encodes one or the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207 or fragments comprising at least 5, 10, 15, 20, 25, 30, 35, 40, 50, 75, 100 or 150 consecutive amino adds of one of the polypeptides of SEQ ID NOS: 51, 53, 55, 57, 59, 61, 63, 65, 67, 69, 71, 73, 99, 101, 103, 105, 107, 109, 111, 113, 115, 123, 125, 127, 129, 131, 153, 155, 157, 159, 161, 163, 165, 167, 169, 171, 173, 175, 177, 179, 193, 195, 197, 199, 201, 203, 205, 207.

13. A method of identifying an everninomicin-type orthosomycin biosynthetic gene duster comprising providing a sample containing DNA, and detecting the presence of a nucleic acid sequence coding for a polypeptide from at least one of the of the groups consisting of:
r. SEQ ID NO: 209, SEQ ID NO: 211 and polypeptides having at least 65% homology to a polypeptide of SEQ ID NO: 209 or SEQ ID NO: 211;
s. SEQ ID NO: 213, SEQ ID NO: 215 and polypeptides having at least 65% homology to a polypeptide of SEQ ID NO: 213 or SEQ ID NO: 215;
t. SEQ ID NO: 217, SEQ ID NO: 219 and polypeptides having at least 65% homology to a polypeptide of SEQ ID NO: 217 or SEQ ID NO: 219;
u. SEQ ID NO: 221, SEQ ID NO: 223 and polypeptides having at least 65% homology to a polypeptide of SEQ ID NO: 221 or SEQ ID NO: 223;
v. SEQ ID NO: 225, SEQ ID NO: 227 and polypeptides having at least 65% homology to a polypeptide of SEQ ID NO: 225 or SEQ ID NO: 227
w. SEQ ID NO: 229, SEQ ID NO: 231 and polypeptides having at least 65% homology to a polypeptide of SEQ ID NO: 229 or SEQ ID NO: 231;
x. SEQ ID NO: 233, SEQ ID NO: 235 and polypeptides having at least 65% homology to a polypeptide of SEQ ID NO: 233 or SEQ ID NO: 235;
y. SEQ ID NO: 237, SEQ ID NO: 239 and polypeptides having at least 65% homology to a polypeptide of SEQ ID NO: 237 or SEQ ID NO: 239; and
z. SEQ ID NO: 241, SEQ ID NO: 243 and polypeptides having at least 65% homology to a polypeptide of SEQ ID NO: 241 or SEQ ID NO: 243.

14. The method according to claim 13 further comprising the step of detecting the presence of a nucleic acid sequence coding for a polypeptide from at least two of groups (a) to (q) recited in claim 1.

15. A method according to claim 13 wherein detecting the presence of a nucleic acid sequence coding for a polypeptide from at least two of the groups (a) to (i) involves use of a hybridization probe or PCR primer of:
a. an isolated, purified, or enriched nucleic add comprising one of the sequences of SEQ ID NOS: 210, 212, 214, 216, 218, 220, 222, 224, 226, 228, 230, 232, 234, 236, 238, 240, 242, 244, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 210, 212, 214, 216, 218, 220, 222, 224, 226, 228, 230, 232, 234, 236, 238, 240, 242, 244 or the sequences complementary thereto;
or
b. an isolated, purified or enriched nucleic acid which encodes one or the polypeptides of SEQ ID NOS: 209, 211, 213, 215, 217, 219, 221, 223, 225, 227, 229, 231, 233, 235, 237, 239, 241, 243 or fragments comprising at least 5, 10, 15, 20, 25, 30, 35, 40, 60, 75, 100 or 150 consecutive amino acids of one of the polypeptides of SEQ ID NOS: 209, 211, 213, 215, 217, 219, 221, 223, 225, 227, 229, 231, 233, 235, 237, 239, 241, 243.

16. A method according to claim 15 further comprising the step of using the detected nucleic add sequence to isolate an everninomicin-type orthosomycin biosynthetic gene cluster from the sample containing the genomic DNA.

17. An everninomicin-type orthosomycin biosynthetic gene cluster obtained from claim 16.

18. A method of identifying an avilamycin-type orthosomycin biosynthetic gene cluster comprising providing a sample containing DNA, and detecting the presence of a nucleic acid sequence coding for a polypeptide from at least one of the groups consisting of
aa. SEQ ID NO: 245, Genbank accession no AAG32068 and polypeptides having at least 65% homology to a polypeptide of SEQ ID NO: 245 or Genbank accession no. AAG32068;
bb. SEQ ID NO: 247, Genbank accession no. AAK3183, and polypeptides having at least 65% homology to a polypeptide of SEQ ID NO: 247 or Genbank accession no. AAK83183;
cc. SEQ ID NO: 249, accession no. AAG32069, and polypeptides having at least 65% homology to a polypeptide of SEQ ID NO: 249 or Genbank accession no. AA32069;
dd. SEQ ID NO: 251, Genbank accession no AAK83172, and polypeptides having at least 65% homology to a polypeptide of SEQ ID NO: 251 or Genbank accession no. AAK83172;
ee. SEQ ID NO: 253, Genbank accession no. AAK83171 and polypeptides having at least 65% homology to a polypeptide of SEQ ID NO: 253 or Genbank accession no. AAK83171; and
ff. SEQ ID NO: 255, Genbank accession no. AAK83175, and polypeptides having at least 65% homology to a polypeptide of SEQ ID NO: 255 or Genbank accession no. AAK83175.

19. The method according to claim 18 further comprising the step of detecting the presence of a nucleic acid sequence coding for a polypeptide from at least two of the groups recited in claim 1.

20. A method according to claim 18 wherein detecting the presence of a nucleic acid sequence coding for a polypeptide from at least two of the groups (aa) to (ff) involves use of a hybridization probe or PCR primer of:
aa. an isolated, purified, or enriched nucleic acid comprising one of the sequences of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; the sequences complementary thereto; or a fragment comprising at least 10, 15, 20, 25, 30, 35, 40, 50, 75, 100, 150, 200, 300, 400 or 500 consecutive bases of one of the sequences of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175; or the sequences complementary thereto; or
bb. an isolated, purified or enriched nucleic acid which encodes one or the polypeptides of SEQ ID NOS: 245, 247, 249, 251, 253 or Genbank accession nos: AA32068, AAK83183, AAG32069, AAK83172, AAK83171 and AAK83175 or fragments comprising at least 5, 10, 15, 20, 25, 30, 35, 40, 50, 75, 100 or 150 consecutive amino acids of one of the polypeptides of SEQ ID NOS: 245, 247, 249, 251, 253 or Genbank accession nos: AAG32068, AAK83183, AAG32069, AAK83172, AAK83171 and AAK83175.

21. A method according to claim 18 further comprising the step of using the detected nucleic acid sequences to isolate an avilamycin-type orthosomycin biosynthetic gene cluster from the sample containing the genomic DNA

22. An evilamycin-type orthosomycin biosynthetic gene cluster obtained from claim 21.

23. A computer readable medium having stored thereon a sequence selected from the group consisting of:
a. SEQ ID NOS: 52, 54, 56, 68, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208;
b. fragments of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208 comprising at least 10 consecutive nucleotides of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208;
c. nucleodde sequences at least 70% identical to SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208, or at least 70% identical to fragments of SEQ ID NOS: 52, 54, 56, 58, 60, 62, 64, 66, 68, 70, 72, 74, 100, 102, 104, 106, 108, 110, 112, 114, 116, 124, 126, 128, 130, 132, 154, 156, 158, 160, 162, 164, 166, 168, 170, 172, 174, 176, 178, 180, 194, 196, 198, 200, 202, 204, 206, 208; and
d. sequences complementary to the sequences of (a) (b) and (c);
for use In the detection of orthosomycin-genes, orthosomycin gene fragments, orthosomycin gene clusters and orthosomycin producing organisms.

24. A computer readable medium having stored thereon a sequence selected from the group consisting of:
a SEQ ID NOS: 210, 212, 214, 216, 218, 220, 222, 224, 226, 228, 230, 232, 234, 236, 238, 240, 242, 244;
b. fragments of SEQ ID NOS: 210, 212, 214, 216, 218, 220, 222, 224, 226, 228, 230, 232, 234, 236, 238, 240, 242, 244 comprising at least 10 consecutive nucleotides of SEQ ID NOS: 210, 212, 214, 216, 218, 220, 222, 224, 226, 228, 230, 232, 234, 236, 238, 240, 242, 244;
c. nucleotide sequences having at least 70% identical to SEQ ID NOS: 210, 212, 214, 216, 218, 220, 222, 224, 226, 228, 230, 232, 234, 236, 238, 240, 242, 244, or 70% identical to fragments of SEQ ID NOS: 210, 212, 214, 216, 218, 220, 222, 224, 226, 228, 230, 232, 234, 236, 238, 240, 242, 244; and
d. sequences complementary to the sequences of (a) (b) and (c);
for use in identifying everninomycin-type orthosomycin genes, gene fragments, gene clusters or everninomycin-type orthosomycin-producing organisms.

25. A computer readable medium having stored thereon a sequence selected from the group consisting of:
a. SEQ ID NOS: 246, 248, 250, 252, 254, 256 and the nucleic acid sequences corresponding to Genbank accession nos. AAG32068, AAK83183, AAG32069, AAK83172, AAK83171 and MK83175;
b. fragments of SEQ ID NOS: 246, 248, 250, 252, 254, 256 and the nucleic acid sequences corresponding to Genbank accession nos. AAG32068, AAK83183, AAG32069, AAK83172, AAK83171 and AAK83175 comprising at least 10 consecutive nucleotides of SEQ ID NOS: 246, 248, 250, 252, 254, 256 and the nucleic acid sequences corresponding to Genbank accession nos. AAG32068, AAK3183, AAG32069, AAK83172, AAK83171 and AAK83175;
c. nucleotide sequences 70% identical to SEQ ID NOS: 246, 248, 250, 252, 254, 256 or the nucleic acid sequences corresponding to Genbank accession nos. AAG32068, AAK83183, AAG32069, AAK83172, AAK83171 and AAK83175; or 70% identical to fragments of SEQ ID NOS: 246, 248, 250, 252, 254, 256 or the nucleic acid sequences corresponding to Genbank accession nos AAG32068, AAK83183, AAG32069, AAK83172, AAK3171 and AAK83175; and
d. sequences complementary to the sequences of (a) (b) and (c);
for use in identifying avilamycin-type orthosomycin genes, gene fragments, gene clusters or avilamycin-type orthosomycin producing organisms.
